(19) United States
(12) Patent Application Publication
(10) Pub. No.: US 2017/0037007 A1
(43) Pub. Date: Feb. 9, 2017

(54) SUBSTITUTED N-(2-(AMINO)-2OXOETHYL)BENZAMIDE INHIBITORS OF AUTOTAXIN AND THEIR PREPARATION AND USE IN THE TREATMENT OF LPA-DEPENDENT OR LPA-MEDIATED DISEASES

(71) Applicant: X-Rx, Inc., New York, NY (US)

(72) Inventors: Frank Ruebsam, Waltham, MA (US); Ce Wang, Waltham, MA (US); Haihong Ni, Waltham, MA (US); Mark J. Mulvihill, Waltham, MA (US); Lee Babiss, Waltham, MA (US); Louis Renzetti, Waltham, MA (US); Ying Zhang, Waltham, MA (US)

(21) Appl. No.: 15/303,848

(22) PCT Filed: Apr. 21, 2015
(86) PCT No.: PCT/US15/26811

§ 371 Date: Oct. 13, 2016

Related U.S. Application Data
(60) Provisional application No. 61/983,146, filed on Apr. 23, 2014.

(51) Int. Cl. C07D 211/58; C07D 211/66; A61K 45/06; C07D 401/12; A61K 31/454; A61K 31/4468; A61K 31/45

(52) U.S. Cl. CPC C07D 211/58; A61K 31/4468; C07D 211/66; A61K 31/45; C07D 401/12; A61K 31/454; A61K 45/06

(57) ABSTRACT

The present invention relates to compounds according to Formula I and pharmaceutically acceptable salts, synthesis, intermediates, formulations, and methods of disease treatment therewith, including cancer, lymphocyte homing, chronic inflammation, neuropathic pain, fibrotic diseases, thrombosis, and cholestatic pruritus, mediated at least in part by ATX.

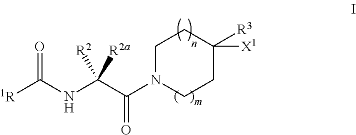

### CROSS-REFERENCE TO RELATED APPLICATIONS

This application claims the benefit under 35 U.S.C. 119(e) of U.S. Provisional Patent Application No. 61/983,146, filed on Apr. 23, 2014 and entitled “Substituted N-(2-(Amino)-2-Oxoethyl)Benzamide Inhibitors Of Autotaxin And Their Preparation And Use In The Treatment Of LPA-Dependent Or LPA-Mediated Diseases.” The entire contents of the above-referenced application is incorporated herein by reference.

### FIELD AND BACKGROUND

Autotaxin (ATX) is a secreted enzyme of the ectonucleotide pyrophosphatase/phosphodiesterase family, and is also known as Ectonucleotide Pyrophosphatase/Phosphodiesterase 2 (ENPP-2 or NPP2). ATX plays a role in driving pathological conditions, including fibrosis, arthritic inflammation, neurodegeneration, neuropathic pain, and cancer. ATX is the fundamental regulator of the conversion of Lysophosphatidylcholine (LPC) to Lysophosphatidic Acid (LPA). LPA a bioactive lipid with that affects migration, proliferation, and survival of various cell types.

Inhibition of ATX has been shown to reduce LPA levels in pathological settings. Reduction of LPA may provide therapeutic benefits in diseases with unmet medical need, including cancer, lymphocyte homing, chronic inflammation, neuropathic pain, fibrotic diseases such as Idiopathic Pulmonary Fibrosis (IPF), thrombosis, and cholestatic pruritus which are caused, mediated and/or propagated by increased LPA levels and/or activation of ATX.

Fibrotic diseases are chronic, debilitating and often lethal pathologies driven by a dysregulated response to tissue or organ injury. Fibrosis can develop in the liver, kidney, lung, dermis, vasculature, gut and other sites. Fibrosis develops due to action of pathways including growth factors, cytokines, integrin and lipids.

ATX, LPA, and LPA Receptor (LPAR) pathways have been implicated in fibrotic disease. For example, profiling studies show increased levels of ATX, LPA and LPARs in various rodent models of fibrosis and in human patient fluids and biopsy tissue. LPA can induce proliferative, survival, and chemotactic responses in transformed cell lines, indicating that LPA may exert pro-inflammatory and pro-fibrotic responses in cells known to be critical in fibrotic disease, including: fibroblasts, smooth muscle cells, macrophages, epithelial and endothelial cells, and leukocytes. Gene-targeted mouse models have implicated LPARs in fibrosis pathogenesis. Inhibitors of LPARs indicate that antagonism of receptors within this pathway blocked or reversed fibrosis in the lung, liver, kidney and skin in rodents. Cell type-specific gene targeting studies have showed that ATX plays a role in the development of lung fibrosis and inflammatory arthritis.

ATX and LPA have also been implicated in tumor progression and metastasis. ATX may be responsible for increased LPA levels in ascites and plasma of ovarian cancer patients since ATX converts LPC to LPA. Increased levels of LPA, altered receptor expression and altered responses to LPA may contribute to initiation, progression or outcome of ovarian cancer. LPA has also been linked to prostate, breast, melanoma, head and neck, bowel, brain and thyroid cancers.

LPA has been shown to promote tumor cell survival, proliferation, invasion and migration into neighboring tissues, which can result in the formation of metastases. Additionally, LPA promotes cytoskeletal remodeling that may enhance migratory and invasive properties of cells, which may contribute to cancer metastasis. These biological and pathobiological processes of LPA are initiated through the activation of G-protein coupled receptors.

Transcriptome analyses of more than 350 normal tissues and more than 1700 malignant tissues demonstrate that ATX is expressed in a variety of carcinomas and sarcomas, underscoring the potential contribution of LPA to metastatic disease.

Accordingly, when treating patients with diseases, such as cancer, lymphocyte homing, chronic inflammation, neuropathic pain, fibrotic diseases, thrombosis, and cholestatic pruritus it is desirable to lower LPA levels. This can be accomplished through inhibition of enzymes involved in LPA biosynthesis, such as ATX.

Since ATX is expressed in tumors and affects tumor cell proliferation and invasion into neighboring tissues both of which can lead to the formation of metastases, ATX is a target for anti-tumor therapy. Moreover, in angiogenesis, ATX, taken with other anti-angiogenetic factors, brings about blood vessel formation. Angiogenesis supplies tumors with nutrients during tumor growth. Therefore, inhibition of angiogenesis is a target for anti-tumor therapy, leading to starvation of a tumor.

ATX has also been implicated in nerve injury-induced neuropathic pain. LPA biosynthesis through ATX is the source of LPA for LPA1 receptor-mediated neuropathic pain. Therefore, targeted inhibition of ATX-mediated LPA biosynthesis may represent a novel treatment to prevent nerve injury-induced neuropathic pain.

Various publications refer to compounds that are capable of inhibiting ATX, including: WO2013061297, WO2012166415, US20120100592, WO2012024620, WO2011116867, WO2011017350, WO2011006569, WO2010115491, WO2010115491, WO2010112124, WO2010112116, WO2010063352, US20100016258, and WO2009151644.

Accordingly, there remains a need for ATX inhibitors having the potential to reach the clinic and obtain regulatory approval for use in the treatment and/or prophylaxis of physiological and/or pathophysiological conditions, such as cancer, lymphocyte homing, chronic inflammation, neuropathic pain, fibrotic diseases, thrombosis, and cholestatic pruritus which are caused, mediated and/or propagated by increased LPA levels and/or the activation of ATX.

### SUMMARY

The present invention includes certain substituted compounds described herein, their salts, preparations thereof, pharmaceutical compositions and formulations thereof, and methods of treating disease such as cancers therewith.

The present invention includes compounds of Formula I and pharmaceutically acceptable salts thereof:

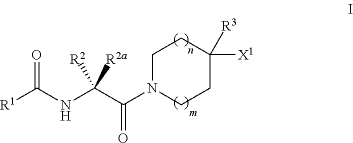

wherein X¹ is selected from —C₁₋₂alkylR⁴, —(C₀₋₂alkyl)C(O)R⁴, —(C₀₋₂alkyl)SO₂R⁴, —(C₀₋₂alkyl)NR⁴R^{4a}, —(C₀₋₂alkyl)OR⁴, or —(C₀₋₂alkyl)CR⁴R¹⁰R¹¹; m and n are each independently selected from 0, 1 or 2. Any of the above can be further substituted. Compounds of Formula I inhibit ATX.

In some embodiments, compounds of the present invention are inhibitors of ATX. In some embodiments, compounds of the present invention are selective inhibitors of ATX.

In some embodiments, the present invention includes methods of treating cancer, lymphocyte homing and chronic inflammation, neuropathic pain, fibrotic diseases, thrombosis, and cholestatic pruritus which are caused, mediated and/or propagated at least in part by increased LPA levels and/or the activation of ATX, alone or in combination regimens with other therapies.

Embodiments of the present invention include the compounds herein, pharmaceutically acceptable salts thereof, any physical forms thereof including solvates and hydrates, preparation of the compounds, intermediates, and pharmaceutical compositions and formulations thereof.

### DETAILED DESCRIPTION

In some embodiments, the present invention concerns compounds and salts thereof of Formula I, as shown below and defined herein:

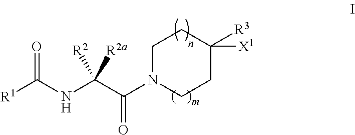

wherein:

X¹ is selected from —C₁₋₂alkylR⁴, —(C₀₋₂alkyl)C(O)R⁴, —(C₀₋₂alkyl)SO₂R⁴, —(C₀₋₂alkyl)NR⁴R^{4a}, —(C₀₋₂alkyl)OR⁴, or —(C₀₋₂alkyl)CR⁴R¹⁰R¹¹;

m and n are each independently selected from 0, 1 or 2;

R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents;

R² is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G² substituents;

R^{2a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G^{2a} substituents;

R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;

R³ is selected from —CN, C(O)NR⁷R⁸, S(O)ₙ₀R⁷R⁸, C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents;

R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents;

R^{4a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G^{4a} substituents;

G¹, G², G^{2a}, G³, G⁴, and G^{4a} are each independently selected from one or more of D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —C(O)NR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)—C(O)NR¹²R¹³, —C(O)OR¹², —C(O)—C(O)OR¹², —OC(O)R¹², —NR¹²C(O)R¹³, —NR¹²C(O)OR¹³, —NR¹²S(O)₂R¹³, —(CR¹⁴R¹⁵)C(O)R¹³, —(CR¹⁴R¹⁵)C(O)OR¹², —(CR¹⁴R¹⁵)C(O)NR¹²R¹³, —(CR¹⁴R¹⁵)ₙ₁S(O)₂NR¹²R¹³, —(CR¹⁴R¹⁵)ₙ₁NR¹²R¹³, —(CR¹⁴R¹⁵)ₙ₁OR¹², —(CR¹⁴R¹⁵)ₙ₁S(O)ₙ₂R¹², —NR¹⁶C(O)NR¹²R¹³, —NR¹⁶S(O)₂NR¹²R¹³ or —NR¹⁶S(O)NR¹²R¹³, or two G¹ substituents combine with the atoms to which they are attached to form a C₃₋₁₂cycloalkyl, C₃₋₁₂heterocycloalkyl, aryl, or heteroaryl, any of which is optionally substituted with one or more independent Q¹ substituents;

Q¹ is selected from H, D, halo, —CN, —CD₃, —OCD₃, -oxo-, —CF₃, —OCF₃, —OCHF₂, —NO₂, —B(OH)₂, —PO(OR¹⁷)₂, —PO(OR¹⁷)R¹⁸, NR¹⁷R¹⁸, —C(O)NR¹⁷OH, C₀₋₁₂alkyl-, —C₂₋₁₂alkenyl, —C₂₋₁₂alkynyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —C(O)—C(O)NR¹⁷R¹⁸, —C(O)—C(O)OR¹⁷, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁷R¹⁸, —(CR¹⁹R²⁰)ₙ₃OR¹⁷, —(CR¹⁹R²⁰)ₙ₃S(O)ₙ₄R¹⁷, —NR²¹C(O)NR¹⁷R¹⁸, —NR²¹S(O)₂NR¹⁷R¹⁸ or —NR²¹S(O)NR¹⁷R¹⁸, any of which is optionally substituted with one or more independent Q² substituents;

Q² is selected from one or more of H, D, halo, —CN, -oxo-, —CD₃, —OCD₃, —CF₃, —OCF₃, —OCHF₂, —NO₂, —B(OH)₂, —PO(OR²⁷)₂, —PO(OR²⁷)R²⁸, NR²⁷R²⁸, —C(O)NR²⁷OH, —C₂₋₁₂alkenyl, —C₂₋₁₂alkynyl, —OC₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —C(O)—C(O)NR²⁷R²⁸, —C₀₋₁₂alkylC(O)OR²⁷, —C(O)—C(O)OR²⁷, —OC(O)R²⁷, —NR²⁷C(O)R²⁸, —NR²⁷C(O)OR²⁸, —NR²⁷S(O)₂R²⁸, (CR²⁹R³⁰)ₙ₅C(O)R²⁷, —(CR²⁹R³⁰)ₙ₅C(O)OR²⁷, —(CR²⁹R³⁰)ₙ₅C(O)NR²⁷R²⁸, —(CR²⁹R³⁰)ₙ₅S(O)₂NR²⁷R²⁸, —(CR²⁹R³⁰)ₙ₅NR²⁷R²⁸, —(CR²⁹R³⁰)ₙ₅OR²⁷, —(CR²⁹R³⁰)ₙ₅S(O)ₙ₆R²⁷, —NR³⁰C(O)NR²⁷R²⁸, —NR³⁰S(O)₂NR²⁷R²⁸ or —NR³⁰S(O)NR²⁷R²⁸ substituents, any of which may be optionally substituted;

R⁵, R⁶, R⁷, R⁸, R¹⁰, R¹¹, R¹², R¹³, R¹⁴, R¹⁵, and R¹⁶ are each independently selected from one or more of H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;

R¹⁷, R¹⁸, R¹⁹, R²⁰, R²¹, R²⁷, R²⁸, R²⁹, and R³⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;

—NR⁵R⁶ and —NR¹²R¹³ are each independently a linear structure, or, R⁵ and R⁶, or R¹² and R¹³, respectively, are taken together with the nitrogen atom to which they are attached to form a 3-12 membered saturated or unsaturated ring, wherein said ring optionally includes one or more additional heteroatoms selected from O, N, or S(O)ₘ₂;

—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-12 membered saturated or unsaturated ring, wherein said ring optionally includes one or more heteroatoms selected from O, N, or S(O)ₘ₃;

—CR¹⁹R²⁰ is a linear structure, or, R¹⁹ and R²⁰ are taken together with the carbon atom to which they are attached to form a 3-12 membered saturated or unsaturated ring, wherein said ring optionally includes one or more additional heteroatoms selected from O, N, or S(O)ₘ₄;

—NR¹⁷R¹⁸ is a linear structure, or, R¹⁷ and R⁸ are taken together with the nitrogen atom to which they are attached to form a 3-12 membered saturated or unsaturated ring, wherein said ring optionally includes one or more additional heteroatoms selected from O, N, or S(O)ₘ₅;

—CR²⁹R³⁰ is a linear structure, or, R²⁹ and R³⁰ are taken together with the carbon atom to which they are attached to form a 3-12 membered saturated or unsaturated ring, wherein said ring optionally includes one or more additional heteroatoms selected from O, N, or S(O)ₘ₆;

—NR²⁷R²⁸ is a linear structure, or, R²⁷ and R²⁸ are taken together with the nitrogen atom to which they are attached to form a 3-12 membered saturated or unsaturated ring, wherein said ring optionally includes one or more additional heteroatoms selected from O, N, or S(O)ₘ₇;

wherein m1, m2, m3, m4, m5, m6, m7, n0, n1, n2, n3, n4, n5 and n6 are each independently selected from 0, 1 or 2;

or a pharmaceutically acceptable salt, solvate or a prodrug thereof.

In some embodiments:

R¹ is selected from C₀₋₈alkyl-, C₃₋₈cycloalkyl-C₀₋₈alkyl-, or aryl-C₀₋₈alkyl-;

G¹ is selected from one or more of D, halo, —CN, —CD₃, —OCD₃, -oxo-, —CF₃, —OCF₃, —OCHF₂, —B(OH)₂, —C₀₋₈alkyl, —C₂₋₈alkenyl, —C₂₋₈alkynyl, C₃₋₈cycloalkyl-C₀₋₈alkyl-, C₃₋₈heterocycloalkyl-C₀₋₈alkyl-, aryl-C₀₋₈alkyl-, heteroaryl-C₀₋₈alkyl-, —OC₀₋₈alkyl, or —S(O)ₙ₁R¹².

In some embodiments:

G¹ is selected from 0 to 3 of D, halo, —CN, —CD₃, —OCD₃, -oxo-, —CF₃, —OCF₃, —OCHF₂, —B(OH)₂, —C₀₋₈alkyl, —C₂₋₈alkenyl, —C₂₋₈alkynyl, C₃₋₈cycloalkyl-C₀₋₈alkyl-, C₃₋₈heterocycloalkyl-C₀₋₈alkyl-, aryl-C₀₋₈alkyl-, heteroaryl-C₀₋₈alkyl-, —OC₀₋₈alkyl, or —S(O)ₙ₁R¹².

In some embodiments:

R¹ is selected from C₀₋₂alkyl-, C₄₋₆cycloalkyl-C₀₋₂alkyl-, or aryl-C₀₋₂alkyl-;

G¹ is selected from one or more of D, halo, —CN, —CD₃, —OCD₃, -oxo-, —CF₃, —OCF₃, —OCHF₂, —B(OH)₂, —C₀₋₂alkyl, —C₂₋₄alkenyl, —C₂₋₄alkynyl, C₄₋₆cycloalkyl-C₀₋₂alkyl-, C₄₋₆heterocycloalkyl-C₀₋₂alkyl-, aryl-C₀₋₃alkyl-, heteroaryl-C₀₋₂alkyl-, —OC₀₋₂alkyl, or —S(O)ₙ₁R¹².

In some embodiments:

G¹ is selected from is selected from 0 to 2 of D, halo, —CN, —CD₃, —OCD₃, -oxo-, —CF₃, —OCF₃, —OCHF₂, —B(OH)₂, —C₀₋₂alkyl, —C₂₋₄alkenyl, —C₂₋₄alkynyl, C₄₋₆cycloalkyl-C₀₋₂alkyl-, C₄₋₆heterocycloalkyl-C₀₋₂alkyl-, aryl-C₀₋₃alkyl-, heteroaryl-C₀₋₂alkyl-, —OC₀₋₂alkyl, or —S(O)ₙ₁R¹².

In some embodiments:

R² is selected from C₀₋₈alkyl-, C₃₋₈cycloalkyl-C₀₋₈alkyl-, or C₃₋₈heterocycloalkyl-C₀₋₈alkyl-;

R^{2a} is C₀₋₈alkyl-; or

R² and R^{2a} are each independently a linear structure, or, R² and R^{2a} are taken together with the carbon atom to which they are attached to form a 3-6 membered saturated or unsaturated ring;

G² and G^{2a} are each independently selected from one or more of D, halo, —CN, —CD₃, —OCD₃, -oxo-, —CF₃, —OCF₃, —OCHF₂, —C₀₋₈alkyl, —C₂₋₈alkenyl, —C₂₋₈alkynyl, C₃₋₈cycloalkyl-C₀₋₈alkyl-, or —OC₀₋₈alkyl.

In some embodiments:

G² and G^{2a} are each independently selected from 0 to 3 of D, halo, —CN, —CD₃, —OCD₃, -oxo-, —CF₃, —OCF₃, —OCHF₂, —C₀₋₈alkyl, —C₂₋₈alkenyl, —C₂₋₈alkynyl, C₃₋₈cycloalkyl-C₀₋₈alkyl-, or —OC₀₋₈alkyl.

In some embodiments:

R² is selected from C₀₋₂alkyl-, C₄₋₆cycloalkyl-C₀₋₂alkyl-, or C₄₋₆heterocycloalkyl-C₀₋₂alkyl-;

R^{2a} is C₀₋₂alkyl-; or

R² and R^{2a} are each independently a linear structure, or, R² and R^{2a} are taken together with the carbon atom to which they are attached to form a 3-6 membered saturated or unsaturated ring;

G² and G^{2a} are each independently selected from one or more of D, halo, —CN, —CD₃, —OCD₃, -oxo-, —CF₃, —OCF₃, —OCHF₂, —C₀₋₂alkyl, —C₂₋₄alkenyl, —C₂₋₄alkynyl, C₄₋₆cycloalkyl-C₀₋₂alkyl-, or —OC₀₋₂alkyl.

In some embodiments:

G² and G^{2a} are each independently selected from 0 to 2 of D, halo, —CN, —CD₃, —OCD₃, -oxo-, —CF₃, —OCF₃, —OCHF₂, —C₀₋₂alkyl, —C₂₋₄alkenyl, —C₂₋₄alkynyl, C₄₋₆cycloalkyl-C₀₋₂alkyl-, or —OC₀₋₂alkyl.

In some embodiments:

R³ is selected from —CN, C(O)NR⁷R⁸, S(O)ₙ₀R⁷R⁸, C₀₋₈alkyl, or C₃₋₈cycloalkyl-C₀₋₈alkyl-.

In some embodiments:

R³ is selected from —CN, C(O)NR⁷R⁸, S(O)ₙ₀R⁷R⁸, C₀₋₂alkyl, or C₄₋₆cycloalkyl-C₀₋₂alkyl-.

In some embodiments:

R⁴ is selected from C₀₋₈alkyl-, C₃₋₈cycloalkyl-C₀₋₈alkyl-, C₃₋₈heterocycloalkyl-C₀₋₈alkyl-, aryl-C₀₋₈alkyl-, aryl-C₃₋₈cycloalkyl-, aryl-C₃₋₈heterocycloalkyl-, heteroaryl-C₀₋₈alkyl-, heteroaryl-C₃₋₈cycloalkyl-, heteroaryl-C₃₋₈heterocycloalkyl-, or pyridine-N-oxide;

R^{4a} is selected from C₀₋₈alkyl-, C₃₋₈cycloalkyl-C₀₋₈alkyl-, aryl-C₀₋₈alkyl-;

G⁴ is selected from one or more of D, halo, —CN, —CD₃, —OCD₃, -oxo-, —CF₃, —OCF₃, —OCHF₂, —NR⁵R⁶, —C(O)NR¹²OH, —C₀₋₈alkyl, —C₂₋₈alkenyl, —C₂₋₈alkynyl, —OC₀₋₈alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

In some embodiments:

G⁴ is selected from 0 to 3 of D, halo, —CN, —CD₃, —OCD₃, -oxo-, —CF₃, —OCF₃, —OCHF₂, —NR⁵R⁶, —C(O)NR¹²OH, —C₀₋₈alkyl, —C₂₋₈alkenyl, —C₂₋₈alkynyl, —OC₀₋₈alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

In some embodiments:

R⁴ is selected from C₀₋₂alkyl-, C₄₋₆cycloalkyl-C₀₋₂alkyl-, C₄₋₆heterocycloalkyl-C₀₋₂alkyl-, aryl-C₀₋₂alkyl-, aryl-C₄₋₆cycloalkyl-, aryl-C₄₋₆heterocycloalkyl-, heteroaryl-C₀₋₂alkyl-, heteroaryl-C₄₋₆cycloalkyl-, heteroaryl-C₄₋₆heterocycloalkyl-, or pyridine-N-oxide;

R^{4a} is selected from C₀₋₂alkyl-, C₄₋₆cycloalkyl-C₀₋₂alkyl-, aryl-C₀₋₂alkyl-;

G⁴ is selected from one or more of D, halo, —CN, —CD₃, —OCD₃, -oxo-, —CF₃, —OCF₃, —OCHF₂, —NR⁵R⁶, —C(O)NR¹²OH, —C₀₋₂alkyl, —C₂₋₄alkenyl, —C₂₋₄alkynyl, —OC₀₋₂alkyl, —S(O)ₙ₁R¹², —C(O)R¹², C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

In some embodiments:

G⁴ is selected from 0 to 2 of D, halo, —CN, —CD₃, —OCD₃, -oxo-, —CF₃, —OCF₃, —OCHF₂, —NR⁵R⁶, —C(O)NR¹²OH, —C₀₋₂alkyl, —C₂₋₄alkenyl, —C₂₋₄alkynyl, —OC₀₋₂alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

In some embodiments:

R² is selected from methyl, ethyl, propyl, isopropyl, or one of the following groups:

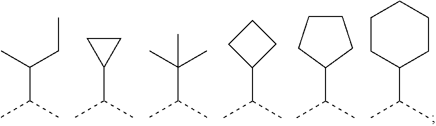

and R^{2a} is selected from H, methyl, ethyl, propyl, or isopropyl; or

R² and R^{2a} are taken together with the carbon atom to which they are attached to form one of the following groups:

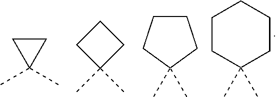

In some embodiments:

—NR⁵R⁶ and —NR¹²R¹³ are each independently a linear structure, or, R⁵ and R⁶, or R¹² and R¹³, respectively, are taken together with the nitrogen atom to which they are attached to form a 3-6 membered saturated or unsaturated ring, wherein said ring optionally includes one or more additional heteroatoms selected from O, N, or S(O)ₘ₂;

wherein m2 is selected from 0, 1 or 2.

In some embodiments:

—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-6 membered saturated or unsaturated ring, wherein said ring optionally includes one or more heteroatoms selected from O, N, or S(O)ₘ₃;

wherein m3 is selected from 0, 1 or 2.

In some embodiments:

—CR¹⁹R²⁰ is a linear structure, or, R¹⁹ and R²⁰ are taken together with the carbon atom to which they are attached to form a 3-6 membered saturated or unsaturated ring, wherein said ring optionally includes one or more additional heteroatoms selected from O, N, or S(O)ₘ₄;

wherein m4 is selected from 0, 1 or 2.

In some embodiments:

—NR¹⁷R¹⁸ is a linear structure, or, R¹⁷ and R¹⁸ are taken together with the nitrogen atom to which they are attached to form a 3-6 membered saturated or unsaturated ring, wherein said ring optionally includes one or more additional heteroatoms selected from O, N, or S(O)ₘ₅;

wherein m5 is selected from 0, 1 or 2.

In some embodiments:

—CR²⁹R³⁰ is a linear structure, or, R²⁹ and R³⁰ are taken together with the carbon atom to which they are attached to form a 3-6 membered saturated or unsaturated ring, wherein said ring optionally includes one or more additional heteroatoms selected from O, N, or S(O)ₘ₆;

wherein m6 is selected from 0, 1 or 2.

In some embodiments:

—NR²⁷R²⁸ is a linear structure, or, R²⁷ and R²⁸ are taken together with the nitrogen atom to which they are attached to form a 3-6 membered saturated or unsaturated ring, wherein said ring optionally includes one or more additional heteroatoms selected from O, N, or S(O)ₘ₇;

wherein m7 is selected from 0, 1 or 2.

In some embodiments:

R¹ is selected from one of C₆cycloalkyl-C₀₋₆alkyl-, C₆heterocycloalkyl-C₀₋₆alkyl-, 6-membered-aryl-C₀₋₆alkyl-, or 6-membered-heteroaryl-C₀₋₆alkyl-,

wherein the 4-position of R¹ is hydrogen, and wherein R¹ is optionally substituted by one or more G¹ substituents at the 2, 3, 5 and 6 positions.

In some embodiments of Formula I, compounds of the present invention are a subgenus of Formula I, having the Formula Ia:

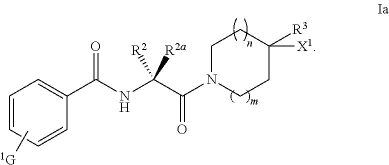

In some embodiments, the compound has the structure of Formula Id:

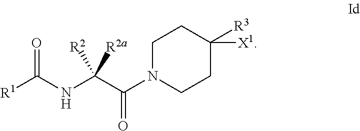

In some embodiments, R¹ is aryl substituted with one or more independent G¹ substituents (e.g., wherein the G¹ substituents are each, independently, hydrogen, halo, C₁₋₁₂alkyl, CF₃, OCF₃, OCHF₂, aryl-C₁₋₁₂alkyl, aryl, C₃₋₁₂cycloalkyl, or two G¹ substituents combine to form, with the carbons to which they are attached, an optionally substituted C₃₋₁₂cycloalkyl). In some embodiments, R¹ is 2-fluoro-3-methyl-phenyl, 2-fluoro-5-ethyl-phenyl, 2-fluoro-5-methoxy-phenyl, 2-fluoro-5-trifluoromethoxy-phenyl, 3-trifluoromethoxy-phenyl, 3-methyl-phenyl, 2-fluoro-5-trifluoromethyl-phenyl, 6-fluoro-3-methyl-2,3-dihydro-1H-indene, 2-fluoro-5-difluoromethyl-phenyl, 2-fluoro-5-tert-butyl-phenyl, 2-fluoro-5-benzyl-phenyl, 2-fluoro-5-sec-butyl-phenyl, 2-fluoro-5-phenyl-phenyl, 2-fluoro-5-cyclopropyl-phenyl, 2-fluoro-4-methyl-5-ethyl-phenyl, or 2-fluoro-5-iso-propyl-phenyl.

In some embodiments, R² is hydrogen, C₁₋₁₂alkyl (e.g., iso-propyl), or C₃₋₁₂cycloalkyl (e.g., cyclopropyl). In some embodiments, R^{2a} is hydrogen or C₁₋₁₂alkyl (e.g., iso-propyl). In some embodiments, if one of R² or R^{2a} is C₁₋₁₂alkyl (e.g., iso-propyl), or C₃₋₁₂cycloalkyl (e.g., cyclopropyl), the other is hydrogen.

In some embodiments, R³ is hydrogen, CN, C(O)NR⁷R⁸ (e.g., —C(O)NH(CH₃), —C(O)N(CH₃)₂), or C₁₋₁₂alkyl (e.g., methyl, or —CH₂OCH₃).

In some embodiments, X¹ is —(C₀₋₂alkyl)-NR⁴R^{4a} or —(C₀₋₂alkyl)-OR⁴. In some embodiments, R^{4a} is hydrogen or methyl. In some embodiments, R⁴ is aryl, aryl-C₁₋₁₂alkyl, or heteroaryl substituted with one or more independent G⁴ substituents (e.g., the G⁴ substituents are hydrogen, —CN, —OC₀₋₁₂alkyl, —NR¹²C(O)R¹³, —C(O)OR¹², or —C₀₋₁₂alkyl-S(O)ₙ₁R¹²). In some embodiments, the G⁴ substituents are hydrogen, —CN, —OCH₃, —NHC(O)CH₃, —CH₂—SO₂CH₃, —CH₂—SO₂CH₃, —C(O)OH, or —C(O)OtBu.

In some embodiments, X¹ is:

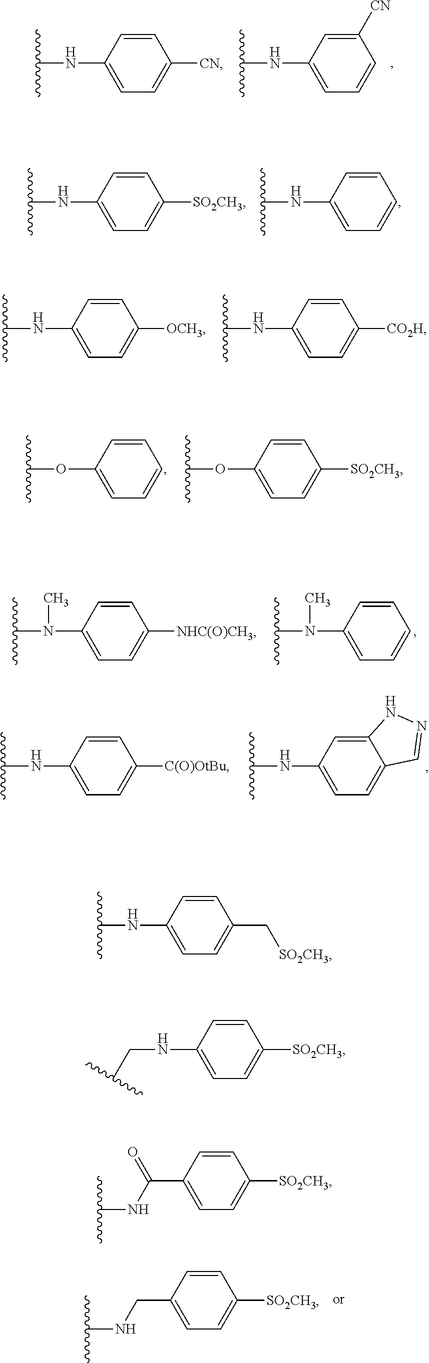

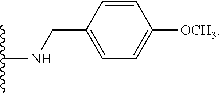 [SMILES: *NCc1ccc(OC)cc1]

In some embodiments:

m and n are each equal to 1.

In some embodiments:

X¹ is selected from C₁₋₂alkylR⁴, —(C₀₋₁alkyl)NR⁴R^{4a}, or —(C₀₋₁alkyl)OR⁴.

In some embodiments, the present invention includes a pharmaceutical composition comprising the compound or salt of any one of the compounds of Formula I, formulated with or without one or more pharmaceutical carriers.

In some embodiments, the present invention includes a method for the treatment of at least one of cancer, lymphocyte homing, chronic inflammation, neuropathic pain, fibrotic diseases, thrombosis, and cholestatic pruritus mediated at least in part by ATX comprising administering to a subject in need thereof a therapeutically effective amount of a compound or salt of the compound of Formula I.

In some embodiments, the present invention includes a method for the treatment of at least one of cancer, lymphocyte homing, chronic inflammation, neuropathic pain, fibrotic diseases, thrombosis, and cholestatic pruritus comprising administering to a subject in need thereof a therapeutically effective amount of a compound or salt of the compound of Formula I that binds to and inhibits ATX providing a reduction in LPA levels.

In some embodiments, the present invention includes a method of treating fibrosis, inflammation, cancer, angiogenesis, or pain in a mammal comprising administering a therapeutically effective amount of a compound according to Formula I, or a pharmaceutically acceptable salt thereof, to the mammal in need thereof.

In some embodiments, the present invention includes a method of treating lung fibrosis, asthma, chronic obstructive pulmonary disease (COPD), renal fibrosis, acute kidney injury, chronic kidney disease, liver fibrosis, skin fibrosis, fibrosis of the gut, breast cancer, pancreatic cancer, ovarian cancer, prostate cancer, glioblastoma, bone cancer, colon cancer, bowel cancer, head and neck cancer, melanoma, multiple myeloma, chronic lymphocytic leukemia, B cell lymphoma, T cell lymphoma, cancer pain, tumor metastasis, transplant organ rejection, scleroderma, ocular fibrosis, age related macular degeneration (AMD), diabetic retinopathy, collagen vascular disease, atherosclerosis, Raynaud's phenomenon, rheumatoid arthritis, osteoarthritis or neuropathic pain in a mammal comprising administering a therapeutically effective amount of a compound according Formula I, or a pharmaceutically acceptable salt thereof, to the mammal in need thereof.

In some embodiments, the present invention further includes administering to the mammal one or more additional therapeutically active agents selected from: corticosteroids, immunosuppressants, analgesics, anti-cancer agents, anti-inflammatories, non-steroidal anti-inflammatories, dual cyclooxygenase-1 and -2 inhibitors, cyclooxygenase-2 selective inhibitors, TNFα blockers, kinase inhibitors, chemokine receptor antagonists, bronchodilators, leukotriene receptor antagonists, leukotriene formation inhibitors, prostaglandin receptor antagonists, prostaglandin formation inhibitors, monoacylglycerol kinase inhibitors, phospholipase A1 inhibitors, phospholipase A2 inhibitors, lysophospholipase D (lysoPLD) inhibitors, autotaxin inhibitors, and LPA receptor antagonists.

In some embodiments of Formula I, compounds are present as a material in substantially pure form.

In some embodiments of Formula I, compounds are selected from any one of the Examples herein or a pharmaceutically acceptable salt thereof.

Each variable definition above includes any subset thereof and the compounds of Formula I include any combination of such variables or variable subsets.

The present invention includes the compounds and salts thereof, their physical forms, preparation of the compounds, useful intermediates, and pharmaceutical compositions and formulations thereof.

The compounds of the present invention and the term “compound” in the claims include any pharmaceutically acceptable salts or solvates, and any amorphous or crystal forms, or tautomers, whether or not specifically recited in context.

The present invention includes all isomers of the compounds. Compounds may have one or more asymmetric carbon atoms can exist as two or more stereoisomers. Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. A single compound may exhibit more than one type of isomerism.

The present invention includes any stereoisomers, even if not specifically shown, individually as well as mixtures, geometric isomers, and pharmaceutically acceptable salts thereof. Where a compound or stereocenter is described or shown without definitive stereochemistry, it is to be taken to embrace all possible individual isomers, configurations, and mixtures thereof. Thus, a material sample containing a mixture of stereoisomers would be embraced by a recitation of either of the stereoisomers or a recitation without definitive stereochemistry. Also contemplated are any cis/trans isomers or tautomers of the compounds described.

The present invention includes all stereoisomers, geometric isomers and tautomeric forms of the inventive compounds, including compounds exhibiting more than one type of isomerism, and mixtures of one or more thereof. When a tautomer of the compound of Formula I exists, the compound of Formula I of the present invention includes any possible tautomers and pharmaceutically acceptable salts thereof, and mixtures thereof, except where specifically stated otherwise.

In some embodiments, a subgenus of Formula I and above embodiments is provided, wherein the subgenus of Formula I is represented by the compound of Formula Ia:

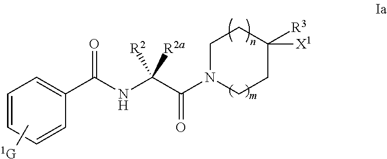

wherein R², R^{2a}, R³, G¹, X¹, m and n are as previously described for a compound of Formula I.

In some embodiments, a subgenus of Formula I and above embodiments is provided, wherein the subgenus of Formula I is represented by the compound of Formula Ib:

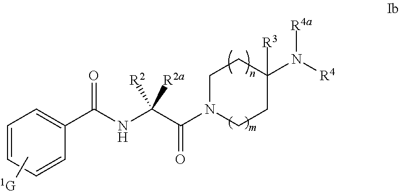

wherein R², R^{2a}, R³, R⁴, R^{4a}, G¹, m and n are as previously described for a compound of Formula I.

In some embodiments, a subgenus of Formula I and above embodiments is provided, wherein the subgenus of Formula I is represented by the compound of Formula Ic:

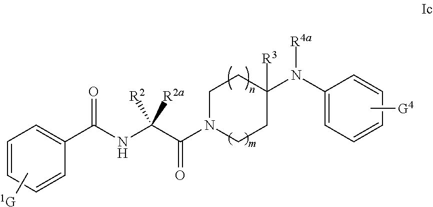

wherein R², R^{2a}, R³, R^{4a}, G¹, G⁴, m and n are as previously described for a compound of Formula I.

In some embodiments, the compound of Formula I is any one of the compounds described herein (e.g., any one of the compounds described in Examples 1 to 43)

The present invention includes the compounds, intermediates, examples and synthetic methods described herein. Compounds of Formula I are prepared according to reaction schemes described herein. Unless otherwise indicated, the substituents in the schemes are defined as above.

### Compositions:

The present invention includes pharmaceutical compositions comprising a compound or pharmaceutically acceptable salt thereof of the invention, which is formulated for a desired mode of administration with or without one or more pharmaceutically acceptable and useful carriers.

Compounds described herein may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

In general, for use in treatment, compounds described herein may be used alone, or in combination with one or more other active agents. An example of other pharmaceuticals to combine with the compounds described herein would include pharmaceuticals for the treatment of the same indication. Another example of a potential pharmaceutical to combine with compounds described herein would include pharmaceuticals for the treatment of different yet associated or related symptoms or indications. Depending on the mode of administration, compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

The compounds can also be included in pharmaceutical compositions in combination with one or more other therapeutically active compounds.

The pharmaceutical compositions of the invention comprise a compound of the invention (or a pharmaceutically acceptable salt thereof) as an active ingredient, optional pharmaceutically acceptable carrier(s) and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Compounds of the invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight.

Pharmaceutical compositions of the invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

Compositions containing a compound described by Formula I, or pharmaceutically acceptable salts thereof, may also be prepared in powder or liquid concentrate form.

### Uses:

Compounds of the present invention inhibit the activity of ATX in animals, including humans, and are useful in the treatment and/or prevention of various diseases and conditions such as cancer, lymphocyte homing and inflammation, neuropathic pain, fibrotic diseases, thrombosis, and cholestatic pruritus which are caused, mediated and/or propagated by increased LPA levels and/or the activation of ATX. In particular, compounds of the invention, and compositions thereof, are inhibitors of ATX, and are useful in treating conditions modulated, at least in part, by ATX.

In some embodiments, the invention includes a method of treating cancer comprising administering to a mammal in need thereof a therapeutically effective amount of a compound or salt of the invention.

In some embodiments, the invention includes a method of treating a cancer mediated at least in part by ATX comprising administering to a mammal in need thereof a therapeutically effective amount of a compound or salt of Formula I.

In some embodiments, the invention includes a method of treating or a method of manufacturing a medicament for treating a cancer, such as those described herein, which is mediated at least in part by ATX, comprising administering to a mammal in need thereof a therapeutically effective amount of a compound or salt of the invention.

In some embodiments, the invention includes a method of treating lymphocyte homing and inflammation comprising administering to a mammal in need thereof a therapeutically effective amount of a compound or salt of the invention.

In some embodiments, the invention includes a method of treating lymphocyte homing and inflammation mediated at least in part by ATX comprising administering to a mammal in need thereof a therapeutically effective amount of a compound or salt of Formula I.

In some embodiments, the invention includes a method of treating or a method of manufacturing a medicament for treating lymphocyte homing and inflammation, such as those described herein, which is mediated at least in part by ATX, comprising administering to a mammal in need thereof a therapeutically effective amount of a compound or salt of the invention.

In some embodiments, the invention includes a method of treating neuropathic pain comprising administering to a mammal in need thereof a therapeutically effective amount of a compound or salt of the invention.

In some embodiments, the invention includes a method of treating a neuropathic pain mediated at least in part by ATX comprising administering to a mammal in need thereof a therapeutically effective amount of a compound or salt of Formula I.

In some embodiments, the invention includes a method of treating or a method of manufacturing a medicament for treating neuropathic pain, such as those described herein, which is mediated at least in part by ATX, comprising administering to a mammal in need thereof a therapeutically effective amount of a compound or salt of the invention.

In some embodiments, the invention includes a method of treating fibrotic diseases comprising administering to a mammal in need thereof a therapeutically effective amount of a compound or salt of the invention.

In some embodiments, the invention includes a method of treating fibrotic diseases mediated at least in part by ATX comprising administering to a mammal in need thereof a therapeutically effective amount of a compound or salt of Formula I.

In some embodiments, the invention includes a method of treating or a method of manufacturing a medicament for treating a fibrotic disease, such as those described herein, which is mediated at least in part by ATX, comprising administering to a mammal in need thereof a therapeutically effective amount of a compound or salt of the invention.

In some embodiments, the invention includes a method of treating thrombosis comprising administering to a mammal in need thereof a therapeutically effective amount of a compound or salt of the invention.

In some embodiments, the invention includes a method of treating thrombosis mediated at least in part by ATX comprising administering to a mammal in need thereof a therapeutically effective amount of a compound or salt of Formula I.

In some embodiments, the invention includes a method of treating or a method of manufacturing a medicament for treating thrombosis, such as those described herein, which is mediated at least in part by ATX, comprising administering to a mammal in need thereof a therapeutically effective amount of a compound or salt of the invention.

In some embodiments, the invention includes a method of treating cholestatic pruritus comprising administering to a mammal in need thereof a therapeutically effective amount of a compound or salt of the invention.

In some embodiments, the invention includes a method of treating cholestatic pruritus mediated at least in part by ATX comprising administering to a mammal in need thereof a therapeutically effective amount of a compound or salt of Formula I.

In some embodiments, the invention includes a method of treating or a method of manufacturing a medicament for cholestatic pruritus, such as those described herein, which is mediated at least in part by ATX, comprising administering to a mammal in need thereof a therapeutically effective amount of a compound or salt of the invention.

The compounds of Formula I of the invention are useful in the treatment of a variety of cancers, including, but not limited to, solid tumors, sarcoma, fibrosarcoma, osteoma, melanoma, retinoblastoma, rhabdomyosarcoma, glioblastoma, neuroblastoma, teratocarcinoma, hematopoietic malignancy, and malignant ascites. More specifically, the cancers include, but not limited to, lung cancer, bladder cancer, pancreatic cancer, kidney cancer, gastric cancer, breast cancer, colon cancer, prostate cancer (including bone metastases), hepatocellular carcinoma, ovarian cancer, esophageal squamous cell carcinoma, melanoma, an anaplastic large cell lymphoma, an inflammatory myofibroblastic tumor, and a glioblastoma.

In some embodiments, the above methods are used to treat one or more of bladder, colorectal, non-small cell lung, breast, or pancreatic cancer. In some embodiments, the above methods are used to treat one or more of ovarian, gastric, head and neck, prostate, hepatocellular, renal, glioma, or sarcoma cancer.

In some embodiments, the invention includes a method, including the above methods, wherein the compound is used to inhibit cellular epithelial to mesenchymal transition (EMT).

In some embodiments, the method further comprises administering at least one additional active agent. In some embodiments, the invention includes a method of treating cancer comprising administering to a mammal in need thereof a therapeutically effective amount of a compound or salt of the invention, wherein at least one additional active anti-cancer agent is used as part of the method.

In some embodiments, the invention includes a method of treating the disease described herein mediated at least in part by ATX comprising administering to a mammal in need thereof a therapeutically effective regimen comprising a compound or salt of Formula I and at least one additional active agent.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the Central Nervous System (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

### DEFINITIONS

Except where otherwise indicated, the following general conventions and definitions apply. Unless otherwise indicated herein, language and terms are to be given their broadest reasonable interpretation as understood by the skilled artisan. Any examples given are nonlimiting.

Any section headings or subheadings herein are for the reader's convenience and/or formal compliance and are non-limiting.

A recitation of a compound herein is open to and embraces any material or composition containing the recited compound (e.g., a composition containing a racemic mixture, tautomers, epimers, stereoisomers, impure mixtures, etc.). In that a salt, solvate, or hydrate, polymorph, or other complex of a compound includes the compound itself, a recitation of a compound embraces materials containing such forms. Isotopically labeled compounds are also encompassed except where specifically excluded. For example, hydrogen is not limited to hydrogen containing zero neutrons.

The term “active agent” of the present invention means a compound of the invention in any salt, polymorph, crystal, solvate, or hydrated form.

The term “pharmaceutically acceptable salt(s)” is known in the art and includes salts of acidic or basic groups which can be present in the compounds and prepared or resulting from pharmaceutically acceptable bases or acids.

The term “substituted” and substitutions contained in formulas herein refer to the replacement of one or more hydrogen radicals in a given structure with a specified radical, or, if not specified, to the replacement with any chemically feasible radical. When more than one position in a given structure can be substituted with more than one substituent selected from specified groups, the substituents can be either the same or different at every position (independently selected) unless otherwise indicated. In some cases, two positions in a given structure can be substituted with one shared substituent. It is understood that chemically impossible or highly unstable configurations are not desired or intended, as the skilled artisan would appreciate.

In descriptions and claims where subject matter (e.g., substitution at a given molecular position) is recited as being selected from a group of possibilities, the recitation is specifically intended to include any subset of the recited group. In the case of multiple variable positions or substituents, any combination of group or variable subsets is also contemplated.

Unless indicated otherwise, a substituent, diradical or other group referred to herein can be bonded through any suitable position to a referenced subject molecule. For example, the term “indolyl” includes 1-indolyl, 2-indolyl, 3-indolyl, etc.

The convention for describing the carbon content of certain moieties is “(C_{a-b})” or “Cₐ-C_{b}” meaning that the moiety can contain any number of from “a” to “b” carbon atoms. C₀alkyl means a single covalent chemical bond when it is a connecting moiety, and a hydrogen when it is a terminal moiety. Similarly, “x-y” can indicate a moiety containing from x to y atoms, e.g., ₅₋₆heterocycloalkyl means a heterocycloalkyl having either five or six ring members. “C_{x-y}” may be used to define number of carbons in a group. For example, “C₀₋₁₂alkyl” means alkyl having 0-12 carbons, wherein C₀alkyl means a single covalent chemical bond when a linking group and means hydrogen when a terminal group.

The term “absent,” as used herein to describe a structural variable (e.g., “—R— is absent”) means that diradical R has no atoms, and merely represents a bond between other adjoining atoms, unless otherwise indicated.

Unless otherwise indicated (such as by a connecting “-”), the connections of compound name moieties are at the rightmost recited moiety. That is, the substituent name starts with a terminal moiety, continues with any bridging moieties, and ends with the connecting moiety. For example, “heteroarylthioC₁₋₄alkyl is a heteroaryl group connected through a sulfur to a C₁₋₄ alkyl, which alkyl connects to the chemical species bearing the substituent.

The term “aliphatic” means any hydrocarbon moiety, and can contain linear, branched, and cyclic parts, and can be saturated or unsaturated.

The term “alkyl” means any saturated hydrocarbon group that is straight-chain or branched. Examples of alkyl groups include methyl, ethyl, propyl, 2-propyl, n-butyl, iso-butyl, tert-butyl, pentyl, and the like.

The term “alkenyl” means any ethylenically unsaturated straight-chain or branched hydrocarbon group. Representative examples include, but are not limited to, ethenyl, 1-propenyl, 2-propenyl, 1-, 2-, or 3-butenyl, and the like.

The term “alkynyl” means any acetylenically unsaturated straight-chain or branched hydrocarbon group. Representative examples include, but are not limited to, ethynyl, 1-propynyl, 2-propynyl, 1-, 2-, or 3-butynyl, and the like.

The term “alkoxy” means —O-alkyl, —O-alkenyl, or —O-alkynyl. “Haloalkoxy” means an —O-(haloalkyl) group. Representative examples include, but are not limited to, trifluoromethoxy, tribromomethoxy, and the like.

“Haloalkyl” means an alkyl, preferably lower alkyl, that is substituted with one or more same or different halo atoms.

“Hydroxyalkyl” means an alkyl, preferably lower alkyl, that is substituted with one, two, or three hydroxy groups; e.g., hydroxymethyl, 1 or 2-hydroxyethyl, 1,2-, 1,3-, or 2,3-dihydroxypropyl, and the like.

The term “alkanoyl” means —C(O)-alkyl, —C(O)-alkenyl, or —C(O)-alkynyl.

“Alkylthio” means an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

The term “cyclic” means any ring system with or without heteroatoms (N, O, or S(O)₀₋₂), and which can be saturated or unsaturated. Ring systems can be bridged and can include fused rings. The size of ring systems may be described using terminology such as “_{x-y}cyclic,” which means a cyclic ring system that can have from x to y ring atoms. For example, the term “₉₋₁₀carbocyclic” means a 5,6 or 6,6 fused bicyclic carbocyclic ring system which can be saturated, unsaturated or aromatic. It also means a phenyl fused to one 5 or 6 membered saturated or unsaturated carbocyclic group. Nonlimiting examples of such groups include naphthyl, 1,2,3,4 tetrahydronaphthyl, indenyl, indanyl, and the like.

The term “carbocyclic” means a cyclic ring moiety containing only carbon atoms in the ring(s) without regard to aromaticity. A 3-10 membered carbocyclic means chemically feasible monocyclic and fused bicyclic carbocyclics having from 3 to 10 ring atoms. Similarly, a 4-6 membered carbocyclic means monocyclic carbocyclic ring moieties having 4 to 6 ring carbons, and a 9-10 membered carbocyclic means fused bicyclic carbocyclic ring moieties having 9 to 10 ring carbons.

The term “cycloalkyl” means a non-aromatic 3-12 carbon mono-cyclic, bicyclic, or polycyclic aliphatic ring moiety. Cycloalkyl can be bicycloalkyl, polycycloalkyl, bridged, or spiroalkyl. One or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like.

The term “unsaturated carbocyclic” means any cycloalkyl containing at least one double or triple bond. The term “cycloalkenyl” means a cycloalkyl having at least one double bond in the ring moiety.

The terms “bicycloalkyl” and “polycycloalkyl” mean a structure consisting of two or more cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1]heptyl (“norbornyl”), bicyclo[2.2.2]octyl, and the like.

The term “spiroalkyl” means a structure consisting of two cycloalkyl moieties that have exactly one atom in common. Examples include, but are not limited to, spiro[4.5]decyl, spiro[2.3]hexyl, and the like.

The term “aromatic” means a planar ring moieties containing 4n+2 pi electrons, wherein n is an integer.

The term “aryl” means aromatic moieties containing only carbon atoms in its ring system. Non-limiting examples include phenyl, naphthyl, and anthracenyl. The terms “aryl-alkyl” or “arylalkyl” or “aralkyl” refer to any alkyl that forms a bridging portion with a terminal aryl.

“Aralkyl” means alkyl that is substituted with an aryl group as defined above; e.g., —CH₂ phenyl, —(CH₂)₂phenyl, —(CH₂)₃ phenyl, CH₃CH(CH₃)CH₂phenyl, and the like and derivatives thereof.

The term “heterocyclic” means a cyclic ring moiety containing at least one heteroatom (N, O, or S(O)₀₋₂), including heteroaryl, heterocycloalkyl, including unsaturated heterocyclic rings.

The term “heterocycloalkyl” means a non-aromatic monocyclic, bicyclic, or polycyclic heterocyclic ring moiety of 3 to 12 ring atoms containing at least one ring having one or more heteroatoms. The rings may also have one or more double bonds. However, the rings do not have a completely conjugated pi-electron system. Examples, without limitation, of heterocycloalkyl rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocycloalkyl rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocycloalkyl rings. The term “heterocycloalkyl” also includes fused ring systems and can include a carbocyclic ring that is partially or fully unsaturated, such as a benzene ring, to form benzofused heterocycloalkyl rings. For example, 3,4-dihydro-1,4-benzodioxine, tetrahydroquinoline, tetrahydroisoquinoline and the like. The term “heterocycloalkyl” also includes heterobicycloalkyl, heteropolycycloalkyl, or heterospiroalkyl, which are bicycloalkyl, polycycloalkyl, or spiroalkyl, in which one or more carbon atom(s) are replaced by one or more heteroatoms selected from O, N, and S. For example, 2-oxa-spiro[3.3]heptane, 2,7-diaza-spiro[4.5]decane, 6-oxa-2-thia-spiro[3.4]octane, octahydropyrrolo[1,2-a]pyrazine, 7-aza-bicyclo[2.2.1]heptane, 2-oxa-bicyclo[2.2.2]octane, and the like, are such heterocycloalkyls.

Examples of saturated heterocyclic groups include, but are not limited to oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl, and 1,4-diazepanyl.

Non-aryl heterocyclic groups include saturated and unsaturated systems and can include groups having only 4 atoms in their ring system. The heterocyclic groups include benzo-fused ring systems and ring systems substituted with one or more oxo moieties. Recitation of ring sulfur is understood to include the sulfide, sulfoxide or sulfone where feasible. The heterocyclic groups also include partially unsaturated or fully saturated 4-10 membered ring systems, e.g., single rings of 4 to 8 atoms in size and bicyclic ring systems, including aromatic 6-membered aryl or heteroaryl rings fused to a non-aromatic ring. Also included are 4-6 membered ring systems (“4-6 membered heterocyclic”), which include 5-6 membered heteroaryls, and include groups such as azetidinyl and piperidinyl. Heterocyclics can be heteroatom-attached where such is possible. For instance, a group derived from pyrrole can be pyrrol-1-yl (N-attached) or pyrrol-3-yl (C-attached). Other heterocyclics include imidazo(4,5-b)pyridin-3-yl and benzoimidazol-1-yl.

Examples of heterocyclic groups include pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, and the like.

The term “unsaturated heterocyclic” means a heterocycloalkyl containing at least one unsaturated bond. The term “heterobicycloalkyl” means a bicycloalkyl structure in which at least one carbon atom is replaced with a heteroatom. The term “heterospiroalkyl” means a spiroalkyl structure in which at least one carbon atom is replaced with a heteroatom.

Examples of partially unsaturated heteroalicyclic groups include, but are not limited to: 3,4-dihydro-2H-pyranyl, 5,6-dihydro-2H-pyranyl, 2H-pyranyl, 1,2,3,4-tetrahydropyridinyl, and 1,2,5,6-tetrahydropyridinyl.

The terms “heteroaryl” or “hetaryl” mean a monocyclic, bicyclic, or polycyclic aromatic heterocyclic ring moiety containing 5-12 atoms. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. Examples include benzimidazole, benzoxazole, benzothiazole, benzofuran, quinoline, isoquinoline, quinoxaline, and the like. Furthermore, the terms “heteroaryl” include fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

Heteroaryls include, e.g., 5 and 6 membered monocyclics such as pyrazinyl and pyridinyl, and 9 and 10 membered fused bicyclic ring moieties, such as quinolinyl. Other examples of heteroaryl include quinolin-4-yl, 7-methoxy-quinolin-4-yl, pyridin-4-yl, pyridin-3-yl, and pyridin-2-yl. Other examples of heteroaryl include pyridinyl, imidazolyl, pyrimidinyl, pyrazolyl, triazolyl, pyrazinyl, tetrazolyl, furanyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. Examples of 5-6 membered heteroaryls include, thiophenyl, isoxazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl, 1,3,5-triazinyl, and the like.

“Heteroaralkyl” group means alkyl, preferably lower alkyl, that is substituted with a heteroaryl group; e.g., —CH₂ pyridinyl, —(CH₂)₂pyrimidinyl, —(CH₂)₃imidazolyl, and the like, and derivatives thereof.

A pharmaceutically acceptable heteroaryl is one that is sufficiently stable to be attached to a compound of the invention, formulated into a pharmaceutical composition and subsequently administered to a patient in need thereof.

Examples of monocyclic heteroaryl groups include, but are not limited to: pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl tetrazolyl, pyridinyl, pyridazinyl, pyrimidinyl, pyrazinyl.

Examples of fused ring heteroaryl groups include, but are not limited to: benzoduranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, azaquinazoline, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrimido[2,3-b]pyrazinyl, pyrimido[4,5-d]pyrimidinyl.

“Arylthio” means an —S-aryl or an —S-heteroaryl group, as defined herein. Representative examples include, but are not limited to, phenylthio, pyridinylthio, furanylthio, thienylthio, pyrimidinylthio, and the like and derivatives thereof.

The term “9-10 membered heterocyclic” means a fused 5,6 or 6,6 bicyclic heterocyclic ring moiety, which can be saturated, unsaturated or aromatic. The term “9-10 membered fused bicyclic heterocyclic” also means a phenyl fused to one 5 or 6 membered heterocyclic group. Examples include benzofuranyl, benzothiophenyl, indolyl, benzoxazolyl, 3H-imidazo[4,5-c]pyridin-yl, dihydrophthazinyl, 1H-imidazo[4,5-c]pyridin-1-yl, imidazo[4,5-b]pyridyl, 1,3 benzo[1,3]dioxolyl, 2H-chromanyl, isochromanyl, 5-oxo-2,3 dihydro-5H-[1,3]thiazolo[3,2-a]pyrimidyl, 1,3-benzothiazolyl, 1,4,5,6-tetrahydropyridazyl, 1,2,3,4,7,8-hexahydropteridinyl, 2-thioxo-2,3,6,9-tetrahydro-1H-purin-8-yl, 3,7-dihydro-1H-purin-8-yl, 3,4-dihydropyrimidin-1-yl, 2,3-dihydro-1,4-benzodioxinyl, benzo[1,3]dioxolyl, 2H-chromenyl, chromanyl, 3,4-dihydrophthalazinyl, 2,3-ihydro-1H-indolyl, 1,3-dihydro-2H-isoindol-2-yl, 2,4,7-trioxo-1,2,3,4,7,8-hexahydropteridin-yl, thieno[3,2-d]pyrimidinyl, 4-oxo-4,7-dihydro-3H-pyrrolo[2,3-d]pyrimidin-yl, 1,3-dimethyl-6-oxo-2-thioxo-2,3,6,9-tetrahydro-1H-purinyl, 1,2-dihydroisoquinolinyl, 2-oxo-1,3-benzoxazolyl, 2,3-dihydro-5H-1,3-thiazolo-[3,2-a]pyrimidinyl, 5,6,7,8-tetrahydro-quinazolinyl, 4-oxochromanyl, 1,3-benzothiazolyl, benzimidazolyl, benzotriazolyl, purinyl, furylpyridyl, thiophenylpyrimidyl, thiophenylpyridyl, pyrrolylpiridyl, oxazolylpyridyl, thiazolylpiridyl, 3,4-dihydropyrimidin-1-yl imidazolylpyridyl, quinoliyl, isoquinolinyl, quinazolinyl, quinoxalinyl, naphthyridinyl, pyrazolyl[3,4]pyridine, 1,2-dihydroisoquinolinyl, cinnolinyl, 2,3-dihydro-benzo[1,4]dioxin4-yl, 4,5,6,7-tetrahydro-benzo[b]-thiophenyl-2-yl, 1,8-naphthyridinyl, 1,5-napthyridinyl, 1,6-naphthyridinyl, 1,7-napthyridinyl, 3,4-dihydro-2H-1,4-benzothiazine, 4,8-dihydroxy-quinolinyl, 1-oxo-1,2-dihydro-isoquinolinyl, 4-phenyl-[1,2,3]thiadiazolyl, and the like.

The term “aryloxy” means an —O-aryl or an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

The term “oxo” means a compound containing a carbonyl group. One in the art understands that an “oxo” requires a second bond from the atom to which the oxo is attached.

The term “halo” or “halogen” means fluoro, chloro, bromo, or iodo.

“Acyl” means a —C(O)R group, where R can be selected from the nonlimiting group of hydrogen or optionally substituted lower alkyl, trihalomethyl, unsubstituted cycloalkyl, aryl, or other suitable substituent.

“Thioacyl” or “thiocarbonyl” means a —C(S)R″ group, with R as defined above.

The term “protecting group” means a suitable chemical group that can be attached to a functional group and removed at a later stage to reveal the intact functional group. Examples of suitable protecting groups for various functional groups are described in T. W. Greene and P. G. M. Wuts, Protective Groups in Organic Synthesis, 2d Ed., John Wiley and Sons (1991 and later editions); L. Fieser and M. Fieser, Fieser and Fieser's Reagents for Organic Synthesis, John Wiley and Sons (1994); and L. Paquette, ed. Encyclopedia of Reagents for Organic Synthesis, John Wiley and Sons (1995). The term “hydroxy protecting group”, as used herein, unless otherwise indicated, includes Ac, CBZ, and various hydroxy protecting groups familiar to those skilled in the art including the groups referred to in Greene.

The term “linear structure” means a moiety having substituents that do not cyclize to form a ring system. A representative example includes, but is not limited to, a compound including —NR⁵R⁶ where any atoms of “R⁵” and any atoms of “R⁶” do not connect to form a ring.

As used herein, the term “pharmaceutically acceptable salt” means those salts which retain the biological effectiveness and properties of the parent compound and do not present insurmountable safety or toxicity issues.

The term “pharmaceutical composition” means an active compound in any form suitable for effective administration to a subject, e.g., a mixture of the compound and at least one pharmaceutically acceptable carrier.

As used herein, a “physiologically/pharmaceutically acceptable carrier” means a carrier or diluent that does not cause significant irritation to an organism and does not abrogate the biological activity and properties of the administered compound.

A “pharmaceutically acceptable excipient” means an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The terms “treat,” “treatment,” and “treating” means reversing, alleviating, or inhibiting the progress of the disorder or condition to which such term applies, or one or more symptoms of such disorder or condition. “Preventing” means partially or completely treating before the disorder or condition occurs.

“Therapeutically effective amount” means that amount of the compound being administered which will relieve to some extent one or more of the symptoms of the disorder being treated, or result in inhibition of the progress or at least partial reversal of the condition.

### Synthesis and Preparation:

Compounds of the present invention include the intermediates, examples, and synthetic methods described herein.

The compounds of Formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art [such as those methods disclosed in standard reference books such as the Compendium of Organic Synthetic Methods, Vol. I-VI (Wiley-Interscience); or the Comprehensive Organic Transformations, by R. C. Larock (Wiley-Interscience)]. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed herein below and the general skill in the art. Unless otherwise indicated, the substituents in the Schemes are defined as above. Isolation and purification of the products is accomplished by standard procedures, which are known to a chemist of ordinary skill.

When a general or exemplary synthetic procedure is referred to, one skilled in the art can readily determine the appropriate reagents, if not indicated, extrapolating from the general or exemplary procedures. Some of the general procedures are given as examples for preparing specific compounds. One skilled in the art can readily adapt such procedures to the synthesis of other compounds. Representation of an unsubstituted position in structures shown or referred to in the general procedures is for convenience and does not preclude substitution as described elsewhere herein. For specific groups that can be present, either as groups in the general procedures or as optional substituents not shown, refer to the descriptions in the remainder of this document, including the claims, summary and detailed description.

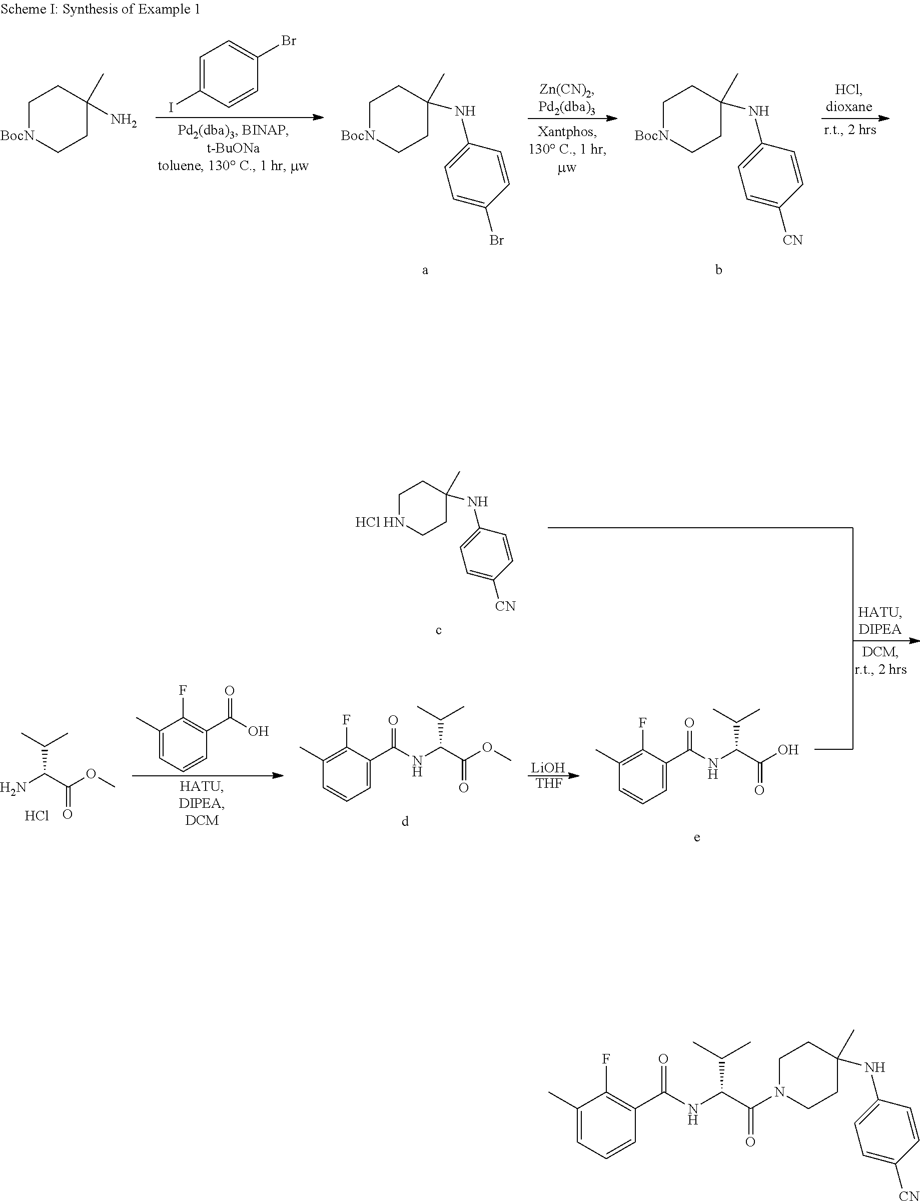

### tert-Butyl-4-(4-bromophenylamino)-4-methylpiperidine-1-carboxylate

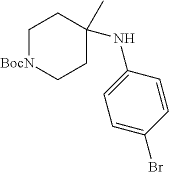 [SMILES: CC1(Nc2ccc(Br)cc2)CCN(C(=O)OC(C)(C)C)CC1]

To a solution of tert-butyl 4-amino-4-methylpiperidine-1-carboxylate (200 mg, 0.93 mmol) in toluene was added sequentially 1-bromo-4-iodobenzene (580 mg, 1.87 mmol), tris(dibenzylideneacetone)dipalladium(0) (Pd₂(dba)₃) (54 mg, 0.09 mmol), 2,2′-bis(diphenylphosphino)-1,1′-binaphthyl (BINAP) (116 mg, 0.19 mmol) and sodium tert-butoxide (270 mg, 2.8 mmol) under an argon atmosphere. The reaction mixture was heated to 130° C. for 1 hour in a microwave reactor. The solvent was removed under reduced pressure and the residue was purified by silica gel column chromatography eluting with ethyl acetate:petroleum ether=5:1 to afford tert-butyl-4-(4-bromophenylamino)-4-methylpiperidine-1-carboxylate as a brown oil (100 mg, 29%).

LCMS (ESI): m/z=369, 371 [M+H]⁺.

### tert-Butyl-4-(4-cyanophenylamino)-4-methylpiperidine-1-carboxylate

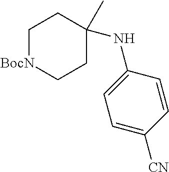 [SMILES: CC1(Nc2ccc(C#N)cc2)CCN(C(=O)OC(C)(C)C)CC1]

To a solution of tert-butyl-4-(4-bromophenylamino)-4-methylpiperidine-1-carboxylate (100 mg, 0.27 mmol) in N,N-dimethylformamide was added sequentially zinc cyanide (158 mg, 1.36 mmol), tris(dibenzylideneacetone)dipalladium(0) (Pd₂(dba)₃) (18 mg, 0.03 mmol), 4,5-bis(diphenylphosphino)-9,9-dimethylxanthene (Xantphos) (35 mg, 0.06 mmol) under an argon atmosphere. The reaction mixture was heated to 130° C. for 1 hour in a microwave reactor. After quenching with ice-water (5 mL), the reaction mixture was extracted with ethyl acetate (3×15 mL). The combined organic layers were dried over anhydrous sodium sulfate and concentrated under reduced pressure. The residue was purified by silica gel column chromatography eluting with ethyl acetate:petroleum ether=1:1 to afford tert-butyl-4-(4-cyanophenylamino)-4-methylpiperidine-1-carboxylate as a brown oil (90 mg, 100%).

LCMS (ESI): m/z=316 [M+H]⁺.

### 4-(4-Methylpiperidin-4-ylamino)benzonitrile hydrochloride

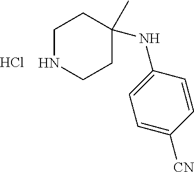 [SMILES: Cl ; CC1(Nc2ccc(C#N)cc2)CCNCC1]

A solution of tert-butyl-4-(4-cyanophenylamino)-4-methylpiperidine-1-carboxylate (90 mg, 0.00026 mol) in a 6.0 M solution of hydrochloric acid in dioxane (10 mL) was stirred for 30 minutes. The solvent was removed under reduced pressure to afford crude 4-(4-methylpiperidin-4-ylamino)benzonitrile hydrochloride (92 mg, crude), which was used directly without further purification.

LCMS (ESI): m/z=216 [M+H]⁺.

### (R)-Methyl-2-(2-fluoro-3-methylbenzamido)-3-methylbutanoate

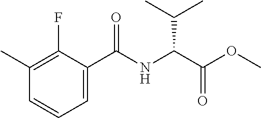

To a solution of 2-fluoro-3-methylbenzoic acid (1.54 g, 10 mmol) in dichloromethane (50 mL) was added sequentially D-valine methyl ester hydrochloride (1.71 g, 10.2 mmol), 1-[bis(dimethylamino)methylene]-1H-1,2,3-triazolo[4,5-b]pyridinium-3-oxide hexafluorophosphate (HATU) (4.2 g, 11.0 mmol) and N,N-diisopropylethylamine (3.9 g, 30 mmol). Before quenching with ice-water (50 mL), the reaction was stirred for 2 hours. The mixture was extracted with dichloromethane (3×100 mL). The combined organic layers were dried over sodium sulfate and the solvent was removed under reduced pressure. The residue was purified by silica gel column chromatography eluting with ethyl acetate:petroleum ether=1:4 to afford (R)-methyl-2-(2-fluoro-3-methylbenzamido)-3-methylbutanoate as a thick oil (3.5 g, crude), which was used directly without further purification.

LCMS (ESI): m/z=268.2 [M+H]⁺.

### (R)-2-(2-Fluoro-3-methylbenzamido)-3-methylbutanoic acid

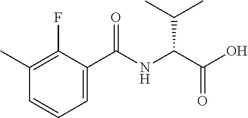 [SMILES: Cc1cccc(C(=O)N[C@@H](C(=O)O)C(C)C)c1F]

To a solution of (R)-methyl-2-(2-fluoro-3-methylbenzamido)-3-methylbutanoate (3.9 g, crude) in methanol (30 mL) was added an aqueous lithium hydroxide solution (20 mL, 2.0 M, 0.044 mol). Before quenching with ice-water (20 mL), the reaction was stirred for 1 hour. The pH of the solution was adjusted to 3 by addition of a 5% aqueous hydrochloric acid solution. The mixture was filtered and the filter cake was washed with petroleum ether (30 mL) and dried under reduced pressure to afford (R)-2-(2-fluoro-3-methylbenzamido)-3-methylbutanoic acid as a thick oil (2.40 g, 94% over two steps).

LCMS (ESI): m/z=254.1 [M+H]⁺.

¹H-NMR (300 MHz, CDCl₃): δ=1.05 (t, J=7.8 Hz, 6H), 2.32 (s, 3H), 2.34-2.44 (m, 1H), 4.78-4.82 (m, 1H), 7.06-7.13 (m, 1H), 7.27-7.35 (m, 1H), 7.79-7.91 (m, 1H).

### Example 1

### (R)—N-(1-(4-(4-Cyanophenylamino)-4-methylpiperidin-1-yl)-3-methyl-1-oxobutan-2-yl)-2-fluoro-3-methylbenzamide

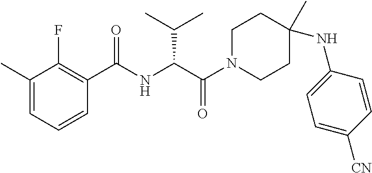 [SMILES: Cc1cccc(C(=O)N[C@@H](C(=O)N2CCC(C)(Nc3ccc(C#N)cc3)CC2)C(C)C)c1F]

To a suspension of 4-(4-methylpiperidin-4-ylamino)benzonitrile hydrochloride (92 mg, crude) in dichloromethane (5 mL) was added (R)-2-(2-fluoro-3-methylbenzamido)-3-methylbutanoic acid (100 mg, 0.394 mmol), 1-[bis(dimethylamino)methylene]-1H-1,2,3-triazolo[4,5-b]pyridinium-3-oxide hexafluorophosphate (HATU) (204 mg, 0.538 mmol) and N,N-diisopropylethylamine (139 mg, 1.076 mmol). The reaction was stirred for 2 hours before quenching with ice-water. The mixture was extracted with ethyl acetate (3×10 mL) and the combined organic layers were dried over anhydrous sodium sulfate and concentrated under reduced pressure. The residue was purified by prep-TLC with dichloromethane:methanol=15:1 to afford (R)—N-(1-(4-(4-cyanophenylamino)-4-methylpiperidin-1-yl)-3-methyl-1-oxobutan-2-yl)-2-fluoro-3-methylbenzamide (Example 1) as a white solid (30 mg, 23%).

LCMS (ESI): m/z=451.2 [M+H]⁺.

¹H-NMR (400 MHz, CDCl₃): δ=0.98-1.06 (m, 6H), 1.27-1.30 (m, 3H), 1.62-1.77 (m, 2H), 2.11-2.19 (m, 3H), 2.38-2.39 (m, 3H), 3.17-3.23 (m, 1H), 3.42-4.31 (m, 3H), 4.95-4.97 (m, 1H), 6.82-7.56 (m, 6H), 8.25-8.27 (m, 1H).

### Example 2

### Prepared Using a Method Analogous to Example 1

### (R)—N-(1-(4-(3-Cyanophenylamino)-4-methylpiperidin-1-yl)-3-methyl-1-oxobutan-2-yl)-2-fluoro-3-methylbenzamide

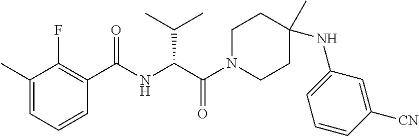 [SMILES: Cc1cccc(C(=O)N[C@@H](C(=O)N2CCC(C)(Nc3cccc(C#N)c3)CC2)C(C)C)c1F]

10 mg, yield: 12%, appearance: white solid.

LCMS (ESI): m/z=451.2 [M+H]⁺.

¹H-NMR (400 MHz, CD₃OD): δ=0.93-1.11 (m, 6H), 1.42-1.45 (m, 3H), 1.61-1.88 (m, 2H), 1.89-2.28 (m, 3H), 2.35 (s, 3H), 3.40-3.49 (m, 1H), 3.50-3.77 (m, 1H), 3.92-4.15 (m, 2H), 4.85-4.96 (m, 1H), 6.97-7.68 (m, 8H).

### Example 3

### Prepared Using a Method Analogous to Example 1

### (R)-2-Fluoro-3-methyl-N-(3-methyl-1-(4-methyl-4-(4-(methylsulfonyl)phenylamino)piperidin-1-yl)-1-oxobutan-2-yl)benzamide

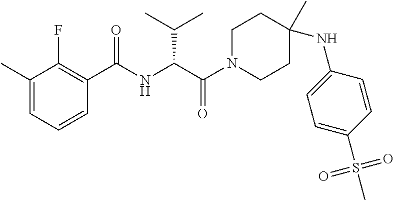 [SMILES: Cc1cccc(C(=O)N[C@@H](C(=O)N2CCC(C)(Nc3ccc(S(C)(=O)=O)cc3)CC2)C(C)C)c1F]

12.3 mg, yield: 37%, appearance: white solid.

LCMS (ESI): m/z=504.2 [M+H]⁺.

¹H-NMR (400 MHz, CD₃OD): δ=1.02-1.09 (m, 6H), 1.24-1.40 (m, 3H), 1.63-1.77 (m, 2H), 2.10-2.17 (m, 3H), 2.33-2.34 (m, 3H), 3.04 (s, 3H), 3.21-4.19 (m, 4H), 4.96-4.98 (m, 1H), 6.92-7.66 (m, 7H).

### Example 4

### Prepared Using a Method Analogous to Example 1

### (R)-5-Ethyl-2-fluoro-N-(3-methyl-1-(4-methyl-4-(4-(methylsulfonyl)phenylamino)piperidin-1-yl)-1-oxobutan-2-yl)benzamide

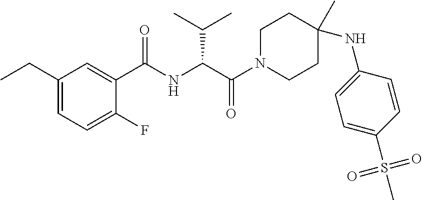 [SMILES: CCc1ccc(F)c(C(=O)N[C@@H](C(=O)N2CCC(C)(Nc3ccc(S(C)(=O)=O)cc3)CC2)C(C)C)c1]

2.8 mg, yield: 14%, appearance: white solid.

LCMS (ESI): m/z=517.8 [M+H]⁺.

¹H-NMR (400 MHz, CD₃OD): δ=1.00-1.06 (m, 6H), 1.22-1.31 (m, 3H), 1.46-1.50 (m, 3H), 1.63-1.82 (m, 2H), 2.13-2.29 (m, 3H), 2.65-2.73 (m, 2H), 3.03-3.04 (m, 3H), 3.15-3.22 (m, 1H), 3.42-3.58 (m, 1H), 3.67-3.72 (m, 1H), 3.84-4.01 (m, 1H), 4.17-4.20 (m, 1H), 4.87-4.98 (m, 1H), 6.89-6.92 (m, 2H), 7.11-7.15 (m, 1H), 7.39-7.40 (m, 1H), 7.58-7.65 (m, 3H), 8.18-8.20 (m, 1H).

### Example 5

### Prepared Using a Method Analogous to Example 1

### (R)-2-Fluoro-5-methoxy-N-(3-methyl-1-(4-methyl-4-(4-(methylsulfonyl)phenylamino)piperidin-1-yl)-1-oxobutan-2-yl)benzamide

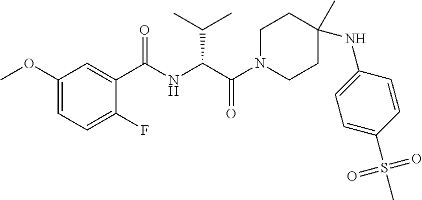 [SMILES: COc1ccc(F)c(C(=O)N[C@@H](C(=O)N2CCC(C)(Nc3ccc(S(C)(=O)=O)cc3)CC2)C(C)C)c1]

23.2 mg, yield: 37%, appearance: white solid.

LCMS (ESI): m/z=520.2 [M+H]⁺.

¹H-NMR (400 MHz, CD₃OD): δ=1.00-1.06 (m, 6H), 1.26-1.36 (m, 1H), 1.45-1.50 (d, 3H), 1.64-1.85 (m, 2H), 2.12-2.29 (m, 3H), 3.04 (m, 3H), 3.21-3.59 (m, 1H), 3.65-3.78 (m, 1H), 3.81-3.84 (d, 3H), 3.92-4.18 (m, 2H), 4.97-4.99 (t, 1H), 6.91 (m, 2H), 7.08-7.20 (m, 2H), 7.26-7.31 (m, 1H), 7.63-7.66 (m, 2H).

### Example 6

### Prepared Using a Method Analogous to Example 1

### (R)-2-Fluoro-N-(3-methyl-1-(4-methyl-4-(4-(methylsulfonyl)phenylamino)piperidin-1-yl)-1-oxobutan-2-yl)-5-(trifluoromethoxy)benzamide

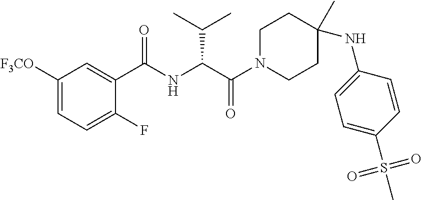 [SMILES: CC(C)[C@@H](NC(=O)c1cc(OC(F)(F)F)ccc1F)C(=O)N1CCC(C)(Nc2ccc(S(C)(=O)=O)cc2)CC1]

42.4 mg, yield: 67%, appearance: white solid.

LCMS (ESI): m/z=573.6 [M+H]⁺.

¹H-NMR (400 MHz, CD₃OD): δ=1.00-1.05 (m, 6H), 1.46-1.50 (m, 3H), 1.64-1.85 (m, 2H), 2.13-2.28 (m, 3H), 3.04 (m, 3H), 3.21-3.59 (m, 2H), 3.68-4.19 (m, 4H), 4.95-4.97 (m, 1H), 6.91-6.94 (m, 2H), 7.34-7.41 (m, 1H), 7.50-7.51 (m, 1H), 7.63-7.66 (m, 3H).

### Example 7

### Prepared Using a Method Analogous to Example 1

### (R)—N-(3-Methyl-1-(4-methyl-4-(phenylamino)piperidin-1-yl)-1-oxobutan-2-yl)-3-(trifluoromethoxy)benzamide

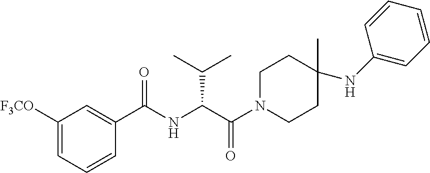 [SMILES: CC(C)[C@@H](NC(=O)c1cccc(OC(F)(F)F)c1)C(=O)N1CCC(C)(Nc2ccccc2)CC1]

47.8 mg, yield: 65%, white solid.

LCMS (ESI): m/z=478.2 [M+H]⁺.

¹H-NMR (400 MHz, CD₃OD): δ=7.88 (t, J=7.2 Hz, 1H), 7.79 (s, 1H), 7.64-7.59 (m, Hz, 1H), 7.50 (d, J=7.5 Hz, 1H), 7.17-7.11 (m, Hz, 2H), 6.85 (d, J=8.1 Hz, 2H), 6.75 (t, J=7.4 Hz, 1H), 4.07-3.39 (m, 1H), 3.89-3.38 (m, 4H), 2.25-2.20 (m, 1H), 2.11-2.00 (m, 2H), 1.75-1.54 (m, 2H), 1.35 (d, J=18.9 Hz, 3H), 1.08-0.89 (m, 6H).

### Example 8

### Prepared Using a Method Analogous to Example 1

### (R)—N-(1-(4-((4-Methoxyphenyl)amino)-4-methylpiperidin-1-yl)-3-methyl-1-oxobutan-2-yl)-3-(trifluoromethoxy)benzamide

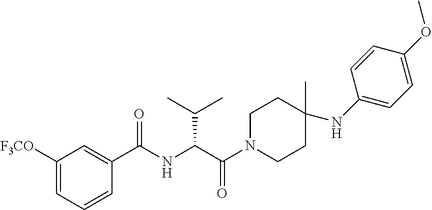 [SMILES: COc1ccc(NC2(C)CCN(C(=O)[C@H](NC(=O)c3cccc(OC(F)(F)F)c3)C(C)C)CC2)cc1]

66.1 mg, yield: 68%, white solid.

LCMS (ESI): m/z=508.2 [M+H]⁺.

¹H-NMR (400 MHz, DMSO-d₆): δ=8.79 (t, J=7.4 Hz, 1H), 7.98 (d, J=7.6 Hz, 1H), 7.89 (s, 1H), 7.71-7.46 (m, 2H), 6.83-6.65 (m, 3H), 4.81-4.69 (m, 1H), 4.51 (s, 1H), 3.87-3.58 (m, 5H), 3.47-3.37 (m, 2H), 2.25-2.12 (m, 1H), 1.93-1.73 (m, 2H), 1.53-1.31 (m, 2H), 1.27-1.10 (m, 3H), 1.03-0.80 (m, 6H).

### Example 9

### Prepared Using a Method Analogous to Example 1

### (R)-4-((4-Methyl-1-(3-methyl-2-(3-(trifluoromethoxy)benzamido)butanoyl)piperidin-4-yl)amino)benzoic acid

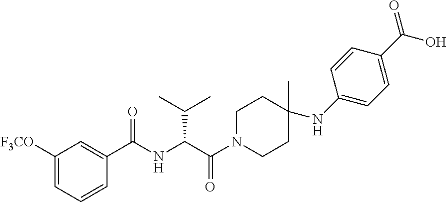 [SMILES: CC(C)[C@@H](NC(=O)c1cccc(OC(F)(F)F)c1)C(=O)N1CCC(C)(Nc2ccc(C(=O)O)cc2)CC1]

67.9 mg, yield: 53%, white solid.

LCMS (ESI): m/z=522.2 [M+H]⁺.

¹H-NMR (400 MHz, DMSO-d₆): δ=12.02 (s, 1H), 8.79 (dd, J=11.8, 8.5 Hz, 1H), 7.97 (dd, J=7.6, 6.3 Hz, 1H), 7.88 (d, J=8.9 Hz, 1H), 7.67-7.51 (m, 4H), 6.75 (t, J=8.5 Hz, 2H), 6.09 (d, J=6.3 Hz, 1H), 4.74 (dd, J=16.6, 8.3 Hz, 1H), 3.85 (dd, J=41.9, 13.4 Hz, 2H), 3.52 (dd, J=29.0, 10.6 Hz, 1H), 3.21 (dd, J=21.6, 10.7 Hz, 1H), 2.18 (dd, J=14.7, 8.0 Hz, 1H), 2.08-1.94 (m, 2H), 1.61 (br, 1H), 1.50 (t, J=9.9 Hz, 1H), 1.36 (d, J=19.1 Hz, 3H), 0.96-0.89 (m, 6H).

### Example 10

### Prepared Using a Method Analogous to Example 1

### (R)-2-Fluoro-N-(3-methyl-1-(4-methyl-4-(phenylamino)piperidin-1-yl)-1-oxobutan-2-yl)-5-(trifluoromethoxy)benzamide

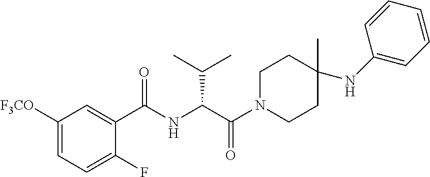 [SMILES: CC(C)[C@@H](NC(=O)c1cc(OC(F)(F)F)ccc1F)C(=O)N1CCC(C)(Nc2ccccc2)CC1]

69 mg, yield: 72%, white solid.

LCMS (ESI): m/z=496.2 [M+H]⁺.

¹H-NMR (400 MHz, CD₃OD): δ=7.71-7.61 (m, 1H), 7.56-7.45 (m, 1H), 7.44-7.31 (m, 1H), 7.14 (m, 2H), 6.91-6.80 (m, 2H), 6.76 (td, J=7.3, 4.1 Hz, 1H), 4.99 (d, J=7.1 Hz, 1H), 4.05 (dt, J=12.7, 4.2 Hz, 0.5H), 3.94-3.76 (m, 2H), 3.66 (m, 1H), 3.43 (ddd, J=13.6, 10.7, 3.2 Hz, 0.5H), 2.25-2.06 (m, 2H), 2.01 (d, J=13.9 Hz, 1H), 1.80-1.55 (m, 2H), 1.35 (d, J=15.5 Hz, 3H), 1.11-0.94 (m, 6H).

### Example 11

### Prepared Using a Method Analogous to Example 1

### (R)-4-((1-(2-(2-Fluoro-5-(trifluoromethoxy)benzamido)-3-methylbutanoyl)-4-methylpiperidin-4-yl)amino)benzoic acic

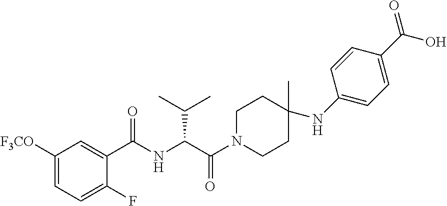 [SMILES: CC(C)[C@@H](NC(=O)c1cc(OC(F)(F)F)ccc1F)C(=O)N1CCC(C)(Nc2ccc(C(=O)O)cc2)CC1]

32.9 mg, yield: 42%, white solid.

LCMS (ESI): m/z=540.2 [M+H]⁺.

¹H-NMR (400 MHz, DMSO-d₆): δ=12.02 (s, 1H), 8.68-8.59 (m, 1H), 7.68-7.41 (m, 5H), 6.78-6.75 (m, 2H), 6.11 (d, J=2.7 Hz, 1H), 4.83-4.76 (m, 1H), 3.95-3.74 (m, 2H), 3.55-3.43 (m, 1H), 3.28-3.16 (m, 1H), 2.12-1.97 (m, 3H), 1.63 (s, 1H), 1.52 (t, J=10.1 Hz, 1H), 1.37 (d, J=14.5 Hz, 3H), 0.95-0.90 (m, 6H).

### Example 12

### Prepared Using a Method Analogous to Example 1

### (R)-2-Fluoro-N-(1-(4-((4-methoxyphenyl)amino)-4-methylpiperidin-1-yl)-3-methyl-1-oxobutan-2-yl)-5-(trifluoromethoxy)benzamide

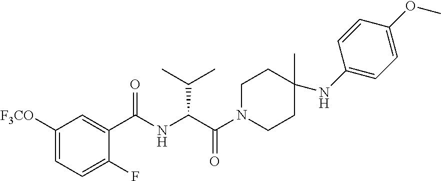 [SMILES: COc1ccc(NC2(C)CCN(C(=O)[C@H](NC(=O)c3cc(OC(F)(F)F)ccc3F)C(C)C)CC2)cc1]

120.4 mg, yield: 68%, white solid.

LCMS (ESI): m/z=526.2 [M+H]⁺.

¹H-NMR (400 MHz, CD₃OD): δ=7.71-7.61 (m, 1H), 7.56-7.46 (m, 1H), 7.38 (td, J=9.6, 1.9 Hz, 1H), 6.99-6.87 (m, 2H), 6.88-6.77 (m, 2H), 4.98 (d, J=7.2 Hz, 1H), 4.02-3.78 (m, 2H), 3.76 (d, J=3.4 Hz, 3H), 3.74-3.48 (m, 2H), 2.18 (dq, J=13.5, 6.7 Hz, 1H), 1.98-1.54 (m, 4H), 1.24 (d, J=23.2 Hz, 3H), 1.03 (d, J=6.7 Hz, 6H).

### Example 13

### Prepared Using a Method Analogous to Example 1

### (R)—N-(1-Cyclopropyl-2-(4-methyl-4-((4-(methylsulfonyl)phenyl)amino)piperidin-1-yl)-2-oxoethyl)-2-fluoro-5-(trifluoromethoxy)benzamide

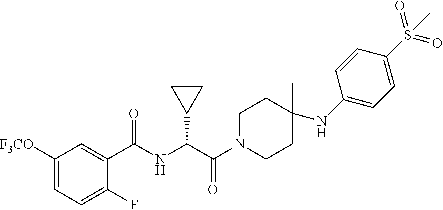 [SMILES: CC1(Nc2ccc(S(C)(=O)=O)cc2)CCN(C(=O)[C@H](NC(=O)c2cc(OC(F)(F)F)ccc2F)C2CC2)CC1]

89.4 mg, yield: 63%, white solid.

LCMS (ESI): m/z=572.2 [M+H]⁺.

¹H-NMR (400 MHz, CD₃OD): δ=7.74-7.66 (m, 1H), 7.63 (dd, J=8.9, 5.1 Hz, 2H), 7.54-7.44 (m, 1H), 7.37 (td, J=9.5, 6.7 Hz, 1H), 6.90 (dd, J=8.9, 4.0 Hz, 2H), 4.72 (dd, J=13.5, 8.3 Hz, 1H), 4.21-3.95 (m, 1H), 3.84 (m, 1H), 3.70-3.44 (m, 1H), 3.35-3.15 (m, 1H), 3.04 (d, J=3.0 Hz, 3H), 2.21 (m, 2H), 1.90-1.60 (m, 2H), 1.46 (d, J=14.5 Hz, 3H), 1.35-1.23 (m, 1H), 0.69-0.40 (m, 4H).

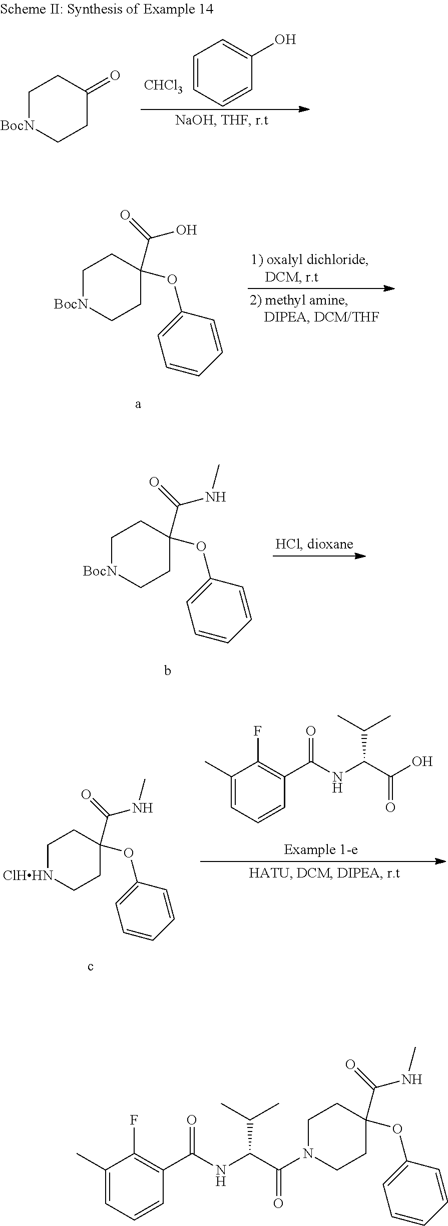

### 1-(tert-Butoxycarbonyl)-4-phenoxypiperidine-4-carboxylic acid

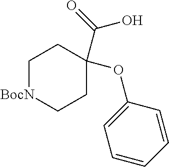 [SMILES: CC(C)(C)OC(=O)N1CCC(Oc2ccccc2)(C(=O)O)CC1]

To a solution of phenol (10.0 g, 0.106 mol) in tetrahydrofuran was added sodium hydroxide (21 g, 0.533 mol) at 0° C. After stirring for 10 minutes at 0° C., to the resulting solution was added tert-butyl 4-oxopiperidine-1-carboxylate (42 g, 0.233 mol). The resulting suspension was stirred for 20 minutes at 0° C. To the suspension was added chloroform (62.8 g, 0.532 mol). The reaction was stirred overnight before quenching with water (10 mL). The resulting mixture was extracted with ethyl acetate (100 mL). The pH of aqueous layer was adjusted to pH 3 by adding acetic acid and extracted with ethyl acetate (3×100 mL). The combined organic layers were dried over anhydrous sodium sulfate and the solvent was removed under reduced pressure to afford crude 1-(tert-butoxycarbonyl)-4-phenoxypiperidine-4-carboxylic acid (30.8 g, crude), which was used directly without further purification.

LCMS (ESI): m/z=322.2 [M+H]⁺.

### tert-Butyl-4-(methylcarbamoyl)-4-phenoxypiperidine-1-carboxylate

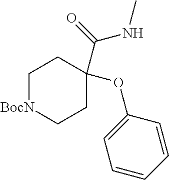 [SMILES: CNC(=O)C1(Oc2ccccc2)CCN(C(=O)OC(C)(C)C)CC1]

To a solution of 1-(tert-butoxycarbonyl)-4-phenoxypiperidine-4-carboxylic acid (200 mg, 0.623 mmol) in dichloromethane (5 mL) was added sequentially N,N-dimethylformamide (1drop, cat.), oxalyl dichloride (86 mg, 0.64 mmol) at 0° C. The resulting solution was stirred for 1 hour and the solvent was removed under reduced pressure. The residue was dissolved in dichloromethane (10 mL). To the solution was added N,N-diisopropylethylamine (240 mg, 1.87 mmol) at 0° C. After stirring for 5 minutes at 0° C., a 2.0 M solution of methylamine in tetrahydrofuran (0.47 mL 0.934 mmol) was added to the solution. The resulting mixture was stirred overnight. The reaction was quenched with brine and extracted with ethyl acetate (3×20 mL). The combined organic layers were dried over anhydrous sodium sulfate and concentrated under reduced pressure. The residue was purified by silica gel column chromatography eluting with ethyl acetate:petroleum ether=1:3 to afford tert-butyl-4-(methylcarbamoyl)-4-phenoxypiperidine-1-carboxylate (110 mg, 52%) as a colorless oil.

LCMS (ESI): m/z=335.2 [M+H]⁺.

¹H-NMR (400 MHz, CDCl₃): δ=1.56 (s, 9H), 1.95-2.21 (m, 4H), 2.88 (s, 3H), 2.99-3.09 (m, 2H), 3.87-4.01 (m, 2H), 6.33-6.54 (br, 1H), 6.85-6.90 (m, 2H), 7.05 (d, J=7.4 Hz, 1H), 7.23-7.34 (m, 3H).

### N-Methyl-4-phenoxypiperidine-4-carboxamide hydrochloride

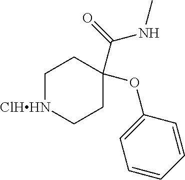

A solution of tert-butyl-4-(methylcarbamoyl)-4-phenoxypiperidine-1-carboxylate (110 mg, 0.33 mmol) in a 6.0 M solution of hydrochloric acid in dioxane (10 mL) was stirred for 30 minutes. The solvent was removed under reduced pressure to afford N-methyl-4-phenoxypiperidine-4-carboxamide hydrochloride (100 mg, crude), which was used directly without further purification.

### Example 14

### (R)-1-(2-(2-Fluoro-3-methylbenzamido)-3-methylbutanoyl)-N-methyl-4-phenoxypiperidine-4-carboxamide

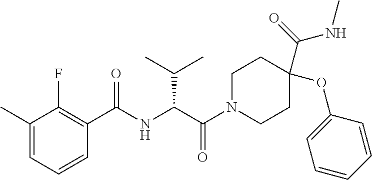 [SMILES: CNC(=O)C1(Oc2ccccc2)CCN(C(=O)[C@H](NC(=O)c2cccc(C)c2F)C(C)C)CC1]

To a suspension of N-methyl-4-phenoxypiperidine-4-carboxamide hydrochloride (100 mg, crude) in dichloromethane (5 mL) was added sequentially (R)-2-(2-fluoro-3-methylbenzamido)-3-methylbutanoic acid (prepared as described in Example 1-e) (103 mg, 0.41 mmol), 1-[bis(dimethylamino)methylene]-1H-1,2,3-triazolo[4,5-b]pyridinium-3-oxide hexafluorophosphate (HATU) (211 mg, 0.56 mmol) and N,N-diisopropylethylamine (143 mg, 1.11 mmol). The reaction was stirred for 2 hours before quenching with ice-water (10 mL). The mixture was extracted with ethyl acetate (3×10 mL) and the combined organic layers were dried over anhydrous sodium sulfate and concentrated under reduced pressure. The residue was purified by prep-TLC with dichloromethane: methanol=15:1 to afford (R)-1-(2-(2-fluoro-3-methylbenzamido)-3-methylbutanoyl)-N-methyl-4-phenoxypiperidine-4-carboxamide (Example 14) (20 mg, 12% over two steps) as a white solid.

LCMS (ESI): m/z=470.2 [M+H]⁺.

¹H-NMR (400 MHz, CD₃OD): δ=0.94-1.12 (m, 6H), 1.63-1.82 (m, 3H), 2.06-2.11 (m, 3H), 2.34 (s, 3H), 2.81-2.92 (m, 3H), 2.94-3.12 (m, 1H), 3.32-3.52 (m, 1H), 3.87-3.98 (m, 1H), 4.30-4.47 (m, 1H), 5.06-5.16 (m, 1H), 6.40-6.50 (m, 1H), 6.87-7.92 (m, 8H).

### Example 15

### Prepared Using a Method Analogous to Example 14

### (R)-1-(2-(5-Ethyl-2-fluorobenzamido)-3-methylbutanoyl)-N-methyl-4-(4-(methylsulfonyl)phenoxy)piperidine-4-carboxamide

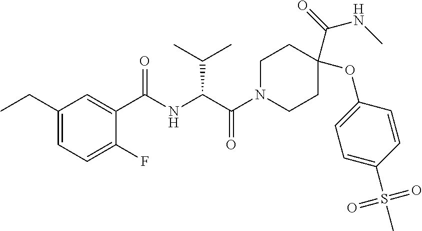 [SMILES: CCc1ccc(F)c(C(=O)N[C@@H](C(=O)N2CCC(Oc3ccc(S(C)(=O)=O)cc3)(C(=O)NC)CC2)C(C)C)c1]

82 mg, yield: 65%, appearance: white solid.

LCMS (ESI): m/z=562.0 [M+H]⁺.

¹H-NMR (400 MHz, CD₃OD): δ=0.99-1.06 (m, 6H), 1.23-1.28 (m, 3H), 2.08-2.33 (m, 5H), 2.66-2.78 (m, 5H), 3.03-3.20 (m, 4H), 3.48-3.61 (m, 1H), 4.10-4.43 (m, 2H), 4.95-4.98 (m, 1H), 7.11-8.42 (m, 8H).

### Example 16

### Prepared Using a Method Analogous to Example 14

### (R)-1-(2-(5-Ethyl-2-fluorobenzamido)-3-methylbutanoyl)-N,N-dimethyl-4-(4-(methylsulfonyl)phenoxy)piperidine-4-carboxamide

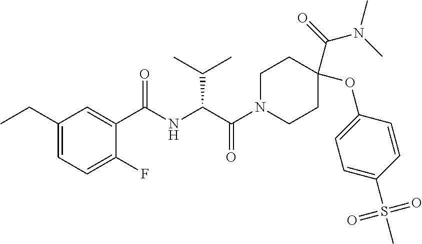 [SMILES: CCc1ccc(F)c(C(=O)N[C@@H](C(=O)N2CCC(Oc3ccc(S(C)(=O)=O)cc3)(C(=O)N(C)C)CC2)C(C)C)c1]

53 mg, yield: 41%, appearance: white solid.

LCMS (ESI): m/z=576.3 [M+H]⁺.

¹H-NMR (400 MHz, CD₃OD): δ=1.00-1.06 (m, 6H), 1.23-1.28 (m, 3H), 2.13-2.46 (m, 5H), 2.66-2.73 (m, 2H), 2.96-2.99 (m, 3H), 3.12-3.18 (m, 7H), 3.50-3.70 (m, 1H), 4.07-4.40 (m, 2H), 4.96-4.98 (m, 1H), 7.11-7.94 (m, 7H).

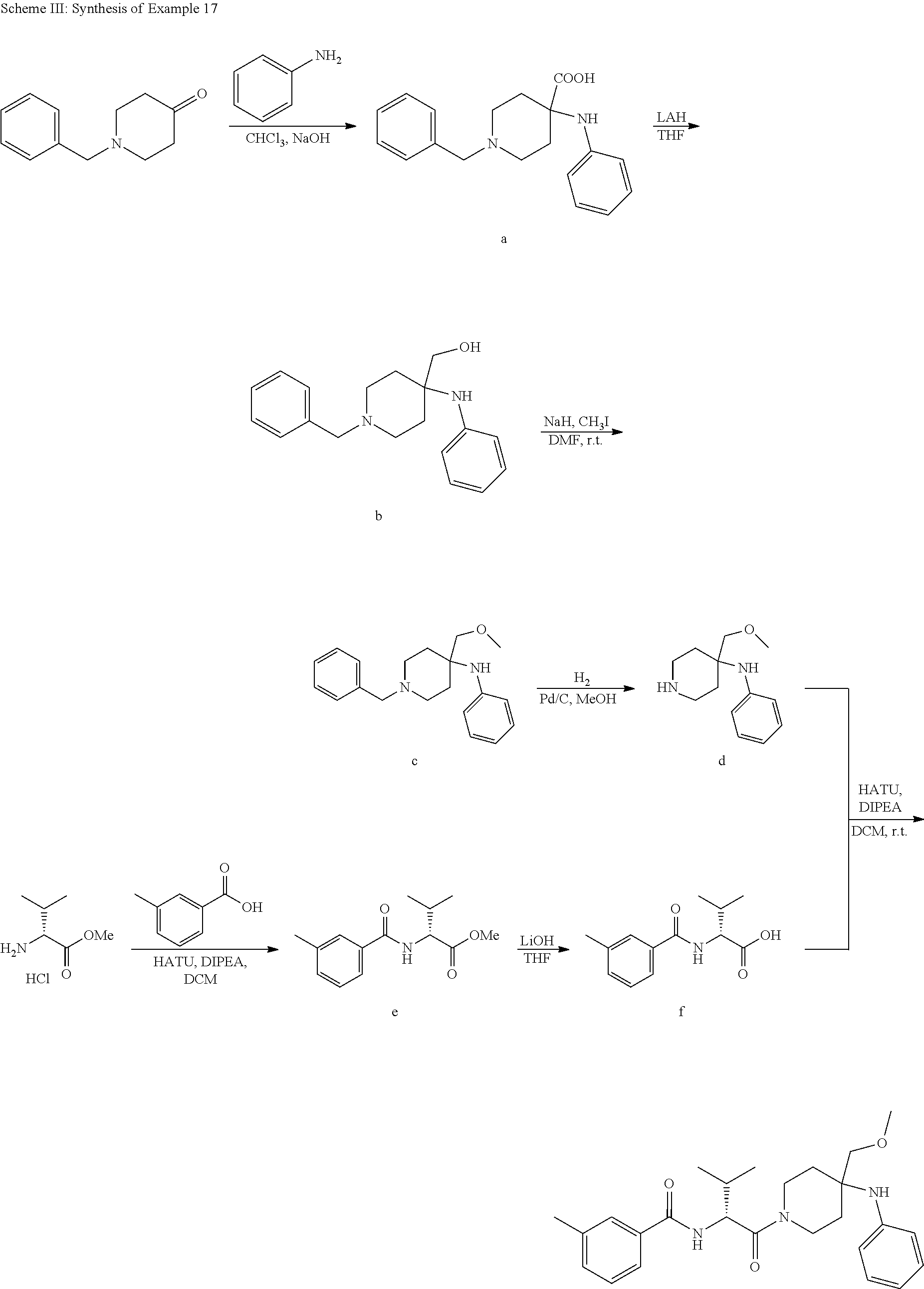

### 1-Benzyl-4-(phenylamino)piperidine-4-carboxylic acid

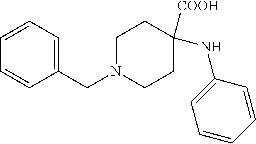 [SMILES: O=C(O)C1(Nc2ccccc2)CCN(Cc2ccccc2)CC1]

To a solution of aniline (2 g, 21 mmol) in tetrahydrofuran was added sodium hydroxide (4.3 g, 107 mmol) at 0° C. After stirring for 10 minutes at 0° C., to the resulting solution was added tert-butyl 4-oxopiperidine-1-carboxylate (43 g, 21.6 mmol). The resulting suspension was stirred for 20 minutes at 0° C. To the suspension was added chloroform (12.7 g, 107 mmol). The reaction was stirred overnight before quenching with water (10 mL). The resulting mixture was extracted with ethyl acetate (100 mL). The pH of aqueous layer was adjusted to pH 3 by addition of 5% aqueous hydrochloride solution and extracted with ethyl acetate (3×100 mL). The combined organic layers were dried over anhydrous sodium sulfate and the solvent was removed under reduced pressure to afford 1-benzyl-4-(phenylamino)piperidine-4-carboxylic acid as a brown solid (0.38 g, 8%).

LCMS (ESI): m/z=311.2 [M+H]⁺.

¹H-NMR (300 MHz, DMSO-d₆): δ=2.18-2.28 (m, 4H), 2.96-3.25 (m, 4H), 4.25-4.35 (m, 2H), 6.48-6.68 (m, 3H), 7.04-7.18 (m, 2H), 7.40-7.50 (m, 3H), 7.56-773 (m, 2H).

### (1-Benzyl-4-(phenylamino)piperidin-4-yl)methanol

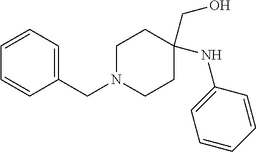 [SMILES: OCC1(Nc2ccccc2)CCN(Cc2ccccc2)CC1]

To a solution of 1-benzyl-4-(phenylamino)piperidine-4-carboxylic acid (300 mg, 0.94 mmol) in tetrahydrofuran (10 mL) was added a 2.4 M solution of lithium aluminum hydride in tetrahydrofuran (0.8 mL, 1.93 mmol). Before quenching with ice-water (1 mL), the reaction was stirred for 3 hours at 70° C. The resulting suspension was filtered and the filtrate was concentrated under reduced pressure. The residue was purified by silica gel column chromatography eluting with ethyl acetate:petroleum ether=1:1 to afford (1-benzyl-4-(phenylamino)piperidin-4-yl)methanol as a yellow oil (45 mg, crude), which was used directly without further purification.

LCMS (ESI): m/z=297.1 [M+H]⁺.

### 1-Benzyl-4-(methoxymethyl)-N-phenylpiperidin-4-amine

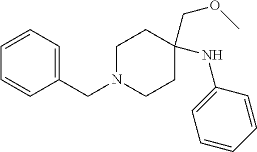 [SMILES: COCC1(Nc2ccccc2)CCN(Cc2ccccc2)CC1]

To a solution of (1-benzyl-4-(phenylamino)piperidin-4-yl)methanol (90 mg, 0.304 mmol) in N,N-dimethylformamide (2.5 mL) was added sodium hydride (24 mg, 60% in oil, 0.608 mmol) at 0° C., After stirring for 15 minutes, iodomethane (44 mg, 0.304 mmol) was added. The resulting mixture was stirred for 15 minutes. The reaction was quenched with ice-water (10 mL). The mixture was extracted with ethyl acetate (3×50 mL). The combined organic layers were dried over anhydrous sodium sulfate and concentrated under reduced pressure. The residue was purified by silica gel column chromatography eluting with ethyl acetate:petroleum ether=1:3 to afford 1-benzyl-4-(methoxymethyl)-N-phenylpiperidin-4-amine as a yellow oil (85 mg, 90%).

LCMS (ESI): m/z=311.2 [M+H]⁺.

### 4-(Methoxymethyl)-N-phenylpiperidin-4-amine

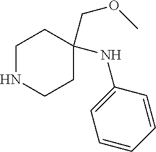 [SMILES: COCC1(Nc2ccccc2)CCNCC1]

To a solution of 1-benzyl-4-(methoxymethyl)-N-phenylpiperidin-4-amine (60 mg, 0.186 mmol) in acetic acid (3 mL) was added 5% palladium on carbon (60 mg, 5%). The resulting suspension was stirred under a hydrogen atmosphere for 3 hours. The catalyst was removed by a filtration and the acetic acid was removed under reduced pressure to afford 4-(methoxymethyl)-N-phenylpiperidin-4-amine as a yellow oil (45 mg, crude), which was used directly without further purification.

LCMS (ESI): m/z=221.2 [M+H]⁺.

### (R)-Methyl-3-methyl-2-(3-methylbenzamido)butanoate

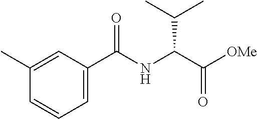 [SMILES: COC(=O)[C@H](NC(=O)c1cccc(C)c1)C(C)C]

To a suspension of (R)-methyl-2-amino-3-methylbutanoate hydrochloride (2.9 g, 17 mmol) in dichloromethane (30 mL) was added N,N-diisopropylethylamine (5.6 g, 40 mmol). To the resulting mixture was added dropwise a solution of 3-methylbenzoyl chloride (3.2 g, 20.6 mmol) in dichloromethane (5 mL) at 0° C. The mixture was stirred for 1 hour before the reaction was quenched with ice-water (10 mL). The organic layer was collected and the aqueous layer was extracted with ethyl acetate (3×20 mL). The combined organic layers were dried over anhydrous sodium sulfate and the solvent was removed under reduced pressure. The residue was purified by silica gel column chromatography eluting with ethyl acetate:petroleum ether=1:10 to afford (R)-methyl-3-methyl-2-(3-methylbenzamido)butanoate as a yellow oil (3.3 g, 78%).

LCMS (ESI): m/z=250.1 [M+H]⁺.

¹H-NMR (300 MHz, CDCl₃): δ=1.01 (t, J=7.2 Hz, 6H), 2.19-2.36 (m, 1H), 2.42 (s, 3H), 3.79 (s, 3H), 4.59-4.83 (m, 1H), 6.55-6.66 (m, 1H), 7.30-7.38 (m, 2H), 7.56-7.66 (m, 2H).

### (R)-3-Methyl-2-(3-methylbenzamido)butanoic acid

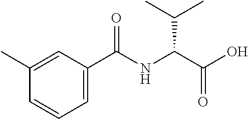 [SMILES: Cc1cccc(C(=O)N[C@@H](C(=O)O)C(C)C)c1]

To a solution of (R)-methyl-3-methyl-2-(3-methylbenzamido)butanoate (1.5 g, 6 mmol) in tetrahydrofuran (12 mL) was added an aqueous lithium hydroxide solution (8 mL, 2.0 M, 0.016 mol). Before quenching with ice-water (20 mL), the reaction was stirred for 1 hour. The pH of the solution was adjusted to 3 by addition of a 5% aqueous hydrochloric acid solution. The mixture was filtered and the filter cake was washed with petroleum ether (30 mL) and dried under reduced pressure to afford (R)-3-methyl-2-(3-methyl benzamido)butanoic acid as a thick oil (1.1 g, 77%).

LCMS (ESI): m/z=236.1 [M+H]⁺.

### Example 17

### (R)—N-(1-(4-(Methoxymethyl)-4-(phenylamino)piperidin-1-yl)-3-methyl-1-oxobutan-2-yl)-3-methylbenzamide

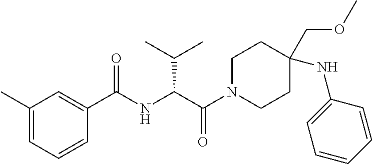 [SMILES: COCC1(Nc2ccccc2)CCN(C(=O)[C@H](NC(=O)c2cccc(C)c2)C(C)C)CC1]

To a suspension of 4-(methoxymethyl)-N-phenylpiperidin-4-amine (45 mg, 0.204 mmol) and (R)-3-methyl-2-(3-methylbenzamido)butanoic acid (63 mg, 0.265 mmol) in dichloromethane (5 mL) was added 1-[bis(dimethylamino)methylene]-1H-1,2,3-triazolo[4,5-b]pyridinium-3-oxide hexafluorophosphate (HATU) (108 mg, 0.306 mmol) and N,N-diisopropylethylamine (79 mg, 0.613 mmol). The resulting mixture was stirred for 2 hours at room temperature before the reaction was quenched with ice-water. The mixture was extracted with ethyl acetate (3×10 mL) and the combined organic layers were dried over anhydrous sodium sulfate and concentrated under reduced pressure. The residue was purified by prep-TLC with dichloromethane:methanol=15:1 to afford (R)—N-(1-(4-(methoxymethyl)-4-(phenylamino)piperidin-1-yl)-3-methyl-1-oxobutan-2-yl)-3-methylbenzamide (Example 17) as a white solid (13 mg, 13% over two steps).

LCMS (ESI): m/z=438.2 [M+H]⁺.

¹H-NMR (400 MHz, CD₃OD): (=1.04-1.88 (m, 15H), 2.40 (s, 3H), 3.09 (s, 3H), 3.46-3.47 (m, 1H), 3.92-4.51 (m, 3H), 4.83-4.85 (m, 1H), 7.15-7.52 (m, 9H).

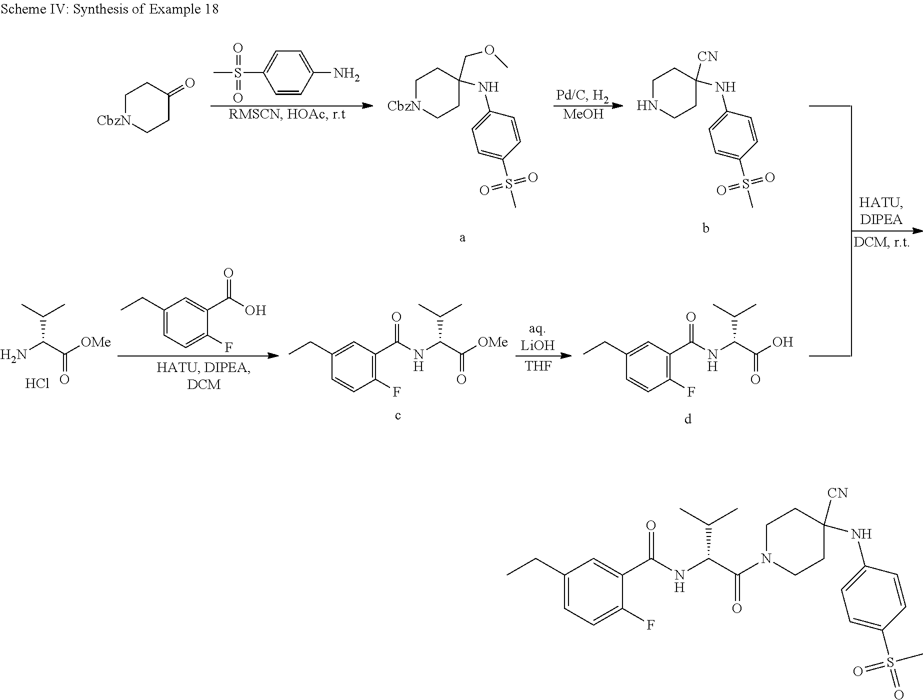

### Benzyl-4-cyano-4-(4-(methylsulfonyl)phenylamino)piperidine-1-carboxylate

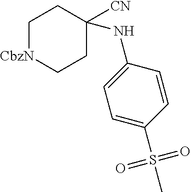 [SMILES: CS(=O)(=O)c1ccc(NC2(C#N)CCN(C(=O)OCc3ccccc3)CC2)cc1]

To a solution of 4-(methylsulfonyl)aniline (2.2 g, 0.013 mol) in acetic acid (20 mL) was added benzyl 4-oxopiperidine-1-carboxylate (3.3 g, 0.014 mol). The resulting mixture was cooled to 0° C. and trimethylsilyl cyanide (1.53 g, 0.014 mol) was added. The resulting solution was stirred overnight. Saturated aqueous ammonium chloride solution was added (50 mL). The mixture was filtered, the filter cake was washed with water (3×10 mL) and dried under reduced pressure to afford benzyl-4-cyano-4-(4-(methylsulfonyl)phenylamino)piperidine-1-carboxylate as a white solid (4.7 g, 91%).

LCMS (ESI): m/z=414.1 [M+H]⁺.

### 4-(4-(Methylsulfonyl)phenylamino)piperidine-4-carbonitrile

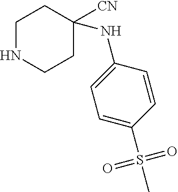 [SMILES: CS(=O)(=O)c1ccc(NC2(C#N)CCNCC2)cc1]

To a solution of benzyl-4-cyano-4-(4-(methylsulfonyl)phenylamino)piperidine-1-carboxylate (200 mg, 0.44 mmol) in methanol (20 mL) was added 5% palladium on carbon (15 mg, 5%). The mixture was stirred for 1 hour under a hydrogen atmosphere before the catalyst was removed by filtration. The filtrate was concentrated under reduced pressure to afford 4-(4-(methylsulfonyl)phenylamino)piperidine-4-carbonitrile (100 mg, 74%). LCMS (ESI): m/z=280.2 [M+H]⁺.

### (R)-Methyl-2-(5-ethyl-2-fluorobenzamido)-3-methylbutanoate

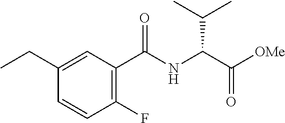 [SMILES: CCc1ccc(F)c(C(=O)N[C@@H](C(=O)OC)C(C)C)c1]

To a solution of 5-ethyl-2-fluorobenzoic acid (1.00 g, 5.95 mmol) in dichloromethane (50 mL) was added sequentially D-valine methyl ester hydrochloride (1.2 g, 7.18 mmol), 1-[bis(dimethylamino)methylene]-1H-1,2,3-triazolo[4,5-b]pyridinium-3-oxide hexafluorophosphate (HATU) (3.4 g, 8.94 mmol) and N,N-diisopropylethylamine (1.9 g, 14.7 mmol). Before quenching with ice-water (50 mL), the reaction was stirred for 2 hours. The mixture was extracted with dichloromethane (3×100 mL). The combined organic layers were dried over anhydrous sodium sulfate and the solvent was removed under reduced pressure. The residue was purified by silica gel column chromatography eluting with ethyl acetate:petroleum ether=1:4 to afford (R)-methyl-2-(5-ethyl-2-fluorobenzamido)-3-methylbutanoate as a thick oil (1.6 g, 95%).

LCMS (ESI): m/z=282.2 [M+H]⁺.

### (R)-2-(5-Ethyl-2-fluorobenzamido)-3-methylbutanoic acid

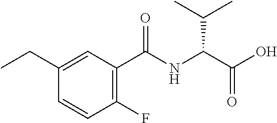 [SMILES: CCc1ccc(F)c(C(=O)N[C@@H](C(=O)O)C(C)C)c1]

To a solution of (R)-methyl-2-(5-ethyl-2-fluorobenzamido)-3-methylbutanoate (1.6 g, crude) in tetrahydrofuran (20 mL) was added an aqueous lithium hydroxide solution (240 mg in 10 mL of water, 17.8 mmol). Before quenching with ice-water (20 mL), the reaction was stirred for 1 hour. The pH of the solution was adjusted to 3 by addition of a 9% aqueous hydrochloric acid solution. The mixture was filtered and the filter cake was washed with petroleum ether (30 mL) and dried under reduced pressure to afford (R)-2-(5-ethyl-2-fluorobenzamido)-3-methylbutanoic acid as a white solid (1.3 g, 94% over two steps).

LCMS (ESI): m/z=268.1 [M+H]⁺.

¹H-NMR (300 MHz, CDCl₃): δ=1.05 (dd, J=8.6, 6.9 Hz, 6H), 1.23 (t, J=7.6 Hz, 3H), 2.31-2.45 (m, 1H), 2.65 (q, J=7.8 Hz, 2H), 7.01-7.07 (m, 1H), 7.16-7.33 (m, 2H), 7.85-7.93 (m, 1H).

### Example 18

### (R)—N-(1-(4-Cyano-4-(4-(methylsulfonyl)phenylamino)piperidin-1-yl)-3-methyl-1-oxobutan-2-yl)-5-ethyl-2-fluorobenzamide

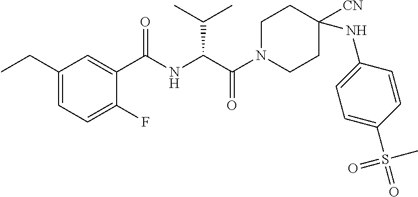 [SMILES: CCc1ccc(F)c(C(=O)N[C@@H](C(=O)N2CCC(C#N)(Nc3ccc(S(C)(=O)=O)cc3)CC2)C(C)C)c1]

To a solution of 4-(4-(methylsulfonyl)phenylamino)piperidine-4-carbonitrile hydrochloride (100 mg, 0.35 mmol) in dichloromethane (10 mL) was added sequentially (R)-2-(5-ethyl-2-fluorobenzamido)-3-methylbutanoic acid (109 mg, 0.40 mmol), 1-[bis(dimethylamino)methylene]-1H-1,2,3-triazolo[4,5-b]pyridinium-3-oxide hexafluorophosphate (HATU) (204 mg, 0.54 mmol), and N,N-diisopropylethylamine (138 mg, 1.075 mmol). The resulting mixture was stirred for 2 hours at room temperature before the reaction was quenched with ice-water (10 mL). The mixture was extracted with dichloromethane (3×10 mL) and the combined organic layers were dried over anhydrous sodium sulfate and concentrated under reduced pressure. The residue was purified by prep-TLC with dichloromethane: methanol=15:1 to afford (R)—N-(1-(4-cyano-4-(4-(methylsulfonyl)phenylamino)piperidin-1-yl)-3-methyl-1-oxobutan-2-yl)-5-ethyl-2-fluorobenzamide (Example 18) as a white solid (23.6 mg, 12%).

LCMS (ESI): m/z=529.2 [M+H]⁺.

¹H-NMR (400 MHz, CD₃OD): δ=1.02-1.07 (m, 6H), 1.21-1.28 (m, 3H), 1.89-2.14 (m, 3H), 2.45-2.49 (m, 2H), 2.60-2.66 (m, 2H), 3.08 (s, 3H), 3.27-3.29 (m, 1H), 3.63-3.65 (m, 1H), 4.02-4.29 (m, 2H), 4.87-4.97 (m, 1H), 7.13-8.28 (m, 8H).

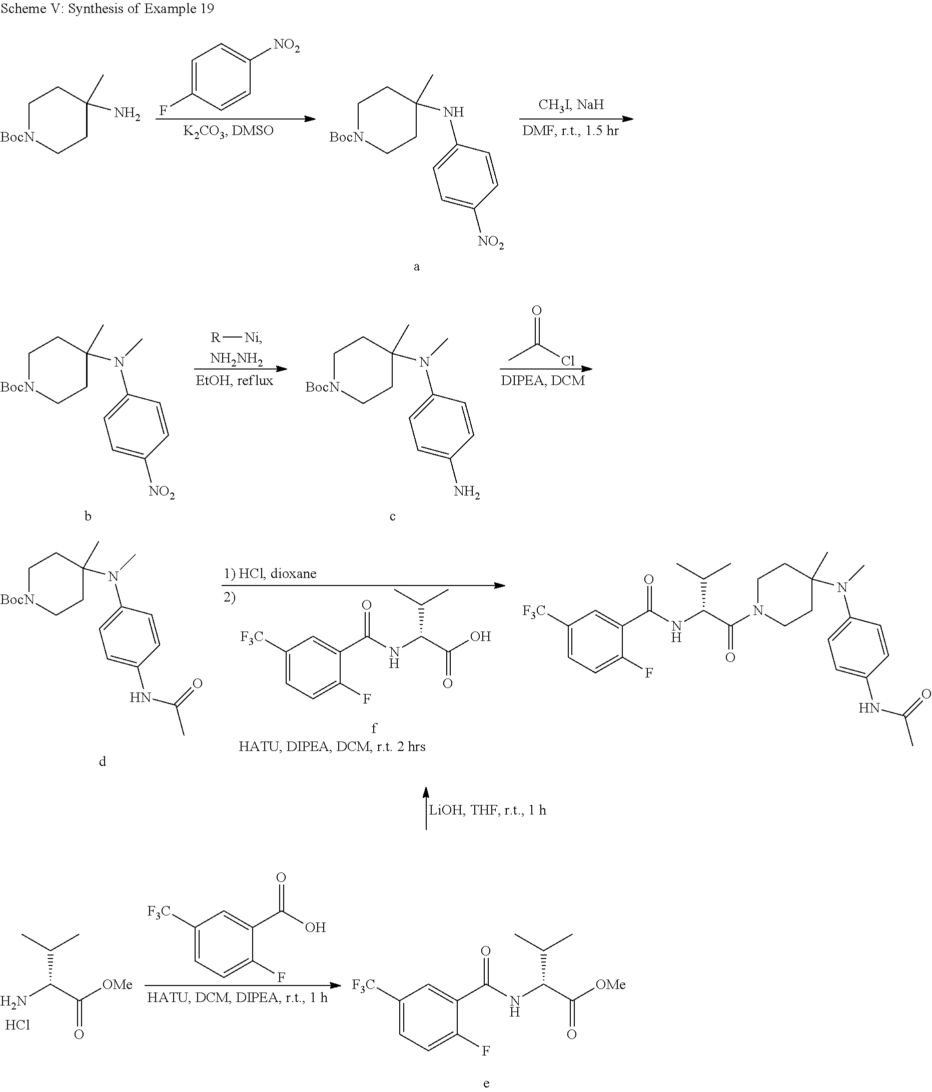

### tert-Butyl-4-methyl-4-(4-nitrophenylamino)piperidine-1-carboxylate

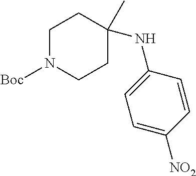 [SMILES: CC1(Nc2ccc([N+](=O)[O-])cc2)CCN(C(=O)OC(C)(C)C)CC1]

To a solution of tert-butyl-4-amino-4-methylpiperidine-1-carboxylate (50 mg, 0.23 mmol) in dimethyl sulfoxide (2 mL) was added 1-fluoro-4-nitrobenzene (39.5 mg, 0.28 mmol) and potassium carbonate (50 mg, 0.36 mmol). The resulting mixture was heated at 120° C. for 60 hours. The reaction was quenched with ice-water (10 mL). The mixture was extracted with ethyl acetate (3×10 mL). The combined organic layers were dried over anhydrous sodium sulfate and concentrated under reduced pressure. The residue was purified by prep-TLC (ethyl acetate:petroleum ether=1:2) to afford tert-butyl-4-methyl-4-(4-nitrophenylamino)piperidine-1-carboxylate as a yellow oil (30 mg, 38%).

LCMS (ESI): m/z=336.1 [M+H]⁺.

### tert-Butyl-4-methyl-4-(methyl(4-nitrophenyl)amino)piperidine-1-carboxylate

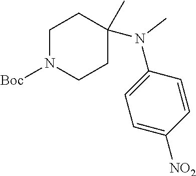 [SMILES: CN(c1ccc([N+](=O)[O-])cc1)C1(C)CCN(C(=O)OC(C)(C)C)CC1]

To a solution of tert-butyl-4-methyl-4-(4-nitrophenylamino)piperidine-1-carboxylate (30 mg, 0.09 mmol) in N,N-dimethylformamide (2 mL) was added sodium hydride (50 mg, 60% in oil, 0.13 mmol) at 0° C., After stirring for 15 minutes, iodomethane (25 mg, 0.18 mmol) was added. The resulting mixture was stirred for 15 minutes at room temperature and the reaction was quenched with ice-water (10 mL). The mixture was extracted with ethyl acetate (3×50 mL). The combined organic layers were dried over anhydrous sodium sulfate and the solvent was removed under reduced pressure to afford tert-butyl-4-methyl-4-(methyl(4-nitrophenyl)amino)piperidine-1-carboxylate as a yellow oil (30 mg, crude), which was used directly without further purification.

LCMS (ESI): m/z=350.2 [M+H]⁺.

### tert-Butyl-4-((4-aminophenyl)(methyl)amino)-4-methylpiperidine-1-carboxylate

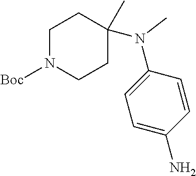 [SMILES: CN(c1ccc(N)cc1)C1(C)CCN(C(=O)OC(C)(C)C)CC1]

To a solution of tert-butyl-4-methyl-4-(methyl(4-nitrophenyl)amino)piperidine-1-carboxylate (30 mg, crude) in ethanol (5 mL) was added Raney-Ni (50 mg, 50% in ethanol). After heating to 50° C., hydrazine (50 mg, 1 mmol) was added dropwise to the suspension. Before removing the Raney-Ni by filtration, the reaction mixture was stirred at 50° C. for 30 minutes. The solvent was removed under reduced pressure and the residue was used directly without further purification.

LCMS (ESI): m/z=320.2 [M+H]⁺.

### tert-Butyl-4-((4-acetamidophenyl)(methyl)amino)-4-methylpiperidine-1-carboxylate

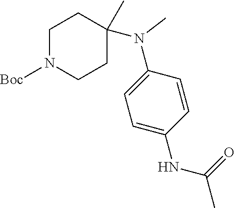 [SMILES: CC(=O)Nc1ccc(N(C)C2(C)CCN(C(=O)OC(C)(C)C)CC2)cc1]

To a solution of tert-butyl 4-((4-aminophenyl)(methyl)amino)-4-methylpiperidine-1-carboxylate (30 mg, crude) in dichloromethane (5 mL) was added N,N-diisopropylethylamine (24 mg, 0.19 mmol) at room temperature. After stirring for 10 minutes, to the resulting mixture was added acetyl chloride (11 mg, 0.14 mmol). Before quenching by addition of 12 mL ice-water, the reaction was stirred for 1.5 hours at room temperature. The mixture was extracted with ethyl acetate (3×100 mL). The combined organic layers were dried over anhydrous sodium sulfate and concentrated under reduced pressure. The residue was purified by prep-TLC (ethyl acetate:petroleum ether=1:1) to afford tert-butyl-4-((4-acetamidophenyl)(methyl)amino)-4-methylpiperidine-1-carboxylate as a colorless oil (24 mg, 75% over 3 steps).

LCMS (ESI): m/z=362.4 [M+H]⁺.

### (R)-Methyl-2-(2-fluoro-5-(trifluoromethyl)benzamido)-3-methylbutanoate

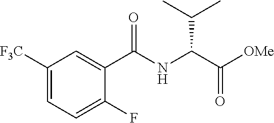 [SMILES: COC(=O)[C@H](NC(=O)c1cc(C(F)(F)F)ccc1F)C(C)C]

To a solution of 2-fluoro-5-(trifluoromethyl)benzoic acid (15.0 g, 0.072 mol) in dichloromethane (500 mL) was added sequentially D-valine methyl ester hydrochloride (12.0 g, 0.072 mol), 1-[bis(dimethylamino)methylene]-1H-1,2,3-triazolo[4,5-b]pyridinium-3-oxide hexafluorophosphate (HATU) (41 g, 0.11 mol) and N,N-diisopropylethylamine (23.3 g, 0.18 mol). Before quenching with ice-water (50 mL), the reaction was stirred for 1 hour. The mixture was extracted with dichloromethane (3×100 mL). The combined organic layers were dried over anhydrous sodium sulfate and the solvent was removed under reduced pressure. The residue was purified by silica gel column chromatography eluting with ethyl acetate:petroleum ether=1:5 to afford (R)-methyl-2-(2-fluoro-5-(trifluoromethyl)benzamido)-3-methylbutanoate as a thick oil (18.6 g, 81%).

LCMS (ESI): m/z=322.1 [M+H]⁺.

¹H-NMR (300 MHz, CD₃OD): δ=1.05 (dd, J=6.8, 4.3 Hz, 6H), 2.22-2.35 (m, 1H), 3.79 (s, 3H), 4.58 (d, J=6.0 Hz, 1H), 7.46 (t, J=9.4 Hz, 1H), 7.89 (m, 1H), 8.00 (dd, J=6.2, 2.1 Hz, 1H).

### (R)-2-(2-Fluoro-5-(trifluoromethyl)benzamido)-3-methylbutanoic acid

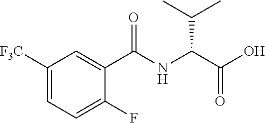 [SMILES: CC(C)[C@@H](NC(=O)c1cc(C(F)(F)F)ccc1F)C(=O)O]

To a solution of (R)-methyl-2-(2-fluoro-5-(trifluoromethyl)benzamido)-3-methylbutanoate (18.6 g, 0.058 mol) in tetrahydrofuran (50 mL) was added an aqueous lithium hydroxide solution (44 mL, 2.0 M, 0.088 mol). Before quenching with ice-water (20 mL), the reaction was stirred 1 hour. The pH of the solution was adjusted to 3 by addition of a 5% aqueous hydrochloric acid solution (5%). The mixture was filtered and the filter cake was washed with petroleum ether (30 mL) and dried under reduced pressure to afford (R)-2-(2-fluoro-5-(trifluoromethyl)benzamido)-3-methylbutanoic acid as a white solid (16.7 g, 94%).

LCMS (ESI): m/z=308.1 [M+H]⁺.

¹H-NMR (300 MHz, DMSO): δ=0.95 (dd, J=6.7, 4.7 Hz, 6H), 2.13-2.17 (m, 1H), 4.31-4.35 (m, 1H), 7.52-7.55 (m, 1H), 7.58-7.85 (m, 2H), 12.77 (br, 1H).

### Example 19

### (R)—N-(1-(4-(4-Acetamidophenyl(methyl)amino)-4-methylpiperidin-1-yl)-3-methyl-1-oxobutan-2-yl)-2-fluoro-5-(trifluoromethyl)benzamide

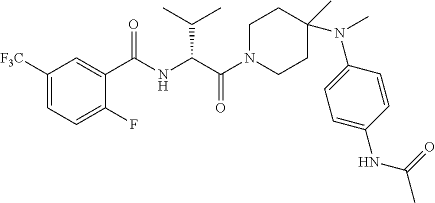 [SMILES: CC(=O)Nc1ccc(N(C)C2(C)CCN(C(=O)[C@H](NC(=O)c3cc(C(F)(F)F)ccc3F)C(C)C)CC2)cc1]

A solution of tert-butyl-4-((4-acetamidophenyl)(methyl)amino)-4-methylpiperidine-1-carboxylate (24 mg, 0.066 mmol) in a 6.0 M solution of hydrochloric acid in dioxane (10 mL) was stirred for 30 minutes. The solvent was removed under reduced pressure. To the residue was added dichloromethane (5 mL), (R)-2-(2-fluoro-5-(trifluoromethyl)benzamido)-3-methylbutanoic acid (22 mg, 0.07 mmol), 1-[bis(dimethylamino)methylene]-1H-1,2,3-triazolo[4,5-b]pyridinium-3-oxide hexafluorophosphate (HATU) (38 mg, 0.10 mmol) and N,N-diisopropylethylamine (21 mg, 0.17 mmol). The resulting mixture was stirred for 2 hours at room temperature before the reaction was quenched with ice-water (10 mL). The mixture was extracted with ethyl acetate (3×10 mL) and the combined organic layers were dried over anhydrous sodium sulfate and concentrated under reduced pressure. The residue was purified by prep-TLC with dichloromethane: methanol=15:1 to afford (R)—N-(1-(4-(4-acetamidophenyl(methyl)amino)-4-methylpiperidin-1-yl)-3-methyl-1-oxobutan-2-yl)-2-fluoro-5-(trifluoromethyl)benzamide (Example 19) as a white solid (6.2 mg, 17%).

LCMS (ESI): m/z=551.2 [M+H]⁺.

¹H-NMR (400 MHz, CD₃OD): δ=1.01-1.08 (m, 6H), 1.59 (s, 3H), 1.82-2.01 (m, 3H), 2.13-2.22 (m, 5H), 2.83-2.89 (m, 3H), 3.38-3.44 (m, 1H), 3.64-3.82 (m, 1H), 4.21-4.75 (m, 2H), 4.75-4.80 (m, 1H), 7.37-7.56 (m, 3H), 7.80-8.05 (m, 4H).

### Example 20

### Prepared Using a Method Analogous to Example 19

### (R)-3-Methyl-N-(3-methyl-1-(4-methyl-4-(methyl(phenyl)amino)piperidin-1-yl)-1-oxobutan-2-yl)benzamide

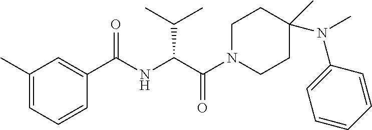 [SMILES: Cc1cccc(C(=O)N[C@@H](C(=O)N2CCC(C)(N(C)c3ccccc3)CC2)C(C)C)c1]

23.8 mg, yield: 44%, appearance: white solid.

LCMS (ESI): m/z=422.2 [M+H]⁺.

¹H-NMR (400 MHz, CD₃OD): δ=0.90-1.05 (m, 9H), 1.45-2.11 (m, 5H), 2.39 (s, 3H), 2.76 (s, 3H), 3.58-3.81 (m, 4H), 5.09-5.12 (m, 1H), 7.05-7.65 (m, 8H).

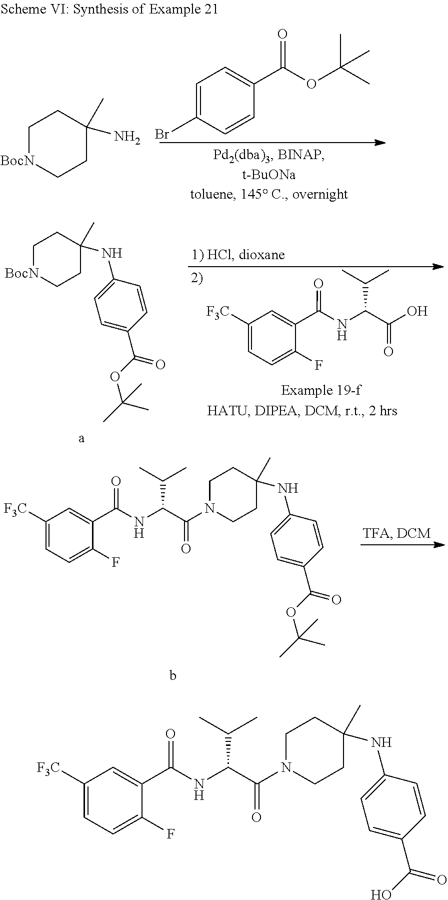 [SMILES: CC1(N)CCN(C(=O)OC(C)(C)C)CC1 ; CC(C)(C)OC(=O)c1ccc(Br)cc1 ; CC1(Nc2ccc(C(=O)OC(C)(C)C)cc2)CCN(C(=O)OC(C)(C)C)CC1 ; CC(C)[C@@H](NC(=O)c1cc(C(F)(F)F)ccc1F)C(=O)O ; CC(C)[C@@H](NC(=O)c1cc(C(F)(F)F)ccc1F)C(=O)N1CCC(C)(Nc2ccc(C(=O)OC(C)(C)C)cc2)CC1 ; CC(C)[C@@H](NC(=O)c1cc(C(F)(F)F)ccc1F)C(=O)N1CCC(C)(Nc2ccc(C(=O)O)cc2)CC1]

### tert-Butyl-4-(4-(tert-butoxycarbonyl)phenylamino)-4-methylpiperidine-1-carboxylate

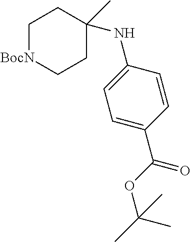 [SMILES: CC1(Nc2ccc(C(=O)OC(C)(C)C)cc2)CCN(C(=O)OC(C)(C)C)CC1]

To a solution of tert-butyl-4-amino-4-methylpiperidine-1-carboxylate (90 mg, 0.35 mmol) in xylene (10 mL) was added sequentially tert-butyl-4-bromobenzoate (50 mg, 0.23 mmol), tris(dibenzylideneacetone)dipalladium(0) (Pd₂(dba)₃) (16 mg, 0.03 mmol), 2,2′-bis(diphenylphosphino)-1,1′-binaphthyl (BINAP) (38 mg, 0.06 mmol) and sodium tert-butoxide (45 mg, 0.47 mmol) under an argon atmosphere. The reaction mixture was heated to 145° C. overnight. The solvent was removed under reduced pressure. The residue was purified by prep-TLC (ethyl acetate:petroleum ether=1:5) to give tert-butyl-4-(4-(tert-butoxycarbonyl)phenylamino)-4-methylpiperidine-1-carboxylate as a brown oil (30 mg, crude), which was used directly without further purification.

LCMS (ESI): m/z=391.2 [M+H]⁺.

### (R)-tert-Butyl-4-(1-(2-(2-fluoro-5-(trifluoromethyl)benzamido)-3-methylbutanoyl)-4-methyl-piperidin-4-ylamino)benzoate

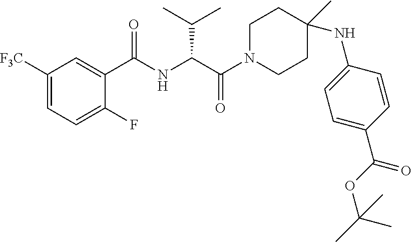 [SMILES: CC(C)[C@@H](NC(=O)c1cc(C(F)(F)F)ccc1F)C(=O)N1CCC(C)(Nc2ccc(C(=O)OC(C)(C)C)cc2)CC1]

A solution of tert-butyl-4-(4-(tert-butoxycarbonyl)phenylamino)-4-methylpiperidine-1-carboxylate (40 mg, 0.1 mmol) in a 6.0 M solution of hydrochloric acid in dioxane (10 mL) was stirred for 30 minutes. The solvent was removed under reduced pressure. To the residue was added dichloromethane (5 mL), (R)-2-(2-fluoro-5-(trifluoromethyl)benzamido)-3-methylbutanoic acid (prepared as described in Example 19-f) (39 mg, 0.1 mmol), 1-[bis(dimethylamino)methylene]-1H-1,2,3-triazolo[4,5-b]pyridinium-3-oxide hexafluorophosphate (HATU) (58 mg, 0.15 mmol) and N,N-diisopropylethylamine (33 mg, 0.25 mmol). The reaction was stirred for 2 hours at room temperature before quenching with ice-water (10 mL). The mixture was extracted with ethyl acetate (3×10 mL) and the combined organic layers were dried over anhydrous sodium sulfate and concentrated under reduced pressure. The residue was purified by prep-HPLC (acetonitrile/water, acetonitrile from 30% to 60%) to afford (R)-tert-butyl-4-(1-(2-(2-fluoro-5-(trifluoromethyl)benzamido)-3-methylbutanoyl)-4-methylpiperidin-4-ylamino)benzoate as a colorless oil (14 mg, 24% over two steps).

LCMS (ESI): m/z=580.2 [M+H]⁺.

### Example 21

### (R)-4-(1-(2-(2-Fluoro-5-(trifluoromethyl)benzamido)-3-methylbutanoyl)-4-methylpiperidin-4-ylamino)benzoic acid

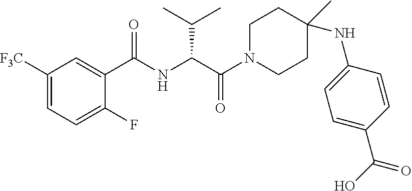 [SMILES: CC(C)[C@@H](NC(=O)c1cc(C(F)(F)F)ccc1F)C(=O)N1CCC(C)(Nc2ccc(C(=O)O)cc2)CC1]

To a solution of (R)-tert-butyl-4-(1-(2-(2-fluoro-5-(trifluoromethyl)benzamido)-3-methylbutanoyl)-4-methylpiperidin-4-ylamino)benzoate (14 mg, 0.024 mmol) in dichloromethane (2 mL) was added trifluoroacetic acid (2 mL). The reaction was stirred 2 hours. The solvent was removed under reduced pressure. The residue was purified by prep-HPLC to afford (R)-4-(1-(2-(2-fluoro-5-(trifluoromethyl)benzamido)-3-methylbutanoyl)-4-methylpiperidin-4-ylamino)benzoic acid (Example 21) as a white solid (12.8 mg, 100%).

LCMS (ESI): m/z=524.2 [M+H]⁺.

¹H-NMR (400 MHz, CD₃OD): δ=1.01-1.07 (m, 6H), 1.14-1.50 (m, 3H), 1.66-1.88 (m, 2H), 2.05-2.28 (m, 3H), 3.40-4.03 (m, 4H), 4.94-4.98 (m, 1H), 6.90-6.93 (m, 1H), 7.02-7.04 (m, 1H), 7.44-7.50 (m, 1H), 7.85-7.93 (m, 3H), 8.03-8.06 (m, 1H).

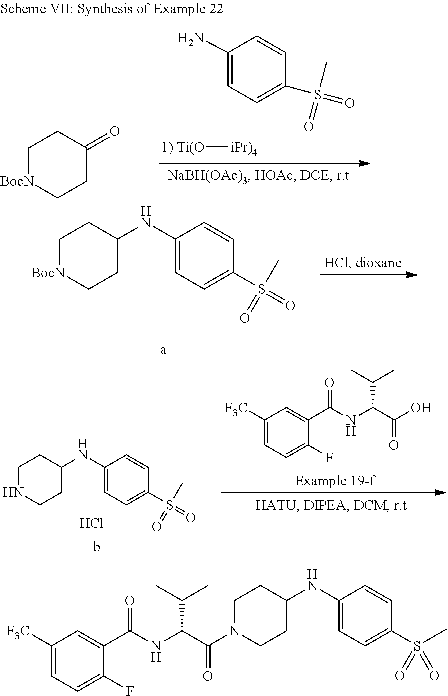

### tert-Butyl-4-(4-(methylsulfonyl)phenylamino)piperidi ne-1-carboxylate

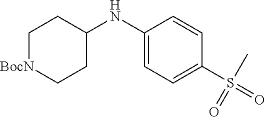 [SMILES: CC(C)(C)OC(=O)N1CCC(Nc2ccc(S(C)(=O)=O)cc2)CC1]

To a solution of tert-butyl-4-oxopiperidine-1-carboxylate (1.0 g, 5.0 mmol) and 4-(methylsulfonyl)aniline (1.03 g, 6.0 mmol) in dichloromethane (30 mL) was added acetic acid (0.3 g, 5 mmol). After stirring for 15 minutes, sodium triacetoxyborohydride (3.2 g, 15 mmol) was added to the solution. The resulting mixture was stirred overnight and the reaction was quenched with a 1.0 M aqueous sodium hydroxide solution (10 mL). The mixture was extracted with ethyl acetate (3×50 mL). The combined organic layers were dried over anhydrous sodium sulfate and concentrated under reduced pressure. The residue was purified by silica gel column chromatography eluting with ethyl acetate:petroleum ether=1:3 to afford tert-butyl-4-(4-(methylsulfonyl)phenylamino)piperidine-1-carboxylate as a yellow oil (0.2 g, 11%).

LCMS (ESI): m/z=355.1 [M+H]⁺.

### N-(4-(Methylsulfonyl)phenyl)piperidin-4-amine hydrochloride

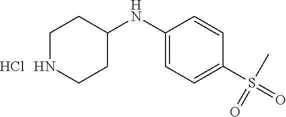 [SMILES: Cl ; CS(=O)(=O)c1ccc(NC2CCNCC2)cc1]

A solution of tert-butyl-4-(4-(methylsulfonyl)phenylamino)piperidine-1-carboxylate (190 mg, 0.56 mmol) in a 6.0 M solution of hydrochloric acid in dioxane (10 mL) was stirred for 30 minutes. A white solid formed which was collected by filtration. The solid was dried under reduced pressure to afford N-(4-(methylsulfonyl)phenyl)piperidin-4-amine hydrochloride (170 mg, some residual solvent).

LCMS (ESI): m/z=255.1 [M+H]⁺.

### Example 22

### (R)-2-Fluoro-N-(3-methyl-1-(4-(4-(methylsulfonyl)phenylamino)piperidin-1-yl)-1-oxobutan-2-yl)-5-(trifluoromethyl)benzamide

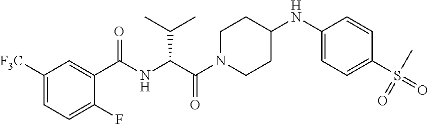 [SMILES: CC(C)[C@@H](NC(=O)c1cc(C(F)(F)F)ccc1F)C(=O)N1CCC(Nc2ccc(S(C)(=O)=O)cc2)CC1]

To a suspension of N-(4-(methylsulfonyl)phenyl)piperidin-4-amine hydrochloride (106 mg, 0.34 mmol) and (R)-2-(2-fluoro-5-(trifluoromethyl)benzamido)-3-methylbutanoic acid (prepared as described in Example 19-f) (100 mg, 0.34 mmol) in dichloromethane (5 mL) was added 1-[bis(dimethylamino)methylene]-1H-1,2,3-triazolo[4,5-b]pyridinium-3-oxide hexafluorophosphate (HATU) (260 mg, 0.68 mmol) and N,N-diisopropylethylamine (180 mg, 1.36 mmol). The resulting mixture was stirred for 2 hours at room temperature before the reaction was quenched with ice-water (10 ml). The mixture was extracted with ethyl acetate (3×10 mL) and the combined organic layers were dried over anhydrous sodium sulfate and concentrated under reduced pressure. The residue was purified by prep-TLC with dichloromethane:methanol=15:1 to afford (R)-2-fluoro-N-(3-methyl-1-(4-(4-(methylsulfonyl)phenylamino)piperidin-1-yl)-1-oxobutan-2-yl)-5-(trifluoromethyl)benzamide (Example 22) as a white solid (50 mg, 25%).

LCMS (ESI): m/z=544.1 [M+H]⁺.

¹H-NMR (400 MHz, CD₃OD): δ=1.04-1.07 (m, 6H), 1.30-1.51 (m, 2H), 2.06-2.18 (m, 3H), 2.98-3.12 (m, 4H), 3.21 (m, 1H), 3.21-3.31 (m, 1H), 3.71 (s, 1H), 4.37-4.11 (m, 2H), 5.00-5.01 (m, 1H), 6.75-6.78 (m, 2H), 7.43-7.46 (m, 2H), 7.48-7.62 (m, 2H), 7.85 (s, 1H), 8.05-8.07 (m, 1H).

### Example 23

### Prepared Using a Method Analogous to Example 22

### (R)—N-(1-(4-(1H-Indazol-5-ylamino)piperidin-1-yl)-3-methyl-1-oxobutan-2-yl)-2-fluoro-5-(trifluoromethyl)benzamide

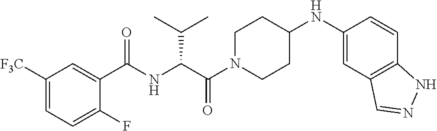 [SMILES: CC(C)[C@@H](NC(=O)c1cc(C(F)(F)F)ccc1F)C(=O)N1CCC(Nc2ccc3[nH]ncc3c2)CC1]

10.3 mg, yield: 6%, appearance: white solid.

LCMS (ESI): m/z=506.2 [M+H]⁺.

¹H-NMR (400 MHz, CD₃OD): δ=1.00-1.09 (m, 6H), 1.63-1.75 (m, 1H), 1.88-1.94 (m, 1H), 2.11-2.18 (m, 3H), 2.74-2.83 (m, 3H), 3.88-3.94 (m, 1H), 4.42-4.50 (m, 1H), 4.71-4.74 (m, 1H), 7.46-7.51 (m, 2H), 7.76-7.96 (m, 3H), 8.23 (s, 1H).

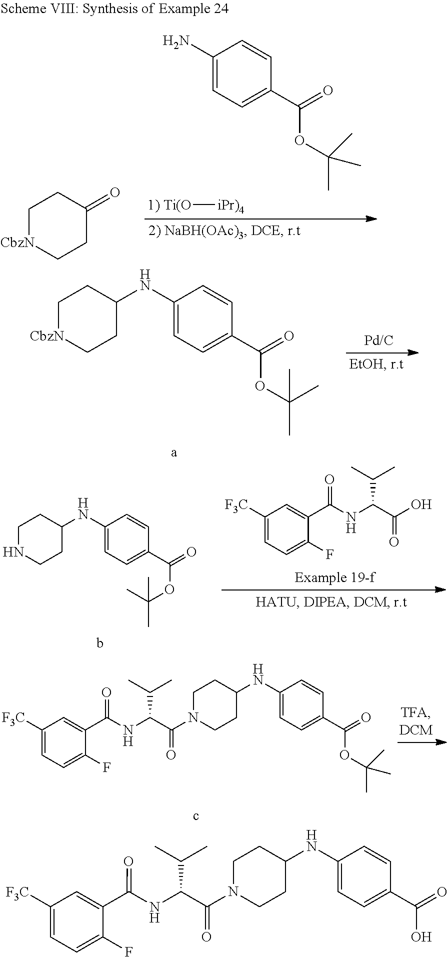

### Benzyl-4-(4-(tert-butoxycarbonyl)phenylamino)piperidine-1-carboxylate

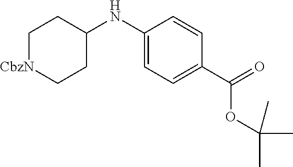 [SMILES: CC(C)(C)OC(=O)c1ccc(NC2CCN(C(=O)OCc3ccccc3)CC2)cc1]

To a solution of benzyl-4-oxopiperidine-1-carboxylate (1.0 g, 5.1 mmol) and tert-butyl 4-aminobenzoate (1.0 g, 4.2 mmol) and in dichloromethane (20 mL) was added acetic acid (0.26 g, 4.3 mmol). After stirring for 15 minutes, sodium triacetoxyborohydride (2.7 g, 12.7 mmol) was added to the solution. The resulting mixture was stirred overnight and the reaction was quenched with a 1.0 M aqueous sodium hydroxide solution (10 mL). The mixture was extracted with ethyl acetate (3×50 mL). The combined organic layers were dried over anhydrous sodium sulfate and concentrated under reduced pressure. The residue was purified by silica gel column chromatography eluting with ethyl acetate:petroleum ether=1:5 to afford benzyl 4-(4-(tert-butoxycarbonyl)phenylamino)piperidine-1-carboxylate as a white solid (0.39 g, 23%).

LCMS (ESI): m/z=411.2 [M+H]⁺.

### tert-Butyl-4-(piperidin-4-ylamino)benzoate

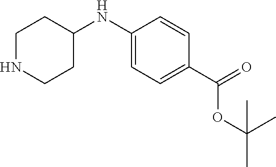 [SMILES: CC(C)(C)OC(=O)c1ccc(NC2CCNCC2)cc1]

To a solution of 4-(4-(tert-butoxycarbonyl)phenylamino)piperidine-1-carboxylate (330 mg, 0.8 mmol) was added 5% palladium on carbon (30 mg, 50% water). The suspension was stirred under a hydrogen atmosphere for 30 minutes and the catalyst was removed by filtration. The solvent was removed under reduced pressure to afford tert-butyl-4-(piperidin-4-ylamino)benzoate as a white solid (195 mg, 88%).

LCMS (ESI): m/z=277.2 [M+H]⁺.

### (R)-tert-Butyl-4-(1-(2-(2-fluoro-5-(trifluoromethyl)benzamido)-3-methylbutanoyl)piperidin-4-ylamino)benzoate

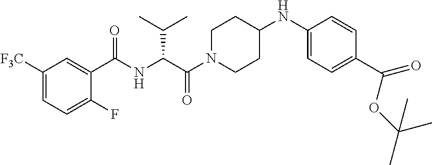 [SMILES: CC(C)[C@@H](NC(=O)c1cc(C(F)(F)F)ccc1F)C(=O)N1CCC(Nc2ccc(C(=O)OC(C)(C)C)cc2)CC1]

To a solution of tert-butyl-4-(piperidin-4-ylamino)benzoate (60 mg, 0.21 mmol) and (R)-2-(2-fluoro-5-(trifluoromethyl)benzamido)-3-methylbutanoic acid (prepared as described in Example 19-f) (60 mg, 0.19 mmol) in dichloromethane (5 mL) was added 1-[bis(dimethylamino)methylene]-1H-1,2,3-triazolo[4,5-b]pyridinium-3-oxide hexafluorophosphate (HATU) (160 mg, 0.42 mmol) and N,N-diisopropylethylamine (50 mg, 0.39 mmol). The resulting mixture was stirred for 2 hours at room temperature before quenching with ice-water (10 ml). The mixture was extracted with ethyl acetate (3×10 mL) and the combined organic layers were dried over anhydrous sodium sulfate and concentrated under reduced pressure. The residue was purified by prep-TLC with dichloromethane:methanol=15:1 to afford (R)-tert-butyl-4-(1-(2-(2-fluoro-5-(trifluoromethyl)benzamido)-3-methylbutanoyl)piperidin-4-ylamino)benzoate as a white solid (114 mg, 95%).

LCMS (ESI): m/z=566.2 [M+H]⁺.

### Example 24

### (R)-4-(1-(2-(2-Fluoro-5-(trifluoromethyl)benzamido)-3-methylbutanoyl)piperidin-4-ylamino)benzoic acid

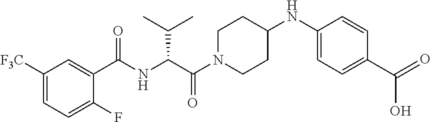 [SMILES: CC(C)[C@@H](NC(=O)c1cc(C(F)(F)F)ccc1F)C(=O)N1CCC(Nc2ccc(C(=O)O)cc2)CC1]

To a solution of (R)-tert-butyl-4-(1-(2-(2-fluoro-5-(trifluoromethyl)benzamido)-3-methylbutanoyl)piperidin-4-ylamino)benzoate (100 mg, 0.17 mmol) in dichloromethane was added trifluoroacetic acid (5 mL). The mixture was stirred for 2 hours. Dichloromethane and trifluoroacetic acid were removed under reduced pressure. The residue was purified by prep-HPLC (acetonitrile/water, acetonitrile from 30% to 60%) to afford (R)-4-(1-(2-(2-fluoro-5-(trifluoromethyl)benzamido)-3-methylbutanoyl)piperidin-4-ylamino)benzoic acid (Example 24) as a white solid (50 mg, 55%).

LCMS (ESI): m/z=510.1 [M+H]⁺.

¹H-NMR (400 MHz, CD₃OD): δ=1.74-1.78 (m, 6H), 2.04-2.62 (m, 2H), 2.72-2.89 (m, 3H), 3.32 (s, 3H), 3.68-3.72 (m, 1H), 4.08-4.12 (m, 1H), 4.42-4.45 (m, 1H), 4.93 (m, 1H), 5.12 (m, 1H), 5.63-5.67 (m, 1H), 7.43-7.46 (m, 1H), 8.31-8.48 (m, 1H), 8.48-8.63 (m, 2H), 8.69-8.74 (m, 2H), 9.52-9.54 (m, 1H).

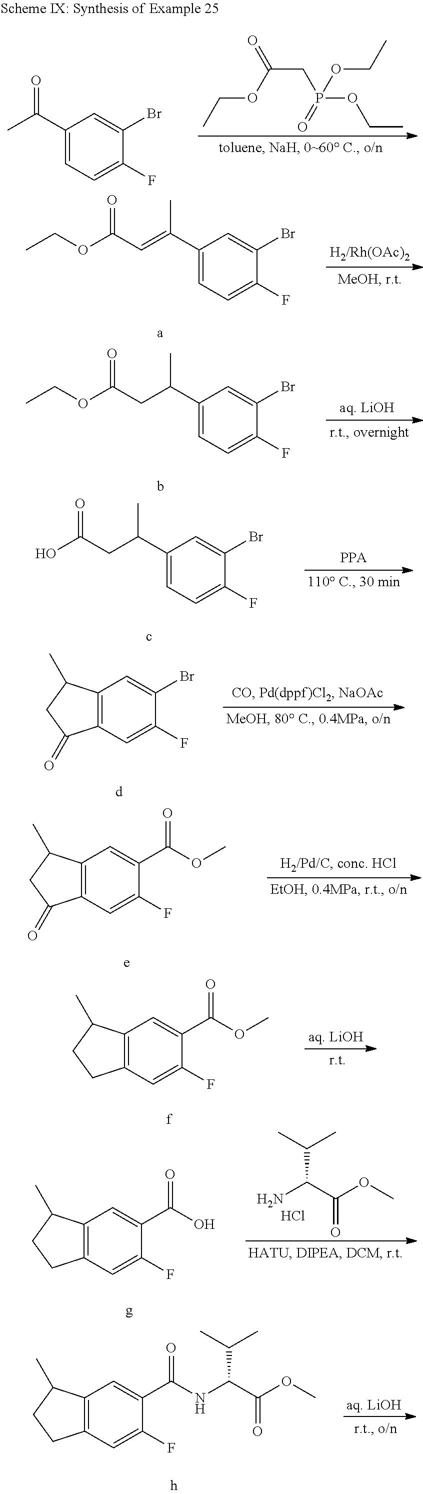

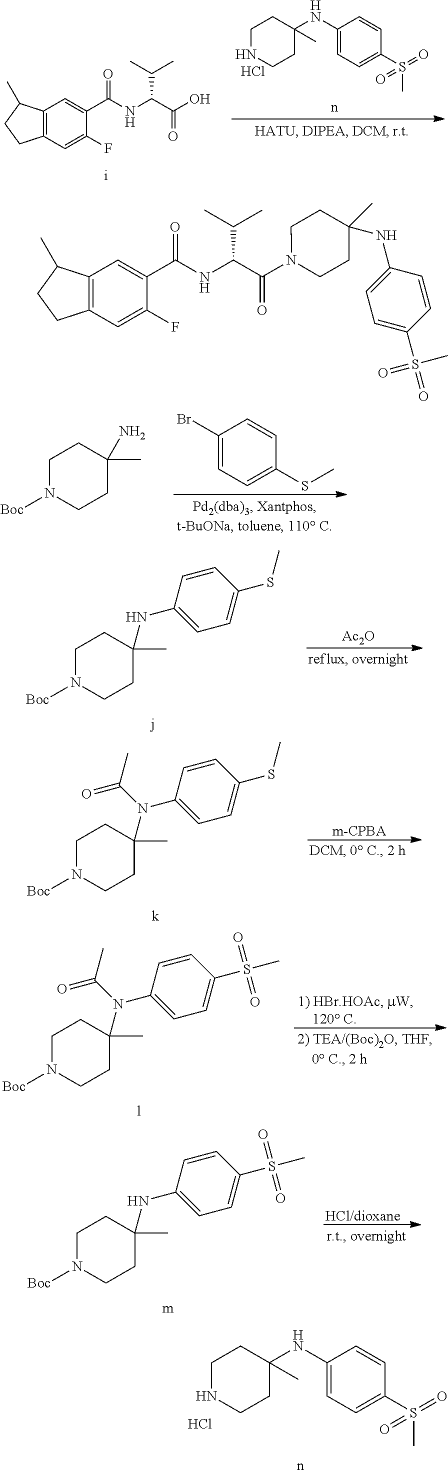

### Ethyl 3-(3-bromo-4-fluorophenyl)but-2-enoate

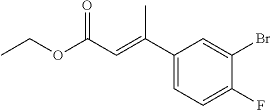 [SMILES: CCOC(=O)/C=C(\C)c1ccc(F)c(Br)c1]

To a solution of 1-(3-bromo-4-fluorophenyl)ethanone (2.17 g, 10 mmol) in toluene (20 mL), was added sodium hydride (575 mg, 15 mmol) at 0° C. and the suspension was stirred at for 0° C. for 2 hours. To the resulting suspension was added dropwise ethyl-2-(diethoxyphosphoryl)acetate (2.69 g, 12 mmol) at 0° C. and the mixture was stirred overnight at 60° C. before the reaction was quenched with ice-water (15 mL). The mixture was extracted with ethyl acetate (3×50 mL). The combined organic layers were dried over anhydrous sodium sulfate and concentrated under reduced pressure. The residue was purified by silica gel column chromatography eluting with ethyl acetate:petroleum ether=1:20 to afford ethyl-3-(3-bromo-4-fluorophenyl)but-2-enoate (1.3 g, 45%) as a yellow oil as a mixture of cis and trans isomers.

LCMS (ESI): m/z=287.1 [M+H]⁺.

¹H-NMR (300 MHz, CDCl₃): δ=1.12-1.16 (t, 3H), 1.30-1.35 (t, 3H), 2.14 (s, 3H), 2.54 (s, 3H), 4.00-4.07 (q, 2H), 4.19-4.26 (q, 2H), 5.92 (s, 1H), 6.08 (s, 1H), 7.09-7.15 (m, 3H), 7.37-7.41 (m, 2H), 7.65-7.68 (m, 1H).

### Ethyl-3-(3-bromo-4-fluorophenyl)butanoate

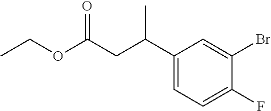 [SMILES: CCOC(=O)CC(C)c1ccc(F)c(Br)c1]

To a solution of ethyl-3-(3-bromo-4-fluorophenyl)but-2-enoate (1.4 g, 4.878 mmol) in methanol (100 mL) was added rhodium(II) acetate (10 mg, cat.). The suspension was stirred at 40° C. for 4 hours under a hydrogen atmosphere (0.4 MPa) before the solid was removed by a filtration. The filtrate was concentrated under reduced pressure and purified by silica gel column chromatography eluting with ethyl acetate:petroleum ether=1:10 to afford ethyl-3-(3-bromo-4-fluorophenyl)butanoate (1.4 g, 99%) as a yellow oil.

LCMS (ESI): m/z=288.2 [M+H]⁺.

¹H-NMR (300 MHz, CD₃OD): δ=1.12-1.17 (t, 3H), 1.26-1.29 (d, 3H, J=9 Hz), 2.57-2.60 (m, 2H), 3.19-3.29 (m, 1H), 4.00-4.07 (m, 2H), 7.08-7.14 (m, 1H), 7.21-7.26 (m, 1H), 7.47-7.50 (m, 1H).

### 3-(3-Bromo-4-fluorophenyl)butanoic acid

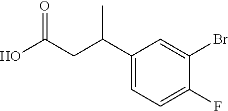

To a solution of ethyl-3-(3-bromo-4-fluorophenyl)butanoate (1.2 g, 4.15 mmol) in tetrahydrofuran (2 mL) was added aqueous lithium hydroxide (10 mL, 2M, 20 mmol). The solution was stirred overnight. The organic solvent was removed under reduced pressure and the pH of the remaining aqueous layer was adjusted to pH 3 with a 10% aqueous hydrochloric acid solution. The resulting suspension was extracted with ethyl acetate (3×50 mL). The combined organic layers were dried over anhydrous sodium sulfate and concentrated under reduced pressure to afford 3-(3-bromo-4-fluorophenyl)butanoic acid (1.05 g, 97%) as a yellow solid.

LCMS (ESI): m/z=261.0 [M+H]⁺.

¹H-NMR (300 MHz, CDCl₃): δ=1.27-1.30 (d, 3H, J=9 Hz), 2.54-2.57 (m, 2H), 3.24-3.27 (m, 1H), 7.01-7.12 (m, 2H), 7.38-7.41 (m, 1H).

### 5-Bromo-6-fluoro-3-methyl-2,3-dihydro-1H-inden-1-one

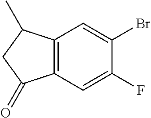

A mixture of 3-(3-bromo-4-fluorophenyl)butanoic acid (1.2 g, 4.6 mmol) and polyphosphoric acid (10 g) was stirred at 110° C. for 2 hours. The reaction was quenched by pouring into ice-water (10 mL). The resulting suspension was extracted with dichloromethane (3×30 mL). The combined organic layers were dried over anhydrous sodium sulfate and concentrated under reduced pressure. The residue was purified by silica gel column chromatography eluting with ethyl acetate:petroleum ether=1:10 to afford 5-bromo-6-fluoro-3-methyl-2,3-dihydro-1H-inden-1-one (530 mg, 47%) as a white solid.

LCMS (ESI): m/z=243.1, 245.1 [M+H]⁺.

¹H-NMR (300 MHz, CDCl₃): δ=1.41-1.43 (d, 3H, J=6 Hz), 2.30-2.37 (m, 1H), 2.95-3.04 (m, 1H), 3.42-3.47 (m, 1H), 7.28-7.43 (m, 1H), 7.42-7.58 (m, 1H).

### Methyl-6-fluoro-3-methyl-1-oxo-2,3-dihydro-1H-indene-5-carboxylate

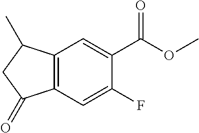 [SMILES: COC(=O)c1cc2c(cc1F)C(=O)CC2C]

To a solution of 5-bromo-6-fluoro-3-methyl-2,3-dihydro-1H-inden-1-one (610 mg, 2.51 mmol) in methanol (20 mL) was added sequentially anhydrous sodium acetate (823 mg, 10 mmol) and [1,1′-bis(diphenylphosphino)ferrocene]palladium(II) chloride (7 mg, 0.01 mmol), the mixture was stirred overnight at 80° C. under a carbon monoxide atmosphere (0.4 MPa). The resulting mixture was concentrated under reduced pressure. The residue was purified by silica gel column chromatography eluting with ethyl acetate:petroleum ether=1:3 to afford methyl-6-fluoro-3-methyl-1-oxo-2,3-dihydro-1H-indene-5-carboxylate (510 mg, 91%) as a light-red solid.

LCMS (ESI): m/z=222.2 [M+H]⁺.

¹H-NMR (300 MHz, CDCl₃): δ=1.41-1.44 (d, 3H, J=6 Hz), 2.31-2.39 (m, 1H), 2.97-3.06 (m, 1H), 3.36-3.51 (m, 1H), 3.97 (s, 3H), 7.40-7.43 (d, 2H, J=9 Hz), 8.02-8.05 (d, 2H, J=9 Hz).

### Methyl-6-fluoro-3-methyl-2,3-dihydro-1H-indene-5-carboxylate

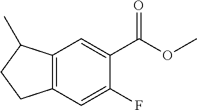

To a solution of methyl-6-fluoro-3-methyl-1-oxo-2,3-dihydro-1H-indene-5-carboxylate (510 mg, 2.29 mmol) in ethanol (20 mL) was added concentrated hydrochloric acid (2 mL) and 5% palladium on carbon (100 mg). The mixture was stirred under hydrogen (0.4 MPa) overnight. The solid was removed by a filtration and the filtrate was concentrated under reduced pressure. The residue was purified by silica gel column chromatography eluting with ethyl acetate:petroleum ether=1:10 to afford methyl 6-fluoro-3-methyl-2,3-dihydro-1H-indene-5-carboxylate (380 mg, 79%) as a colorless oil.

LCMS (ESI): m/z=209.1 [M+H]⁺.

¹H-NMR (300 MHz, CDCl₃): δ=1.25-1.27 (d, 3H, J=6 Hz), 1.60-1.71 (m, 2H), 2.32-2.37 (m, 1H), 2.83-2.88 (m, 2H), 3.14-3.16 (m, 1H), 3.91 (s, 3H), 6.92-6.96 (d, 1H, J=8 Hz), 7.65-7.69 (d, 1H, J=8 Hz).

### 6-Fluoro-3-methyl-2,3-dihydro-1H-indene-5-carboxylic acid

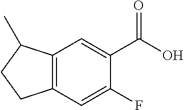

To a solution of methyl-6-fluoro-3-methyl-2,3-dihydro-1H-indene-5-carboxylate (380 mg, 1.83 mmol) in tetrahydrofuran (5 mL) was added aqueous lithium hydroxide (100 mg in 10 mL water, 4 mmol). The mixture was stirred overnight before the pH of the mixture was adjusted to pH 1 with 1.0 M aqueous hydrochloric acid solution. The resulting suspension was extracted with ethyl acetate (3×30 mL). The combine organic layers were dried over anhydrous sodium sulfate and concentrated under reduced pressure to afford 6-fluoro-3-methyl-2,3-dihydro-1H-indene-5-carboxylic acid (310 mg, crude) as a white solid, which was used directly without further purification.

LCMS (ESI): m/z=195.1 [M+H]⁺.

### (2R)-Methyl-2-(6-fluoro-3-methyl-2,3-dihydro-1H-indene-5-carboxamido)-3-methylbutanoate

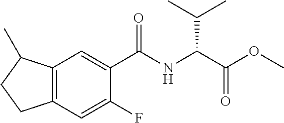 [SMILES: COC(=O)[C@H](NC(=O)c1cc2c(cc1F)CCC2C)C(C)C]

To a suspension of 6-fluoro-3-methyl-2,3-dihydro-1H-indene-5-carboxylic acid (100 mg, 0.515 mmol) in dichloromethane (10 mL) was added sequentially (R)-methyl 2-amino-3-methylbutanoate hydrochloride (68 mg, 0.41 mmol), 1-[bis(dimethylamino)methylene]-1H-1,2,3-triazolo[4,5-b]pyridinium-3-oxide hexafluorophosphate (HATU) ((390 mg, 1.03 mmol) and N,N-diisopropylethylamine (132 mg, 1.03 mmol). The resulting mixture was stirred for 2 hours at room temperature before the reaction was quenched with ice-water (10 mL). The mixture was extracted with dichloromethane (3×10 mL). The combined organic layers were dried over anhydrous sodium sulfate and concentrated under reduced pressure. The residue was purified by prep-TLC (dichloromethane:methanol=15:1) to afford (2R)-methyl-2-(6-fluoro-3-methyl-2,3-dihydro-1H-indene-5-carboxamido)-3-methylbutanoate (150 mg, 94%) as a colorless oil.

LCMS (ESI): m/z=308.2 [M+H]⁺.

¹H-NMR (300 MHz, CDCl₃): δ=0.90-0.95 (m, 6H), 1.18-1.21 (d, 3H, J=6 Hz), 1.51-1.58 (m, 1H), 2.20-2.39 (m, 2H), 2.74-3.07 (m, 3H), 3.68 (s, 3H), 4.68-4.72 (m, 1H), 6.83-6.85 (d, 1H, J=6 Hz), 7.08-7.18 (br, 1H), 7.74-7.76 (d, 1H, J=6 Hz).

### (2R)-2-(6-Fluoro-3-methyl-2,3-dihydro-1H-indene-5-carboxamido)-3-methyl butanoic acid

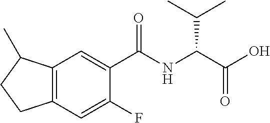 [SMILES: CC1CCc2cc(F)c(C(=O)N[C@@H](C(=O)O)C(C)C)cc21]

To a solution of (2R)-methyl-2-(6-fluoro-3-methyl-2,3-dihydro-1H-indene-5-carboxamido)-3-methylbutanoate (150 mg, 0.488 mmol) in tetrahydrofuran (5 mL) was added aqueous lithium hydroxide (100 mg in 10 mL water, 4 mmol). The mixture was stirred overnight before the pH of the mixture was adjusted pH to 1 with 1.0 M aqueous hydrochloric acid solution. The resulting suspension was extracted with ethyl acetate (3×30 mL). The combined organic layers were dried over anhydrous sodium sulfate and concentrated under reduced pressure to afford (2R)-2-(6-fluoro-3-methyl-2,3-dihydro-1H-indene-5-carboxamido)-3-methylbutanoic acid (80 mg, crude) as a yellow solid, which was used directly without further purification.

LCMS (ESI): m/z=294.3 [M+H]⁺.

### tert-Butyl-4-methyl-4-(4-(methylthio)phenylamino)piperidine-1-carboxylate

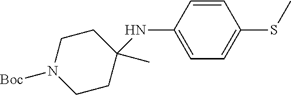 [SMILES: CSc1ccc(NC2(C)CCN(C(=O)OC(C)(C)C)CC2)cc1]

To a solution of tert-butyl 4-amino-4-methylpiperidine-1-carboxylate (22 mg, 0.10 mmol) in toluene (5 mL) was added sequentially (4-bromophenyl)(methyl)sulfane (22 mg, 0.11 mmol), 4,5-bis(diphenylphosphino)-9,9-dimenthxanthene (Xantphos) (5 mg, 0.008 mmol), tris(dibenzylideneacetone)dipalladium(0) (5 mg, 0.005 mmol) and sodium tert-butoxide (20 mg, 0.2 mmol) under argon an atmosphere. The resulting mixture was stirred for 15 minutes before was refluxed at 110° C. overnight. The resulting mixture was concentrated under reduced pressure. The residue was purified by silica gel column chromatography eluting with ethyl acetate:petroleum ether=3:1 to afford tert-butyl-4-methyl-4-(4-(methylthio)phenylamino)piperidine-1-carboxylate (15 mg, 48%) as a yellow oil.

LCMS (ESI): m/z=337.2 [M+H]⁺.

### tert-Butyl-4-methyl-4-(N-(4-(methylthio)phenyl)acetamido)piperidine-1-carboxylate

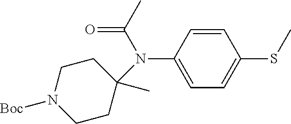 [SMILES: CSc1ccc(N(C(C)=O)C2(C)CCN(C(=O)OC(C)(C)C)CC2)cc1]

A solution of tert-butyl-4-methyl-4-(4-(methylthio)phenylamino)piperidine-1-carboxylate (15 mg, 0.04 mmol) in acetic anhydride (5 mL) was refluxed overnight. The resulting mixture was concentrated under reduced pressure. The residue was purified by silica gel column chromatography eluting with ethyl acetate:petroleum ether=3:1 to afford tert-butyl-4-methyl-4-(N-(4-(methylthio)phenyl)acetamido)piperidine-1-carboxylate (15 mg, 89%) as a yellow oil.

LCMS (ESI): m/z=379.2 [M+H]⁺.

¹H-NMR (300 MHz, CDCl₃): δ=1.42 (s, 9H), 1.57-1.64 (m, 2H), 1.69 (s, 3H), 1.91-1.95 (m, 2H), 2.50 (s, 3H), 2.83-2.96 (m, 2H), 3.72-3.85 (m, 2H), 7.00-7.03 (d, 2H, J=9 Hz), 7.22-7.25 (d, 2H, J=9 Hz).

### tert-Butyl-4-methyl-4-(N-(4-(methylsulfonyl)phenyl)acetamido)piperidine-1-carboxylate

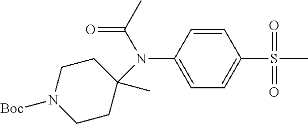 [SMILES: CC(=O)N(c1ccc(S(C)(=O)=O)cc1)C1(C)CCN(C(=O)OC(C)(C)C)CC1]

To a solution of tert-butyl-4-methyl-4-(N-(4-(methylthio)phenyl)acetamido)piperidine-1-carboxylate (22 mg, 0.10 mmol) in dichloromethane (5 mL) was added meta-chloroperoxybenzoic acid (20 mg, 0.2 mmol) at 0° C. After stirring at 0° C. for 1 hour, to the resulting mixture was added dropwise sodium hydroxide (5 mL, 2 M). The resulting mixture was extracted with dichloromethane (3×10 mL). The combined organic layers were concentrated under reduced pressure. The residue was purified by silica gel column chromatography eluting with dichloromethane: methanol=50:1 to afford tert-butyl-4-methyl-4-(N-(4-(methylsu lfonyl)phenyl)acetamido)piperidine-1-carboxylate (15 mg, 48%) as a yellow oil.

LCMS (ESI): m/z=411.2 [M+H]⁺.

¹H-NMR (300 MHz, CDCl₃): δ=1.41 (s, 9H), 1.54-1.57 (m, 2H), 1.61 (s, 3H), 1.65 (s, 3H), 1.91-1.95 (m, 2H), 2.83-2.92 (m, 2H), 3.11 (s, 3H), 3.79-3.84 (m, 2H), 7.32-7.35 (d, 2H, J=9 Hz), 7.96-7.99 (d, 2H, J=9 Hz).

### tert-Butyl-4-methyl-4-(4-(methylsulfonyl)phenylamino)piperidine-1-carboxylate

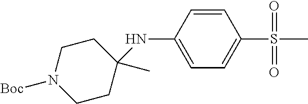 [SMILES: CC1(Nc2ccc(S(C)(=O)=O)cc2)CCN(C(=O)OC(C)(C)C)CC1]

A solution of tert-butyl-4-methyl-4-(N-(4-(methylsulfonyl)phenyl)acetamido)piperidine-1-carboxylate (550 mg, 1.34 mmol) in a 33% solution of hydrogen bromide in acetic acid (5 mL) was heated to 120° C. in a microwave reactor for 20 minutes. The solvents were removed under reduced pressure. The residue was dissolved into tetrahydrofuran (20 mL). To the resulting mixture was added sequentially triethylamine (505 mg, 5 mmol), di-tert-butyl dicarbonate (327 mg, 1.5 mmol) at 000° C. The mixture was stirred overnight before was concentrated under reduced pressure. The residue was purified by silica gel column chromatography eluting with dichloromethane: methanol=50:1 to afford tert-butyl-4-methyl-4-(4-(methylsulfonyl)phenylamino)piperidine-1-carboxylate (220 mg, 45%) as a yellow solid.

LCMS (ESI): m/z=369.2 [M+H]⁺.

¹H-NMR (300 MHz, CDCl₃): δ=1.44 (s, 3H), 1.45 (s, 9H), 1.61-1.72 (m, 2H), 1.88-1.98 (m, 2H), 3.00 (s, 3H), 3.19-3.35 (m, 2H), 3.52-3.77 (m, 2H), 6.71-6.79 (d, 2H, J=9 Hz), 7.64-7.67 (d, 2H, J=9 Hz).

### 4-Methyl-N-(4-(methylsulfonyl)phenyl)piperidin-4-amine hydrochloride

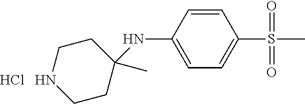 [SMILES: Cl ; CC1(Nc2ccc(S(C)(=O)=O)cc2)CCNCC1]

A solution of tert-butyl-4-methyl-4-(4-(methylsulfonyl)phenylamino)piperidine-1-carboxylate (100 mg, 0.27 mmol) in a 6.0 M solution of hydrochloric acid in dioxane (10 mL) was stirred overnight. The solvent was removed under reduced pressure to afford 4-methyl-N-(4-(methylsulfonyl)phenyl)piperidin-4-amine hydrochloride (70 mg, crude), which was used directly without further purification.

LCMS (ESI): m/z=269.2 [M+H]⁺.

### Example 25

### 6-Fluoro-3-methyl-N—((R)-3-methyl-1-(4-methyl-4-(4-(methylsulfonyl) phenylamino)piperidin-1-yl)-1-oxobutan-2-yl)-2,3-dihydro-1H-indene-5-carboxamide

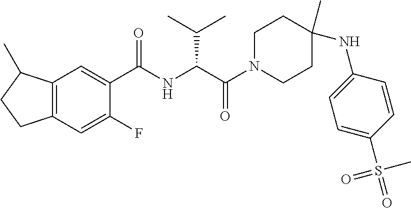 [SMILES: CC1CCc2cc(F)c(C(=O)N[C@@H](C(=O)N3CCC(C)(Nc4ccc(S(C)(=O)=O)cc4)CC3)C(C)C)cc21]

To a suspension of (2R)-2-(6-fluoro-3-methyl-2,3-dihydro-1H-indene-5-carboxamido)-3-methylbutanoic acid (40 mg, 0.132 mmol) in dichloromethane (10 mL) was added sequentially 4-methyl-N-(4-(methylsulfonyl)phenyl)piperidin-4-amine hydro chloride (39 mg, 0.135 mmol), 1-[bis(dimethylamino)methylene]-1H-1,2,3-triazolo[4,5-b]pyridinium-3-oxide hexafluorophosphate (HATU) (99 mg, 0.263 mmol) and N,N-diisopropylethylamine (34 mg, 0.263 mmol). The resulting mixture was stirred for 2 hours at room temperature before the reaction was quenched with ice-water (10 mL). The mixture was extracted with dichloromethane (3×10 mL). The combined organic layers were dried over anhydrous sodium sulfate and concentrated under reduced pressure. The residue was purified by prep-HPLC (gradient: 95% water, 5% acetonitrile, 30-50 min gradient to 40% water, 60% acetonitrile) to afford 6-fluoro-3-methyl-N—((R)-3-methyl-1-(4-methyl-4-(4-(methylsulfonyl)phenylamino)piperidin-1-yl)-1-oxobutan-2-yl)-2,3-dihydro-1H-indene-5-carboxamide (Example 25) as a white solid (20 mg, 28%).

LCMS (ESI): m/z=544.2 [M+H]⁺.

¹H-NMR (400 MHz, CD₃OD): δ=1.00-1.06 (m, 6H), 1.28-1.33 (m, 3H), 1.46-1.5 (d, 3H, J=16 Hz), 1.63-1.70 (m, 3H), 2.14-2.39 (m, 4H), 2.90-2.97 (m, 2H), 3.03-3.05 (d, 3H, J=6 Hz), 3.19-3.21 (m, 2H), 3.40-4.25 (m, 3H), 4.95-5.04 (m, 1H), 6.88-6.93 (m, 2H), 7.05-7.09 (m, 1H), 7.56-7.65 (m, 3H), 8.05-8.16 (m, 1H).

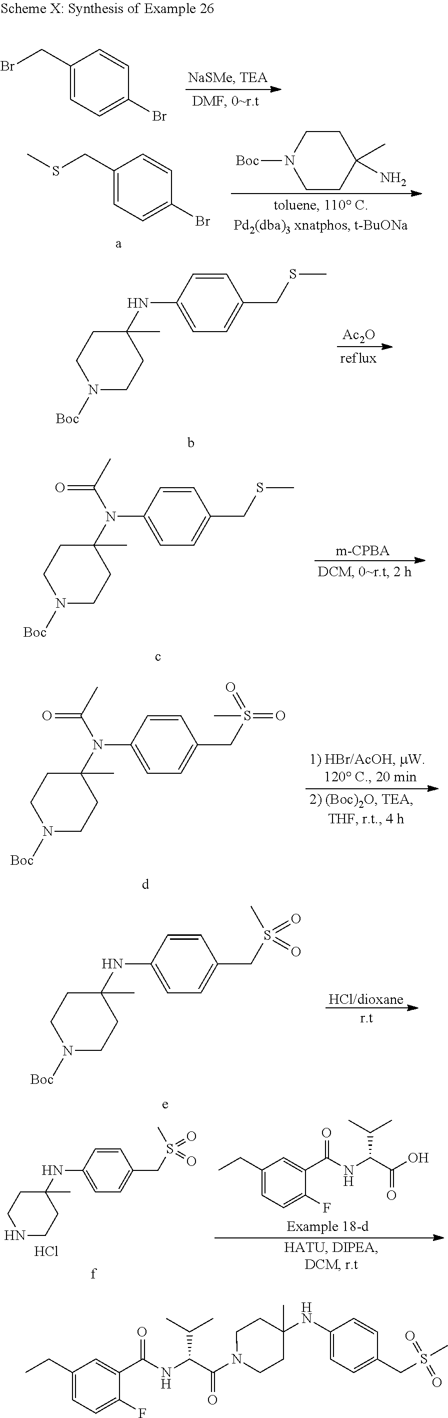

### (4-Bromobenzyl)(methyl)sulfane

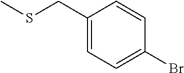

To a solution of 1-bromo-4-(bromomethyl)benzene (1.25 g, 5 mmol) in N,N-dimethylformamide (5 mL) was added triethylamine (1.1 g, 10 mmol) at 0° C. and the reaction was stirred overnight before was quenched with water (20 mL). The resulting mixture was extracted with ethyl acetate (3×20 mL). The combined organic layers were dried over anhydrous sodium sulfate and concentrated under reduced pressure. The residue was purified by silica gel column chromatography eluting with ethyl acetate:petroleum ether=1:20 to afford (4-bromobenzyl)(methyl)sulfane (840 mg, 77%) as a colorless oil.

GCMS (E/): m/z=216, 218 [M].

¹H-NMR (400 MHz, CDCl₃): δ=1.99 (s, 3H), 3.63 (s, 3H), 7.17-7.20 (d, 2H, J=6 Hz), 7.43-7.46 (d, 2H, J=6 Hz).

### tert-Butyl-4-methyl-4-(4-(methylthiomethyl)phenylamino)piperidine-1-carboxylate

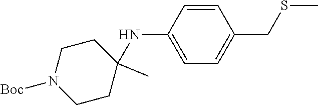 [SMILES: CSCc1ccc(NC2(C)CCN(C(=O)OC(C)(C)C)CC2)cc1]

To a solution of tert-butyl-4-amino-4-methylpiperidine-1-carboxylate (214 mg, 1 mmol) in toluene (30 mL) was added sequentially (4-bromobenzyl)(methyl)sulfane (217 mg, 1 mmol), 4,5-bis(diphenylphosphino)-9,9-dimenthxanthene (Xantphos) (100 mg, 0.19 mmol), tris(dibenzylideneacetone)dipalladium(0) (Pd₂(dba)₃) (50 mg, 0.05 mmol) and sodium tert-butoxide (192 mg, 2 mmol) under an argon atmosphere. The resulting mixture was stirred for 15 minutes before was refluxed at 110° C. overnight. The resulting mixture was concentrated under reduced pressure. The residue was purified by silica gel column chromatography eluting with ethyl acetate:petroleum ether=1:3 to afford tert-butyl-4-methyl-4-(4-(methylthiomethyl)phenylamino)piperidine-1-carboxylate (120 mg, 34%) as a yellow oil.

LCMS (ESI): m/z=351.2 [M+H]⁺.

¹H-NMR (400 MHz, CDCl₃): δ=1.35 (s, 3H), 1.46 (s, 9H), 1.54-1.72 (m, 2H), 1.86-1.95 (m, 2H), 2.00 (s, 3H), 2.25-3.45 (m, 2H), 3.60 (s, 2H), 6.69-6.72 (d, 2H, J=6 Hz), 7.08-7.11 (d, 2H, J=6 Hz).

### tert-Butyl-4-methyl-4-(N-(4-(methylthiomethyl)phenyl)acetamido)piperidine-1-carboxylate

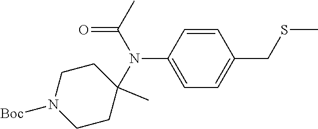 [SMILES: CSCc1ccc(N(C(C)=O)C2(C)CCN(C(=O)OC(C)(C)C)CC2)cc1]

A solution of tert-butyl-4-methyl-4-(4-(methylthiomethyl)phenylamino)piperidine-1-carboxylate (120 mg, 0.34 mmol) in acetic anhydride (10 mL) was heated at reflux overnight. The resulting mixture was concentrated under reduced pressure. The residue was purified by silica gel column chromatography eluting with ethyl acetate:petroleum ether=3:1 to afford tert-butyl-4-methyl-4-(N-(4-(methylthiomethyl)phenyl)acetamido)piperidine-1-carboxylate (70 mg, crude) as a yellow oil, which was used directly without further purification.

LCMS (ESI): m/z=393.2 [M+H]⁺.

### tert-Butyl-4-methyl-4-(N-(4-(methylsulfonylmethyl)phenyl)acetamido)piperidine-1-carboxylate

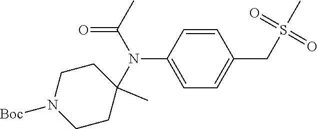 [SMILES: CC(=O)N(c1ccc(CS(C)(=O)=O)cc1)C1(C)CCN(C(=O)OC(C)(C)C)CC1]

To a solution of tert-butyl-4-methyl-4-(N-(4-(methylthiomethyl)phenyl)acetamido)piperidine-1-carboxylate (70 mg, crude) in dichloromethane (10 mL) was added meta-chloroperoxybenzoic acid (70 mg, 0.4 mmol) at 00° C. After stirring at 00° C. for 1 hour, the resulting mixture was added dropwise a 2.0 M aqueous sodium hydroxide solution (5 mL). The resulting mixture was extracted with dichloromethane (3×10 mL). The combined organic layers were concentrated under reduced pressure to afford tert-butyl-4-methyl-4-(N-(4-(methylsulfonylmethyl)phenyl)acetamido)piperidine-1-carboxylate (100 mg, crude) as a yellow oil, which was used directly without further purification.

LCMS (ESI): m/z=425.2 [M+H]⁺.

### tert-Butyl-4-methyl-4-(4-(methylsulfonylmethyl)phenylamino)piperidine-1-carboxylate

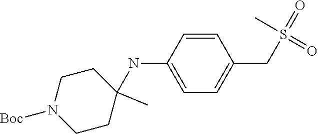 [SMILES: CC1(Nc2ccc(CS(C)(=O)=O)cc2)CCN(C(=O)OC(C)(C)C)CC1]

A solution of tert-butyl-4-methyl-4-(N-(4-(methylsulfonylmethyl)phenyl)acetamido)piperidine-1-carboxylate (100 mg, crude) in a 33% solution of hydrogen bromide in acetic acid (5 mL) was heated to 120° C. with microwave reactor for 20 minutes. The solvents were removed under reduced pressure. The residue was dissolved into tetrahydrofuran (20 mL). To the resulting mixture was added sequentially triethylamine (505 mg, 5 mmol), di-tert-butyl dicarbonate (327 mg, 1.5 mmol) at 000° C. The mixture was stirred overnight before it was concentrated under reduced pressure. The residue was purified by silica gel column chromatography eluting with dichloromethane: methanol=50:1 to afford tert-butyl-4-methyl-4-(4-(methylsulfonylmethyl)phenylamino) piperidine-1-carboxylate (70 mg, crude) as a yellow solid, which was used directly without further purification.

LCMS (ESI): m/z=383.2 [M+H]⁺.

### 4-Methyl-N-(4-(methylsulfonylmethyl)phenyl)piperidin-4-amine hydrochloride

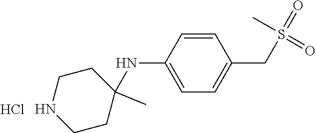 [SMILES: Cl ; CC1(Nc2ccc(CS(C)(=O)=O)cc2)CCNCC1]

A solution of tert-butyl 4-methyl-4-(4-(methylsulfonylmethyl)phenylamino)piperidine-1-carboxylate (100 mg, 0.27 mmol) in a 6.0 M solution of hydrochloric acid in dioxane (10 mL, 6 M) was stirred overnight. The solvent was removed under reduced pressure to afford 4-methyl-N-(4-(methylsulfonylmethyl)phenyl)piperidin-4-amine hydrochloride (48 mg, crude), which was used directly without further purification.

LCMS (ESI): m/z=283.2 [M+H]⁺.

### Example 26

### (R)-5-Ethyl-2-fluoro-N-(3-methyl-1-(4-methyl-4-(4-(methylsulfonylmethyl)phenylamino)piperidin-1-yl)-1-oxobutan-2-yl)benzamide

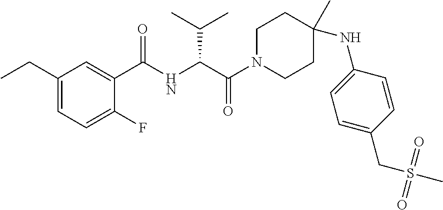 [SMILES: CCc1ccc(F)c(C(=O)N[C@@H](C(=O)N2CCC(C)(Nc3ccc(CS(C)(=O)=O)cc3)CC2)C(C)C)c1]

To a suspension of (R)-2-(5-ethyl-2-fluorobenzamido)-3-methylbutanoic acid (prepared as described in Example 18-d) (34 mg, 0.13 mmol) in dichloromethane (10 mL) was added sequentially 4-methyl-N-(4-(methylsulfonylmethyl)phenyl)piperidin-4-amine hydrochloride salt (48 mg, crude), 1-[bis(dimethylamino)methylene]-1H-1,2,3-triazolo[4,5-b]pyridinium-3-oxide hexafluorophosphate (HATU) (99 mg, 0.263 mmol) and N,N-diisopropylethylamine (34 mg, 0.263 mmol). The resulting mixture was stirred for 2 hours at room temperature before the reaction was quenched with ice-water (10 mL). The mixture was extracted with dichloromethane (3×10 mL). The combined organic layers were dried over anhydrous sodium sulfate and concentrated under reduced pressure. The residue was purified by prep-TLC (dichloromethane:methanol=15:1 to afford (R)-5-ethyl-2-fluoro-N-(3-methyl-1-(4-methyl-4-(4-(methylsulfonylmethyl)phenylamino)piperidin-1-yl)-1-oxobutan-2-yl)benzamide (Example 26) as a white solid (50 mg, 71%).

LCMS (ESI): m/z=532.2 [M+H]⁺.

¹H-NMR (400 MHz, CD₃OD): δ=1.02-1.07 (m, 6H), 1.24-1.29 (m, 3H), 1.48-1.51 (d, 3H, J=14 Hz), 1.79-2.25 (m, 5H), 2.68-2.72 (m, 2H), 2.95-2.96 (m, 2H), 3.04-3.17 (m, 1H), 3.38-3.69 (m, 2H), 4.03-4.46 (m, 2H), 4.48-4.51 (d, 2H, J=12 Hz), 4.92-4.99 (m, 1H), 7.15-7.30 (m, 2H), 7.38-7.43 (m, 2H), 7.54-7.63 (m, 3H).

### Example 27

### Prepared Using a Method Analogous to Example 26

### (R)-2-Fluoro-N-(3-methyl-1-(4-methyl-4-(4-(methylsulfonyl)phenylamino)piperidin-1-yl)-1-oxobutan-2-yl)-5-(trifluoromethyl)benzamide

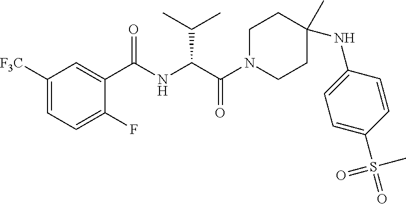 [SMILES: CC(C)[C@@H](NC(=O)c1cc(C(F)(F)F)ccc1F)C(=O)N1CCC(C)(Nc2ccc(S(C)(=O)=O)cc2)CC1]

6.1 mg, yield: 10%, appearance: white solid.

LCMS (ESI): m/z=558.2 [M+H]⁺.

¹H-NMR (400 MHz, CD₃OD): δ=1.01-1.07 (m, 6H), 1.46-1.50 (d, 3H, J=15 Hz), 1.68-1.82 (m, 2H), 2.14-2.26 (m, 3H), 3.04-3.05 (d, 3H, J=4 Hz), 3.15-3.32 (m, 1H), 3.34-4.26 (m, 3H), 4.89-4.99 (m, 1H), 6.90-6.93 (m, 2H), 7.46-7.50 (m, 1H), 7.62-7.65 (m, 2H), 7.89-8.06 (m, 2H), 8.49-8.60 (m, 1H).

### Example 28

### Prepared Using a Method Analogous to Example 26

### (R)-5-(Difluoromethoxy)-2-fluoro-N-(3-methyl-1-(4-methyl-4-(4-(methyl sulfonyl)phenylamino)piperidin-1-yl)-1-oxobutan-2-yl)benzamide

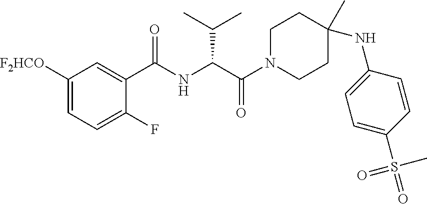 [SMILES: CC(C)[C@@H](NC(=O)c1cc(OC(F)F)ccc1F)C(=O)N1CCC(C)(Nc2ccc(S(C)(=O)=O)cc2)CC1]

18.8 mg, yield: 44%, appearance: white solid.

LCMS (ESI): m/z=556.1 [M+H]⁺.

¹H-NMR (400 MHz, CD₃OD): δ=1.00-1.07 (m, 6H), 1.46-1.50 (d, 3H, J=16 Hz), 1.1.64-1.82 (m, 2H), 2.14-2.18 (m, 3H), 3.04-3.05 (d, 3H, J=3 Hz), 3.17-3.28 (m, 1H), 3.39-4.23 (m, 4H), 4.94-4.98 (m, 1H), 6.66-7.05 (m, 3H), 7.27-7.36 (m, 2H), 7.52-7.54 (m, 1H), 7.62-7.66 (m, 2H).

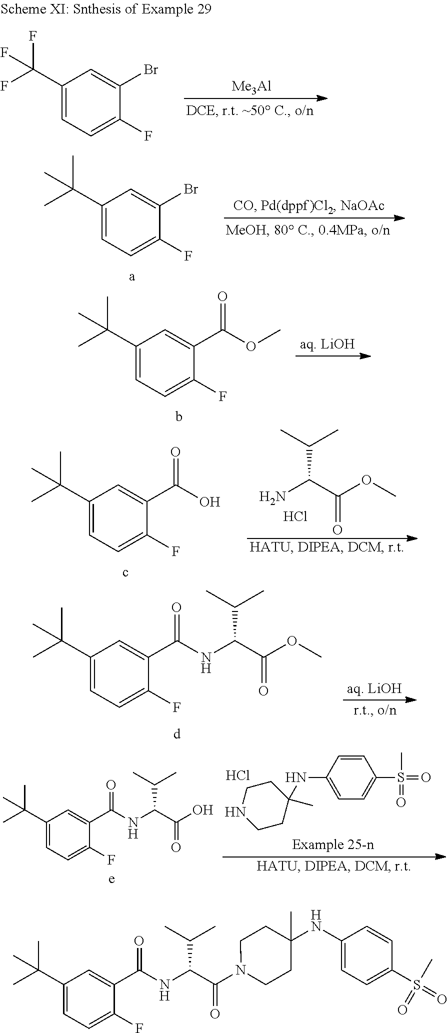

### 2-Bromo-4-tert-butyl-1-fluorobenzene

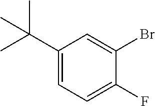

To a solution of 2-bromo-1-fluoro-4-(trifluoromethyl)benzene (1.5 g, 6.17 mmol) in 1,2-dichloroethane (10 mL) was added dropwise a 1.0 M solution of trimethylaluminum in hexane (30 mL, 30 mmol). The mixture was stirred for 1 hour before it was heated to 50° C. overnight. The resulting mixture was quenched with water (3 mL) and concentrated under reduced pressure, The residue was purified by silica gel column chromatography eluting with dichloromethane: methanol=200:1 to afford 2-bromo-4-tert-butyl-1-fluorobenzene (300 mg, crude) as a colorless oil, which was used directly without further purification.

GCMS (ESI): m/z=230 [M]⁺.

### Methyl-5-tert-butyl-2-fluorobenzoate

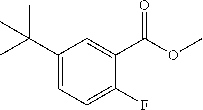

To a solution of 2-bromo-4-tert-butyl-1-fluorobenzene (180 mg, 0.78 mmol) in methanol (20 mL) was added sequentially anhydrous sodium acetate (164 mg, 2 mmol) and [1,1′-bis(diphenylphosphino)ferrocene]palladium(II) chloride (7 mg, 0.01 mmol), the mixture was stirred overnight at 80° C. under a carbon monoxide atmosphere (0.4 MPa). The resulting mixture was concentrated under reduced pressure. The residue was purified by silica gel column chromatography eluting with ethyl acetate:petroleum ether=1:3 to afford methyl-5-tert-butyl-2-fluorobenzoate (110 mg, 67%) as a colorless oil.

LCMS (ESI): m/z=211.2 [M+H]⁺.

### 5-tert-Butyl-2-fluorobenzoic acid

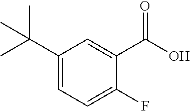

To a solution of methyl-5-tert-butyl-2-fluorobenzoate (110 mg, 0.523 mmol) in tetrahydrofuran (5 mL) was added aqueous lithium hydroxide (100 mg in 10 mL water, 4 mmol). The mixture was stirred overnight before the pH of the mixture was adjusted to pH 1 with 1.0 M aqueous hydrochloric acid solution. The resulting suspension was extracted with ethyl acetate (3×30 mL). The combine organic layers were dried over anhydrous sodium sulfate and concentrated under reduced pressure to afford 5-tert-butyl-2-fluorobenzoic acid (80 mg, 78%) as a white solid.

LCMS (ESI): m/z=197.1 [M+H]⁺.

### (R)-Methyl-2-(5-tert-butyl-2-fluorobenzamido)-3-methylbutanoate

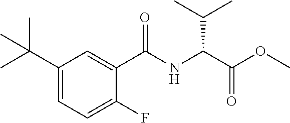 [SMILES: COC(=O)[C@H](NC(=O)c1cc(C(C)(C)C)ccc1F)C(C)C]

To a suspension of 5-tert-butyl-2-fluorobenzoic acid (80 mg, 0.4 mmol) in dichloromethane (10 mL) was added sequentially (R)-methyl-2-amino-3-methylbutanoate hydrochloride (68 mg, 0.40 mmol), 1-[bis(dimethylamino)methylene]-1H-1,2,3-triazolo[4,5-b]pyridinium-3-oxide hexafluorophosphate (HATU) (390 mg, 1.03 mmol) and N,N-diisopropylethylamine (132 mg, 1.03 mmol). The resulting mixture was stirred for 2 hours before the reaction was quenched with ice-water (10 mL). The mixture was extracted with dichloromethane (3×10 mL). The combined organic layers were dried over anhydrous sodium sulfate and concentrated under reduced pressure. The residue was purified by silica gel column chromatography eluting with ethyl acetate:petroleum ether=1:3 to afford (R)-methyl-2-(5-tert-butyl-2-fluorobenzamido)-3-methylbutanoate (70 mg, 55%) as a white solid.

LCMS (ESI): m/z=310.2 [M+H]⁺.

### (R)-2-(5-tert-Butyl-2-fluorobenzamido)-3-methylbutanoic acid

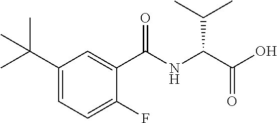 [SMILES: CC(C)[C@@H](NC(=O)c1cc(C(C)(C)C)ccc1F)C(=O)O]

To a solution of (R)-methyl-2-(5-tert-butyl-2-fluorobenzamido)-3-methylbutanoate (70 mg, 0.22 mmol) in tetrahydrofuran (5 mL) was added aqueous lithium hydroxide (100 mg in 10 mL water, 4 mmol). The mixture was stirred overnight before the pH of the mixture was adjusted to pH 1 with 1.0 M aqueous hydrochloric acid solution. The resulting suspension was extracted with ethyl acetate (3×30 mL). The combine organic layers were dried over anhydrous sodium sulfate and concentrated under reduced pressure to afford (R)-2-(5-tert-butyl-2-fluorobenzamido)-3-methylbutanoic acid (50 mg, 75%) as a white solid.

LCMS (ESI): m/z=296.2 [M+H]⁺.

¹H-NMR (400 MHz, CD₃OD): δ=1.02-1.06 (t, 6H), 1.34 (s, 9H), 2.26-2.33 (m, 1H), 4.55-4.57 (m, 1H), 7.11-7.17 (m, 1H), 7.56-7.62 (m, 1H), 7.79-7.81 (m, 1H).

### Example 29

### (R)-5-tert-Butyl-2-fluoro-N-(3-methyl-1-(4-methyl-4-(4-(methylsulfonyl) phenylamino)piperidin-1-yl)-1-oxobutan-2-yl)benzamide

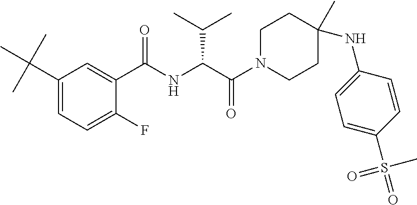 [SMILES: CC(C)[C@@H](NC(=O)c1cc(C(C)(C)C)ccc1F)C(=O)N1CCC(C)(Nc2ccc(S(C)(=O)=O)cc2)CC1]

To a suspension of (R)-2-(5-tert-butyl-2-fluorobenzamido)-3-methylbutanoic acid (50 mg, 0.17 mmol) in dichloromethane (10 mL) was added sequentially 4-methyl-N-(4-(methylsulfonyl)phenyl)piperidin-4-amine hydrochloride (prepared as described in Example 25-n) (48 mg, crude), 1-[bis(dimethylamino)methylene]-1H-1,2,3-triazolo[4,5-b]pyridinium-3-oxide hexafluorophosphate (HATU) (99 mg, 0.263 mmol) and N,N-diisopropylethylamine (34 mg, 0.263 mmol). The resulting mixture was stirred for 2 hours at room temperature before the reaction was quenched with ice-water (10 mL). The mixture was extracted with dichloromethane (3×10 mL). The combined organic layers were dried over anhydrous sodium sulfate and concentrated under reduced pressure. The residue was purified by prep-TLC (dichloromethane:methanol=15:1 to afford (R)-5-tert-butyl-2-fluoro-N-(3-methyl-1-(4-methyl-4-(4-(methylsulfonyl)phenylamino)piperidin-1-yl)-1-oxobutan-2-yl)benzamide (Example 29) as a white solid (20 mg, 22%).

LCMS (ESI): m/z=546.2 [M+H]⁺.

¹H-NMR (400 MHz, CD₃OD): δ=1.01-1.07 (m, 6H), 1.31-1.37 (m, 9H), 1.46-1.51 (d, 3H, J=17 Hz), 1.60-1.90 (m, 2H), 2.08-2.33 (m, 3H), 3.04-3.05 (m, 3H), 3.22-3.33 (m, 1H), 3.40-4.26 (m, 3H), 4.98-4.99 (m, 1H), 6.90-6.93 (m, 2H), 7.16-7.20 (m, 1H), 7.62-7.66 (m, 3H), 7.81-7.84 (m, 1H).

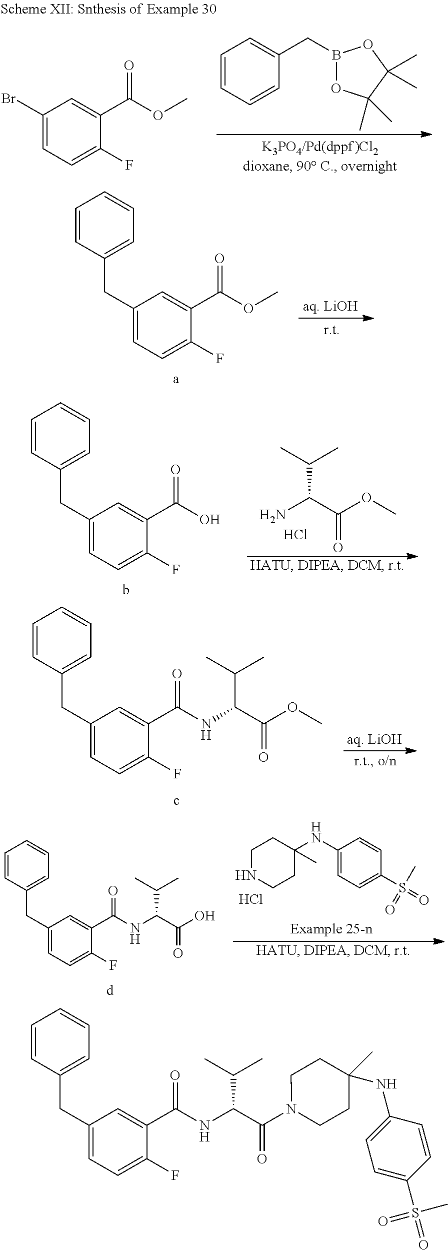

### Methyl-5-benzyl-2-fluorobenzoate

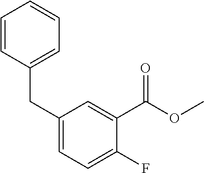 [SMILES: COC(=O)c1cc(Cc2ccccc2)ccc1F]

To a solution of methyl-5-bromo-2-fluorobenzoate (350 mg, 1.5 mmol) in dioxane (20 mL), was added sequentially 2-benzyl-4,4,5,5-tetramethyl-1,3,2-dioxaborolane (327 mg, 1.5 mmol), anhydrous potassium phosphate (955 mg, 4.5 mmol) and [1,1′-bis(diphenylphosphino)ferrocene]palladium(II) chloride (7 mg, 0.01 mmol) under an argon atmosphere. The mixture was stirred at 90° C. overnight before dioxane was concentrated under reduced pressure. The residue was purified by silica gel column chromatography eluting with ethyl acetate:petroleum ether=1:10 to afford methyl-5-benzyl-2-fluorobenzoate (350 mg, crude) as a colorless oil, which was used directly without further purification.

LCMS (ESI): m/z=245.1 [M+H]⁺.

### 5-Benzyl-2-fluorobenzoic acid

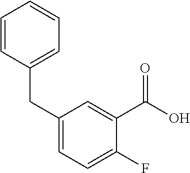 [SMILES: O=C(O)c1cc(Cc2ccccc2)ccc1F]

To a solution of methyl 5-benzyl-2-fluorobenzoate (350 mg, crude) in tetrahydrofuran (1 mL) was added lithium hydroxide (100 mg in 10 mL water, 4 mmol). The mixture was stirred overnight. The organic solvent was removed under reduced pressure and the pH of the remaining aqueous layer was adjusted to pH 1 with 1.0 M aqueous hydrochloric acid solution. The suspension was extracted with ethyl acetate (3×30 mL). The combined organic layers were concentrated under reduced pressure to afford 5-benzyl-2-fluorobenzoic acid (400 mg, crude) as a white solid, which was used directly without further purification.

LCMS (ESI): m/z=231.1 [M+H]⁺.

### (R)-Methyl 2-(5-benzyl-2-fluorobenzamido)-3-methylbutanoate

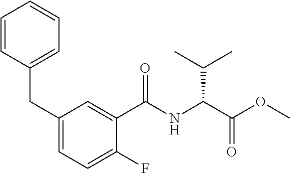 [SMILES: COC(=O)[C@H](NC(=O)c1cc(Cc2ccccc2)ccc1F)C(C)C]

To a suspension of 5-benzyl-2-fluorobenzoic acid (400 mg, crude) in dichloromethane (10 mL) was added sequentially (R)-methyl 2-amino-3-methylbutanoate hydrochloride (306 mg, 1.74 mmol), 1-[bis(dimethylamino)methylene]-1H-1,2,3-triazolo[4,5-b]pyridinium-3-oxide hexafluorophosphate (HATU) (1320 mg, 3.48 mmol) and N,N-diisopropylethylamine (500 mg, 3.48 mmol). The resulting mixture was stirred for 2 hours before the reaction was quenched with ice-water (10 mL). The mixture was extracted with dichloromethane (3×10 mL). The combined organic layers were dried over anhydrous sodium sulfate and concentrated under reduced pressure. The residue was purified by silica gel column chromatography eluting with ethyl acetate:petroleum ether=1:3 to afford (R)-methyl-2-(5-benzyl-2-fluorobenzamido)-3-methylbutanoate (350 mg, crude) as a white solid, which was used directly without further purification.

LCMS (ESI): m/z=344.2 [M+H]⁺.

### (R)-2-(5-Benzyl-2-fluorobenzamido)-3-methylbutanoic acid

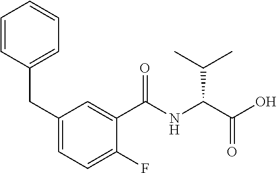 [SMILES: CC(C)[C@@H](NC(=O)c1cc(Cc2ccccc2)ccc1F)C(=O)O]

To a solution of (R)-methyl-2-(5-benzyl-2-fluorobenzamido)-3-methylbutanoate (350 mg, crude) in tetrahydrofuran (5 mL) was added lithium hydroxide (100 mg in 10 mL water, 4 mmol). The mixture was stirred overnight. The organic solvent was removed under reduced pressure and the pH of the remaining aqueous layer was adjusted to pH 1 with 1.0 M aqueous hydrochloric acid solution. The suspension was extracted with ethyl acetate (3×30 mL). The combined organic layers were concentrated under reduced pressure to afford (R)-2-(5-benzyl-2-fluorobenzamido)-3-methylbutanoic acid (170 mg, 51% over two steps) as a white solid.

LCMS (ESI): m/z=330.1 [M+H]⁺.

¹H-NMR (400 MHz, CD₃OD): δ=1.04-1.08 (m, 6H), 2.23-2.45 (m, 1H), 3.99 (s, 2H), 4.74-4.86 (m, 1H), 6.97-7.26 (m, 8H).

### Example 30

### (R)-5-Benzyl-2-fluoro-N-(3-methyl-1-(4-methyl-4-(4-(methylsulfonyl)phenyl amino)piperidin-1-yl)-1-oxobutan-2-yl)benzamide

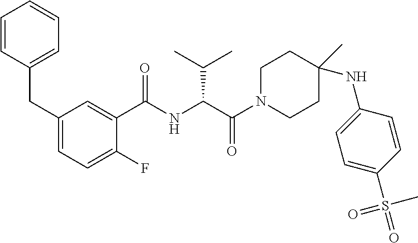 [SMILES: CC(C)[C@@H](NC(=O)c1cc(Cc2ccccc2)ccc1F)C(=O)N1CCC(C)(Nc2ccc(S(C)(=O)=O)cc2)CC1]

To a suspension of (R)-2-(5-benzyl-2-fluorobenzamido)-3-methylbutanoic acid (51 mg, 0.17 mmol) in dichloromethane (10 mL) was added sequentially 4-methyl-N-(4-(methylsulfonyl)phenyl)piperidin-4-amine hydrochloride (prepared as described in Example 25-n) (48 mg, crude), 1-[bis(dimethylamino)methylene]-1H-1,2,3-triazolo[4,5-b]pyridinium-3-oxide hexafluorophosphate (HATU) (99 mg, 0.263 mmol) and N,N-diisopropylethylamine (34 mg, 0.263 mmol). The resulting mixture was stirred for 2 hours at room temperature before the reaction was quenched with ice-water (10 mL). The mixture was extracted with dichloromethane (3×10 mL). The combined organic layers were dried over anhydrous sodium sulfate and concentrated under reduced pressure. The residue was purified by prep-TLC (dichloromethane:methanol=15:1 to afford ((R)-5-benzyl-2-fluoro-N-(3-methyl-1-(4-methyl-4-(4-(methylsulfonyl)phenylamino)piperidin-1-yl)-1-oxobutan-2-yl)benzamide (Example 30) as a white solid (18 mg, 22%).

LCMS (ESI): m/z=580.3 [M+H]⁺.

¹H-NMR (400 MHz, CD₃OD): δ=0.98-1.05 (m, 6H), 1.44-1.49 (d, 3H, J=20 Hz), 1.62-1.81 (m, 2H), 2.12-2.13 (m, 3H), 3.02-3.04 (d, 3H, J=8 Hz), 3.19-3.20 (m, 1H), 3.68-4.20 (m, 5H), 4.89-4.97 (m, 1H), 6.88-6.92 (m, 2H), 7.12-7.40 (m, 7H), 7.57-7.65 (m, 3H).

### Example 31

### Prepared Using a Method Analogous to Example 30

### (R)-2-Fluoro-5-isobutyl-N-(3-methyl-1-(4-methyl-4-(4-(methylsulfonyl)phenylamino)piperidin-1-yl)-1-oxobutan-2-yl)benzamide

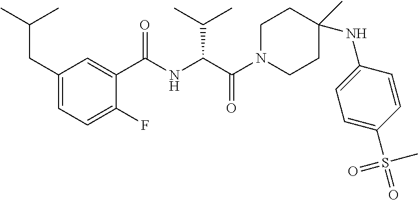 [SMILES: CC(C)Cc1ccc(F)c(C(=O)N[C@@H](C(=O)N2CCC(C)(Nc3ccc(S(C)(=O)=O)cc3)CC2)C(C)C)c1]

25.1 mg, yield: 42%, appearance: white solid.

LCMS (ESI): m/z=546.2 [M+H]⁺.

¹H-NMR (400 MHz, CD₃OD): δ=0.92-0.94 (m, 6H), 1.03-1.08 (m, 6H), 1.45-1.49 (m, 3H), 1.62-1.85 (m, 3H), 2.13-2.28 (m, 3H), 2.50-2.54 (m, 2H), 3.03-3.04 (m, 3H), 3.18-3.57 (m, 2H), 3.69-4.20 (m, 2H), 4.97-5.00 (m, 1H), 6.91 (m, 2H), 7.11-7.18 (m, 1H), 7.32-7.37 (m, 1H), 7.53-7.58 (m, 1H), 7.61-7.65 (m, 2H).

### Example 32

### Prepared Using a Method Analogous to Example 30

### (R)-4-Fluoro-N-(3-methyl-1-(4-methyl-4-(4-(methylsulfonyl)phenylamino)piperidin-1-yl)-1-oxobutan-2-yl)biphenyl-3-carboxamide

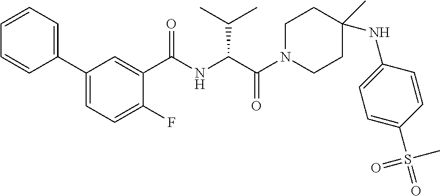 [SMILES: CC(C)[C@@H](NC(=O)c1cc(-c2ccccc2)ccc1F)C(=O)N1CCC(C)(Nc2ccc(S(C)(=O)=O)cc2)CC1]

25.1 mg, yield: 42%, appearance: white solid.

LCMS (ESI): m/z=566.2 [M+H]⁺.

¹H-NMR (400 MHz, CD₃OD): δ=1.02-1.08 (m, 6H), 1.45-1.49 (m, 3H), 1.64-1.87 (m, 2H), 2.12-2.29 (m, 3H), 3.01-3.04 (m, 3H), 3.20-4.19 (m, 4H), 4.99-5.01 (m, 1H), 6.91-6.93 (m, 2H), 7.30-7.49 (m, 4H), 7.60-7.66 (m, 4H), 7.78-7.83 (m, 1H), 7.96-8.01 (m, 1H).

### Example 33

### Prepared Using a Method Analogous to Example 30

### (R)-5-Cyclopropyl-2-fluoro-N-(3-methyl-1-(4-methyl-4-(4-(methylsulfonyl)phenylamino)piperidin-1-yl)-1-oxobutan-2-yl)benzamide

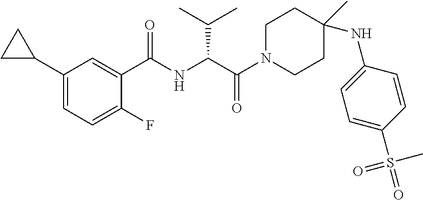 [SMILES: CC(C)[C@@H](NC(=O)c1cc(C2CC2)ccc1F)C(=O)N1CCC(C)(Nc2ccc(S(C)(=O)=O)cc2)CC1]

30 mg, yield: 56%, appearance: white solid.

LCMS (ESI): m/z=530.2 [M+H]⁺.

¹H-NMR (400 MHz, CD₃OD): δ=0.66-0.73 (m, 2H), 0.97-1.07 (m, 6H), 1.38-1.41 (m, 2H), 1.48 (d, J=16.3 Hz, 3H), 1.63-1.85 (m, 2H), 1.94-2.02 (m, 1H), 2.13-2.27 (m, 3H), 3.04 (d, J=4.1 Hz, 3H), 3.18-3.26 (m, 1H), 3.42-3.57 (m, 1H), 3.92-4.03 (m, 1H), 4.25-4.22 (m, 1H), 4.95-5.02 (m, 1H), 6.87-6.93 (m, 2H), 7.07-7.16 (m, 1H), 7.24-7.32 (m, 1H), 7.43-7.49 (m, 1H), 7.61-7.66 (m, 2H).

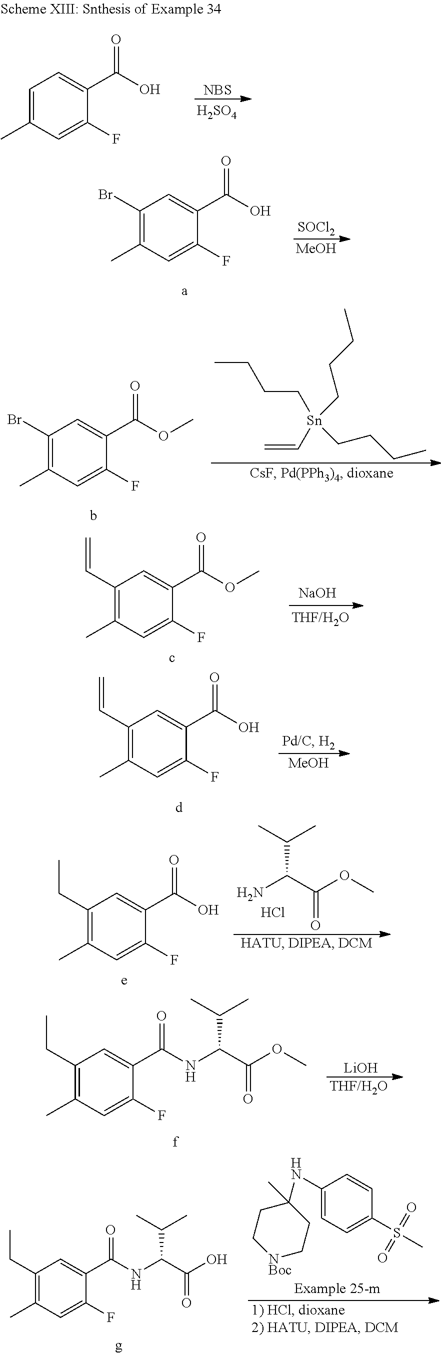

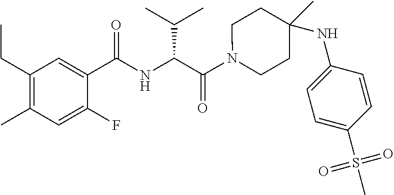 [SMILES: CCc1cc(C(=O)N[C@@H](C(=O)N2CCC(C)(Nc3ccc(S(C)(=O)=O)cc3)CC2)C(C)C)c(F)cc1C]

### 5-Bromo-2-fluoro-4-methylbenzoic acid

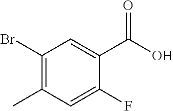

To a solution of 2-fluoro-4-methylbenzoic acid (0.154 g, 1 mmol) in concentrated sulfuric acid (2 mL) was added 1-bromo-2,5-pyrolidinedione (0.178 g, 1 mmol). The resulting mixture was poured into ice-water (10 mL) after stirring for 20 minutes upon which the product precipitated as a white solid. The solid was collected by filtration and then was dissolved in dichloromethane (30 mL) and dried over anhydrous sodium sulfate. The solvent was removed under reduced pressure to afford 5-bromo-2-fluoro-4-methylbenzoic acid as a white solid (0.22 g, 94%).

¹H-NMR (300 MHz, CDCl₃): δ=2.38 (s, 3H), 7.40 (d, 1H), 7.97 (d, 1H), 13.42 (s, 1H).

### Methyl-5-bromo-2-fluoro-4-methylbenzoate

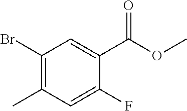

To a solution of 5-bromo-2-fluoro-4-methylbenzoic acid (1.7 g, 7.2 mmol) in thionyl chloride (20 mL) was added DMF (4 drops, cat.). The resulting solution was heated at reflux overnight. The thionyl chloride was removed under reduced pressure and the residue was dissolved in dichloromethane (20 mL). To the solution was added methanol (5 mL) dropwise at 0° C. The reaction was stirred for 30 minutes at room temperature before the solvent was removed under reduced pressure. The residue was purified by silica gel column chromatography eluting with petroleum ether:ethyl acetate=5:1 to afford methyl-5-bromo-2-fluoro-4-methylbenzoate as a brown oil (1.6 g, 89%).

¹H-NMR (300 MHz, DMSO-d₆): δ=2.43 (s, 3H), 3.93 (s, 3H), 7.06 (d, 1H), 8.01 (d, 1H).

### Methyl-2-fluoro-4-methyl-5-vinylbenzoate

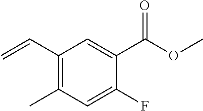

To a solution of methyl-5-bromo-2-fluoro-4-methylbenzoate (0.8 g, 3.23 mmol) in dioxane (20 mL) was added sequentially tributyl(vinyl)stannane (2.05 g, 6.46 mmol), cesium fluoride (0.983 g, 6.46 mmol) and tetrakis(triphenylphosphine)palladium(0) (374 mg, 0.323 mmol) under a argon atmosphere. The resulting suspension was stirred overnight at 100° C. The solvent was removed under reduced pressure. The residue was purified by silica gel column chromatography eluting with petroleum ether: ethyl acetate=10:1 to afford methyl-2-fluoro-4-methyl-5-vinylbenzoate as a thick oil (0.51 g, 81%).

¹H-NMR (300 MHz, CDCl₃): δ=2.38 (s, 3H), 3.94 (s, 3H), 5.33-5.37 (m, 1H), 5.64-5.70 (m, 1H), 6.78-6.95 (m, 2H), 8.01-8.04 (m, 1H).

### 2-Fluoro-4-methyl-5-vinylbenzoic acid

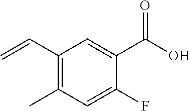

To a solution of methyl-2-fluoro-4-methyl-5-vinylbenzoate (0.51 g, 2.62 mmol) in tetrahydrofuran (10 ml) was added aqueous sodium hydroxide solution (0.315 g in 8 mL water). The reaction was stirred at 60° C. for 2 hours before the organic solvent was removed under reduced pressure. The pH of remaining aqueous layer was adjusted to pH 2 with 2.0 M aqueous hydrochloric acid solution. The aqueous layer was extracted with dichloromethane (3×20 mL). The combined organic layers were dried over anhydrous sodium sulfate and concentrated under reduced pressure to afford 2-fluoro-4-methyl-5-vinylbenzoic acid as a white solid (430 mg, crude), which was used directly without further purification.

### 5-Ethyl-2-fluoro-4-methylbenzoic acid

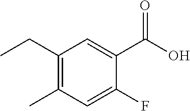

To a solution of 2-fluoro-4-methyl-5-vinylbenzoic acid (0.3 g, 1.6 mmol) was added 5% palladium on carbon (0.1 g). The resulting mixture was stirred for 2 hours under a hydrogen atmosphere. The catalyst was removed by a filtration and the filtrate was concentrated under reduced pressure to afford 5-ethyl-2-fluoro-4-methylbenzoic acid as a white solid (0.27 g, 57% over two steps).

¹H-NMR (300 MHz, CDCl₃): δ=1.24 (m, 3H), 2.36 (s, 3H), 2.60-2.65 (m, 2H), 6.93-6.97 (m, 1H), 7.78-7.80 (m, 1H).

### (R)-Methyl-2-(5-ethyl-2-fluoro-4-methylbenzamido)-3-methylbutanoate

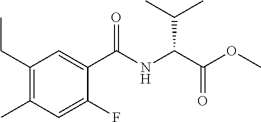 [SMILES: CCc1cc(C(=O)N[C@@H](C(=O)OC)C(C)C)c(F)cc1C]

To a solution of 5-ethyl-2-fluoro-4-methylbenzoic acid (0.2 g, 1.09 mmol) in dichloromethane (10 mL) was added sequentially D-valine hydrochloride (0.22 g, 1.31 mmol), 1-[bis(dimethylamino)methylene]-1H-1,2,3-triazolo[4,5-b]pyridinium-3-oxide hexafluorophosphate (HATU) (0.67 g, 1.6 mmol) and N,N-diisopropylethylamine (0.35 mg, 2.7 mmol). The resulting mixture was stirred for 2 hours at room temperature before the reaction was quenched with ice-water (10 mL). The mixture was extracted with ethyl acetate (3×10 mL) and the combined organic layers were dried over anhydrous sodium sulfate and concentrated under reduced pressure. The residue was purified by silica gel column chromatography eluting with ethyl acetate: ether petroleum=1:10 to afford (R)-methyl-2-(5-ethyl-2-fluoro-4-methylbenzamido)-3-methylbutanoate as a white solid (340 mg, crude), which was used directly without further purification.

LCMS (ESI): m/z=296.2 [M+H]⁺.

### (R)-2-(5-Ethyl-2-fluoro-4-methylbenzamido)-3-methylbutanoic acid

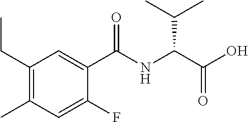 [SMILES: CCc1cc(C(=O)N[C@@H](C(=O)O)C(C)C)c(F)cc1C]

To a solution of (R)-methyl-2-(5-ethyl-2-fluoro-4-methylbenzamido)-3-methylbutanoate (0.34 g, 1.1 mmol) in tetrahydrofuran (10 mL) was added lithium hydroxide (0.083 g in 3 mL water, 3.45 mmol). The resulting suspension was stirred for 2 hours before the organic solvent was removed under reduced pressure. The pH of the remaining aqueous layer was adjusted to pH 3 with 1.0 M aqueous hydrochloric acid solution. The resulting mixture was extracted with ethyl acetate (3×10 mL). The combined organic layers were dried over anhydrous sodium sulfate. The filtrate was concentrated under reduced pressure to afford (R)-2-(5-ethyl-2-fluoro-4-methylbenzamido)-3-methylbutanoic acid as a white solid (0.31 g, 96% over two steps).

### Example 34

### (R)-5-Ethyl-2-fluoro-4-methyl-N-(3-methyl-1-(4-methyl-4-(4-(methylsulfonyl)phenylamino)piperidin-1-yl)-1-oxobutan-2-yl)benzamide

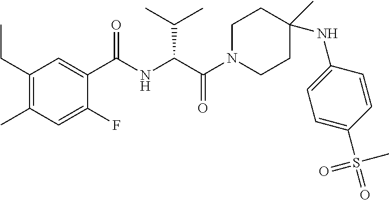 [SMILES: CCc1cc(C(=O)N[C@@H](C(=O)N2CCC(C)(Nc3ccc(S(C)(=O)=O)cc3)CC2)C(C)C)c(F)cc1C]

A solution of tert-butyl-4-methyl-4-(4-(methylsulfonyl)phenylamino)piperidine-1-carboxylate (prepared as described in Example 25-m) (30 mg, 0.081 mmol) in a 6.0 M solution of hydrochloric acid in dioxane (10 mL) was stirred for 2 hours. The solvent was removed under reduced pressure and the residue was dissolved in dichloromethane (10 mL). To the solution was added sequentially (R)-2-(5-ethyl-2-fluoro-4-methylbenzamido)-3-methylbutanoic acid (25 mg, 0.088 mmol), 1-[bis(dimethylamino)methylene]-1H-1,2,3-triazolo[4,5-b]pyridinium-3-oxidehexafluorophosphate (HATU) (46 mg, 0.12 mmol) and N,N-diisopropylethylamine (26 mg, 0.2 mmol). The resulting mixture was stirred for 2 hours at room temperature before the reaction was quenched with ice-water (10 mL). The mixture was extracted with ethyl acetate (3×10 mL) and the combined organic layers were dried over anhydrous sodium sulfate and concentrated under reduced pressure. The residue was purified by prep-HPLC (gradient: 95% water, 5% acetonitrile, 30-50 minutes, gradient to 20% water, 80% acetonitrile) to afford (R)-5-ethyl-2-fluoro-4-methyl-N-(3-methyl-1-(4-methyl-4-(4-(methylsulfonyl)phenylamino)piperidin-1-yl)-1-oxobutan-2-yl)benzamide (Example 34) as a white solid (6.9 mg, 16%).

LCMS (ESI): m/z=532.2 [M+H]⁺.

¹H-NMR (400 MHz, CD₃OD): δ=1.00-1.03 (m, 6H), 1.17-1.26 (m, 3H), 1.48-1.51 (m, 3H), 1.64-1.84 (m, 2H), 2.12-2.28 (m, 3H), 2.37-2.39 (m, 3H), 2.63-2.71 (m, 2H), 3.04 (d, 3H), 3.49-3.69 (m, 2H), 3.72-4.00 (m, 2H), 4.08-4.31 (m, 1H), 6.92-6.95 (m, 2H), 7.04 (dd, 1H), 7.57-7.65 (m, 3H).

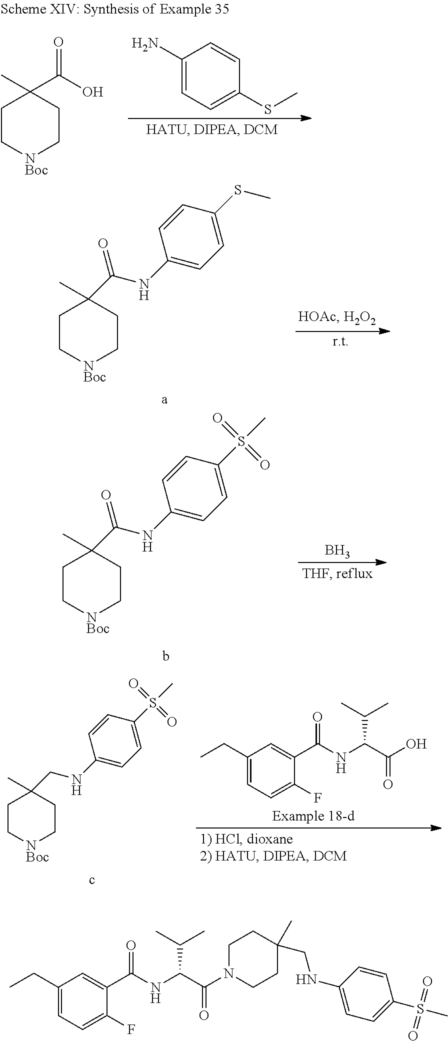

### tert-Butyl-4-methyl-4-(4-(methylthio)phenylcarbamoyl)piperidine-1-carboxylate

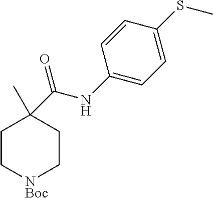 [SMILES: CSc1ccc(NC(=O)C2(C)CCN(C(=O)OC(C)(C)C)CC2)cc1]

To a solution of 1-(tert-butoxycarbonyl)-4-methylpiperidine-4-carboxylic acid (300 mg, 1.23 mmol) in dichloromethane (10 mL) was added sequentially 4-(methylthio)benzenamine (171 mg, 1.23 mmol), 1-[bis(dimethylamino)methylene]-1H-1,2,3-triazolo[4,5-b]pyridinium-3-oxide hexafluorophosphate (HATU) (699 mg, 1.85 mmol) and N,N-diisopropylethylamine (379 mg, 3.08 mmol). The resulting mixture was stirred for 2 hours at room temperature before the reaction was quenched with ice-water (10 mL). The mixture was extracted with dichloromethane (3×10 mL). The combined organic layers were dried over sodium sulfate and concentrated under reduced pressure. The residue was purified by silica gel column chromatography eluting with dichloromethane:methanol=50:1 to afford tert-butyl-4-methyl-4-(4-(methylthio)phenylcarbamoyl)piperidine-1-carboxylate (400 mg, 89%) as a white solid.

LCMS (ESI): m/z=365.2 [M+H]⁺.

### tert-Butyl-4-methyl-4-(4-(methylsulfonyl)phenylcarbamoyl)piperidine-1-carboxylate

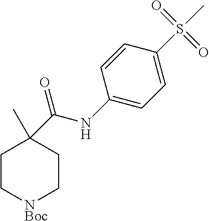 [SMILES: CC(C)(C)OC(=O)N1CCC(C)(C(=O)Nc2ccc(S(C)(=O)=O)cc2)CC1]

To a solution of tert-butyl-4-methyl-4-(4-(methylthio)phenylcarbamoyl)piperidine-1-carboxylate (400 mg, 6.25 mmol) in acetic acid (5 mL) was added a 30% aqueous hydrogen peroxide solution (5 mL, 44 mmol) dropwise at 60° C. The resulting mixture was stirred for 3 additional hours before the solvent was removed under reduced pressure. The residue was purified by gel silica column chromatography eluting with dichloromethane:methanol=200:1 to afford tert-butyl-4-methyl-4-(4-(methylsulfonyl)phenylcarbamoyl)piperidine-1-carboxylate (300 mg, 68%) as a white solid.

LCMS (ESI): m/z=397.2 [M+H]⁺.

¹H-NMR (300 MHz, CD₃OD): δ=1.33 (s, 3H), 1.46-1.54 (m, 11H), 2.14-2.19 (m, 2H), 3.10 (s, 3H), 3.20-3.31 (m, 2H), 3.67-3.74 (m, 2H), 7.88 (s, 4H).

### tert-Butyl-4-methyl-4-((4-(methylsulfonyl)phenylamino)methyl)piperidine-1-carboxylate

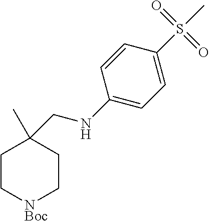 [SMILES: CC1(CNc2ccc(S(C)(=O)=O)cc2)CCN(C(=O)OC(C)(C)C)CC1]

To a solution of tert-butyl-4-methyl-4-(4-(methylsulfonyl)phenylcarbamoyl)piperidine-1-carboxylate (170 mg, 0.428 mmol) in tetrahydrofuran (5 mL) was added a 1.0 M borane-tetrahydrofuran complex solution (0.5 mL, 0.5 mmol) at 0° C. The resulting mixture was the heated at reflux for 6 hours. After cooling to room temperature, to the resulting mixture was added a 1.0 M aqueous hydrochloric acid solution (2 mL) and the mixture was stirred overnight. The reaction mixture was diluted with water (10 mL) and ethyl acetate (30 mL). The organic layer was collected and the aqueous layer was extracted with ethyl acetate (3×30 mL). The combined organic layers were dried over anhydrous sodium sulfate and concentrated under reduced pressure. The residue was purified by silica gel column chromatography eluting with dichloromethane: methanol=100:1 to afford tert-butyl-4-methyl-4-((4-(methylsulfonyl)phenylamino)methyl)piperidine-1-carboxylate (120 mg, 72%) as a brown oil.

LCMS (ESI): m/z=383.2 [M+H]⁺.

¹H-NMR (300 MHz, CDCl₃): δ=0.85-0.94 (m, 2H), 1.06 (s, 3H), 1.2-1.6 (m, 11H), 3.01 (s, 3H), 3.12-3.16 (m, 2H), 3.75-3.78 (m, 2H), 6.64-6.67 (m, 2H), 7.67-7.69 (m, 2H).

### Example 35

### (R)-5-Ethyl-2-fluoro-N-(3-methyl-1-(4-methyl-4-((4-(methylsulfonyl)phenylamino)methyl)piperidin-1-yl)-1-oxobutan-2-yl)benzamide

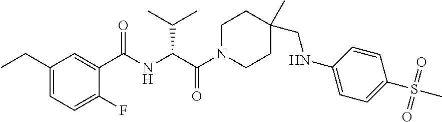 [SMILES: CCc1ccc(F)c(C(=O)N[C@@H](C(=O)N2CCC(C)(CNc3ccc(S(C)(=O)=O)cc3)CC2)C(C)C)c1]

A solution of tert-butyl-4-methyl-4-((4-(methylsulfonyl)phenylamino)methyl)piperidine-1-carboxylate (120 mg, 0.31 mmol) in a 6.0 M solution of hydrochloric acid in dioxane (10 mL) was stirred for 2 hours. The solvent was removed under reduced pressure and the residue was dissolved in dichloromethane (5 mL). To the solution was added sequentially (R)-2-(5-ethyl-2-fluorobenzamido)-3-methylbutanoic acid (prepared as described in Example 18-d) (139 mg, 0.52 mmol), 1-[bis(dimethylamino)methylene]-1H-1,2,3-triazolo[4,5-b]pyridinium-3-oxidehexafluoro phosphate (HATU) (178 mg, 0.47 mmol) and N,N-diisopropylethylamine (100 mg, 0.78 mmol). The resulting mixture was stirred for 2 hours at room temperature before the reaction was quenched with ice-water (10 mL). The mixture was extracted with ethyl acetate (3×10 mL) and the combined organic layers were dried over anhydrous sodium sulfate and concentrated under reduced pressure. The residue was purified by prep-HPLC (gradient: 95% water, 5% acetonitrile, 30-50 minutes, gradient to 20% water, 80% acetonitrile) to afford (R)-5-ethyl-2-fluoro-N-(3-methyl-1-(4-methyl-4-((4-(methylsulfonyl)phenylamino)methyl)piperidin-1-yl)-1-oxobutan-2-yl)benzamide (Example 35) as a white solid (25.1 mg, 42%).

LCMS (ESI): m/z=532.2 [M+H]⁺.

¹H-NMR (400 MHz, CD₃OD): δ=1.02-1.08 (m, 6H), 1.45-1.49 (m, 3H), 1.64-1.87 (m, 2H), 2.12-2.29 (m, 3H), 3.01-3.04 (m, 3H), 3.20-4.19 (m, 4H), 4.99-5.01 (m, 1H), 6.91-6.93 (m, 2H), 7.30-7.49 (m, 4H), 7.60-7.66 (m, 4H), 7.78-7.83 (m, 1H), 7.96-8.01 (m, 1H).

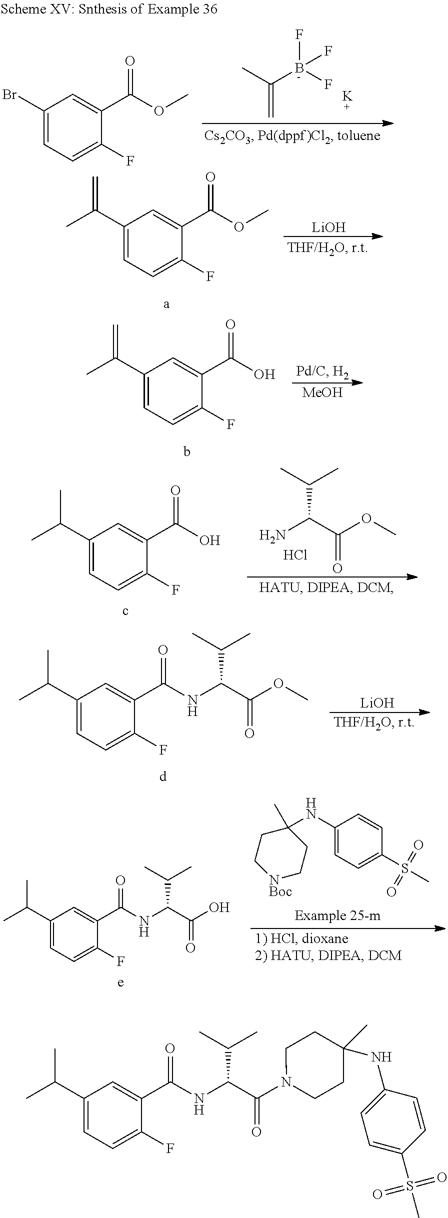

### Methyl-2-fluoro-5-(prop-1-en-2-yl)benzoate

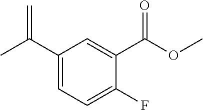

To a solution of methyl-5-bromo-2-fluorobenzoate (100 mg, 0.43 mmol) in toluene (50 mL) was added sequentially potassium trifluoro(prop-1-en-2-yl)borate (64 mg, 0.43 mmol), cesium carbonate (280 mg, 4.3 mmol), [1,1-bis(diphenylphosophino)ferrocene]dichloropalladium(II) (29 mg, 0.04 mmol) and water (5 mL) under an argon atmosphere. The resulting mixture was heated at reflux overnight before the solvent was removed under reduced pressure. The residue was diluted with water (10 mL). The resulting suspension was extracted with ethyl acetate (3×20 mL). The combined organic layers were dried over anhydrous sodium sulfate and concentrated under reduced pressure. The residue was purified by silica gel column chromatography eluting with petroleum ether:ethyl acetate=5:1 to afford methyl-2-fluoro-5-(prop-1-en-2-yl)benzoate (60 mg, 73%) as a yellow oil.

LCMS (ESI): m/z=195.1 [M+H]⁺.

¹H-NMR (300 MHz, DMSO-d₆): δ=2.13 (s, 3H), 3.87 (s, 3H), 5.18 (s, 1H), 5.47 (s, 1H), 7.34 (dd, J=10.7, 8.7 Hz, 1H), 7.81 (ddd, J=8.7, 4.7, 2.6 Hz, 1H), 7.92 (dd, J=7.0, 2.5 Hz, 1H).

### 2-Fluoro-5-(prop-1-en-2-yl)benzoic acid

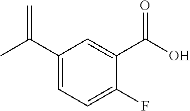

To a solution of methyl-2-fluoro-5-(prop-1-en-2-yl)benzoate (60 mg, 0.31 mmol) in tetrahydrofuran (2 mL) was added 2.0 M aqueous lithium hydroxide solution (2 mL, 20 mmol). The mixture was stirred overnight. The pH of the mixture was adjusted to pH 1 with 1.0 M aqueous hydrochloric acid solution. The resulting suspension was extracted with ethyl acetate (3×30 mL). The combined organic layers were dried over anhydrous sodium sulfate and concentrated under reduced pressure to afford 2-fluoro-5-(prop-1-en-2-yl)benzoic acid (51 mg, crude) as a yellow solid, which was used directly without further purification.

LCMS (ESI): m/z=179.1 [M−H]⁻.

### 2-Fluoro-5-(prop-1-en-2-yl)benzoic acid

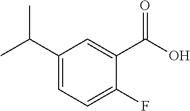

To a solution of 2-fluoro-5-(prop-1-en-2-yl)benzoic acid (600 mg, 3.09 mmol) in methanol (100 mL) was added 5% palladium on carbon (100 mg). The resulting mixture was stirred overnight under a hydrogen atmosphere. The catalyst was removed by filtration and the filtrate was concentrated under reduced pressure. The residue was purified by silica gel column chromatography eluting with dichloromethane:methanol=15:1 to afford 2-fluoro-5-(prop-1-en-2-yl)benzoic acid (300 mg, 50%) as a white solid.

LCMS (ESI): m/z=181.1 [M−H]⁻.

¹H-NMR (300 MHz, CDCl₃): δ=1.28 (d, J=6.9 Hz, 6H), 2.91-2.98 (m, 1H), 7.22-7.01 (m, 1H), 7.43 (dd, J=5.4, 2.8 Hz, 1H), 7.96-7.82 (m, 1H).

### (R)-Methyl-2-(2-fluoro-5-isopropylbenzamido)-3-methylbutanoate

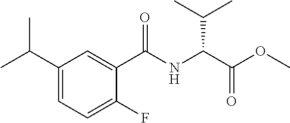 [SMILES: COC(=O)[C@H](NC(=O)c1cc(C(C)C)ccc1F)C(C)C]

To a suspension of 2-fluoro-5-(prop-1-en-2-yl)benzoic acid (150 mg, 0.82 mmol) in dichloromethane (10 mL) was added sequentially (R)-methyl-2-amino-3-methylbutanoate hydrochloride (131 mg, 0.82 mmol), 1-[bis(dimethylamino)methylene]-1H-1,2,3-triazolo[4,5-b]pyridinium-3-oxide hexafluorophosphate (HATU) (466 mg, 1.23 mmol) and N,N-diisopropylethylamine (264 mg, 2.05 mmol). The resulting mixture was stirred for 2 hours at room temperature before the reaction was quenched with ice-water (10 mL). The mixture was extracted with dichloromethane (3×10 mL). The combined organic layers were dried over sodium sulfate and concentrated under reduced pressure. The residue was purified by silica gel column chromatography eluting with petroleum ether: ethyl acetate=10:1 to afford (R)-methyl-2-(2-fluoro-5-isopropylbenzamido)-3-methylbutanoate (130 mg, 53%) as a thick oil.

LCMS (ESI): m/z=296.1 [M+H]⁺.

### (R)-2-(2-Fluoro-5-isopropylbenzamido)-3-methylbutanoic acid

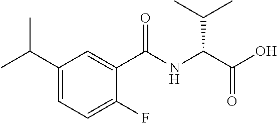 [SMILES: CC(C)c1ccc(F)c(C(=O)N[C@@H](C(=O)O)C(C)C)c1]

To a solution of (R)-methyl-2-(2-fluoro-5-isopropylbenzamido)-3-methylbutanoate (130 mg, 0.44 mmol) in tetrahydrofuran (15 mL) was added a 2.0 M aqueous lithium hydroxide solution (15 mL, 30 mmol). The mixture was stirred overnight. The pH of the mixture was adjusted to pH 1 with 1.0 M aqueous hydrochloric acid solution. The resulting suspension was extracted with ethyl acetate (3×30 mL). The combined organic layers were dried over anhydrous sodium sulfate and concentrated under reduced pressure to afford (R)-2-(2-fluoro-5-isopropylbenzamido)-3-methylbutanoic acid (80 mg, crude) as a yellow solid, which was used directly without further purification.

LCMS (ESI): m/z=281.2 [M+H]⁺.

¹H-NMR (300 MHz, CDCl₃): δ=0.99-1.09 (m, 6H), 1.25-1.29 (m, 6H), 2.38-2.40 (m, 1H), 2.94-2.98 (m, 1H), 4.81-4.85 (m, 1H), 7.05-7.10 (m, 1H), 7.22-7.26 (m, 1H), 7.28-7.35 (m, 1H), 7.94-7.96 (m, 1H).

### Example 36

### (R)-2-Fluoro-5-isopropyl-N-(3-methyl-1-(4-methyl-4-(4-(methylsulfonyl)phenylamino)piperidin-1-yl)-1-oxobutan-2-yl)benzamide

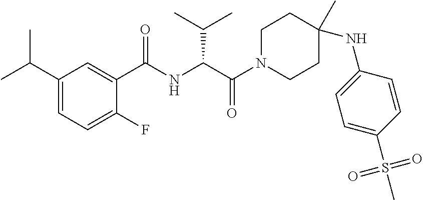 [SMILES: CC(C)c1ccc(F)c(C(=O)N[C@@H](C(=O)N2CCC(C)(Nc3ccc(S(C)(=O)=O)cc3)CC2)C(C)C)c1]

A solution of tert-butyl-4-methyl-4-((4-(methylsulfonyl)phenylamino))piperidine-1-carboxylate (prepared as described in Example 25-m) (120 mg, 0.31 mmol) in a 6.0 M solution of hydrochloric acid in dioxane (10 mL) was stirred for 2 hours. The solvent was removed under reduced pressure and the residue was dissolved in dichloromethane (5 mL). To the solution was added sequentially (R)-2-(2-fluoro-5-isopropylbenzamido)-3-methylbutanoic acid (139 mg, 0.52 mmol), 1-[bis(dimethylamino)methylene]-1H-1,2,3-triazolo[4,5-b]pyridinium-3-oxidehexafluorophosphate (HATU) (178 mg, 0.47 mmol) and N,N-diisopropylethylamine (100 mg, 0.78 mmol). The resulting mixture was stirred for 2 hours at room temperature before the reaction was quenched with ice-water (10 mL). The mixture was extracted with ethyl acetate (3×10 mL) and the combined organic layers were dried over anhydrous sodium sulfate and concentrated under reduced pressure. The residue was purified by prep-HPLC (gradient: 95% water, 5% acetonitrile, 30-50 minutes, gradient to 20% water, 80% acetonitrile) to afford (R)-2-Fluoro-5-isopropyl-N-(3-methyl-1-(4-methyl-4-(4-(methylsulfonyl)phenylamino)piperidin-1-yl)-1-oxobutan-2-yl)benzamide (Example 36) as a white solid (25.1 mg, 42%).

LCMS (ESI): m/z=532.2 [M+H]⁺.

¹H-NMR (400 MHz, CD₃OD): δ=1.02-1.08 (m, 6H), 1.45-1.49 (m, 3H), 1.64-1.87 (m, 2H), 2.12-2.29 (m, 3H), 3.01-3.04 (m, 3H), 3.20-4.19 (m, 4H), 4.99-5.01 (m, 1H), 6.91-6.93 (m, 2H), 7.30-7.49 (m, 4H), 7.60-7.66 (m, 4H), 7.78-7.83 (m, 1H), 7.96-8.01 (m, 1H).

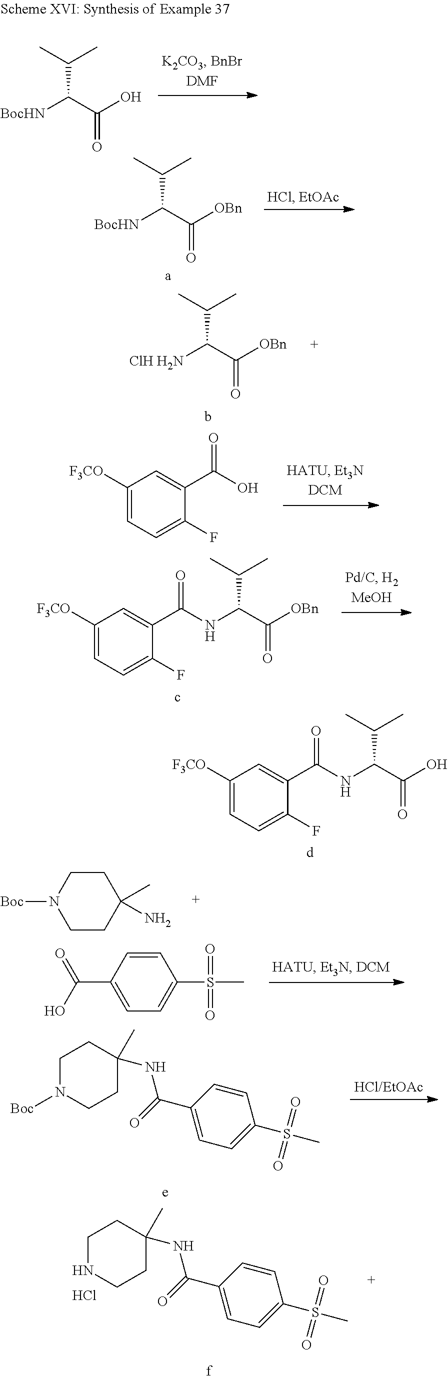

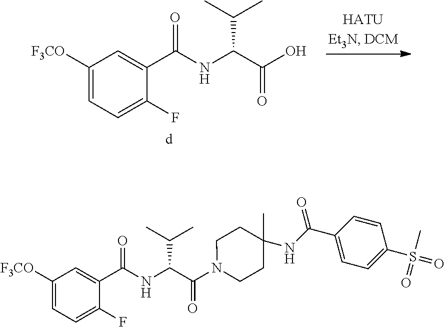 [SMILES: CC(C)[C@@H](NC(=O)c1cc(OC(F)(F)F)ccc1F)C(=O)O ; CC(C)[C@@H](NC(=O)c1cc(OC(F)(F)F)ccc1F)C(=O)N1CCC(C)(NC(=O)c2ccc(S(C)(=O)=O)cc2)CC1]

### (R)-Benzyl-2-(tert-butoxycarbonylamino)-3-methylbutanoate

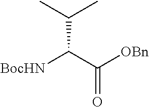 [SMILES: CC(C)[C@@H](NC(=O)OC(C)(C)C)C(=O)OCc1ccccc1]

To a suspension of (R)-2-(tert-butoxycarbonylamino)-3-methylbutanoic acid (300 g, 1.38 mol) and potassium carbonate (380 g, 2.76 mol) in N,N-dimethylformamide (1 L) was added dropwise benzyl bromide (234.6 g, 1.38 mol) slowly at room temperature. The resulting mixture was stirred overnight at room temperature before the reaction was quenched with ice-water (5 L). The resulting suspension was extracted with ethyl acetate (3×1 L). The combined organic layers were dried over anhydrous sodium sulfate and concentrated under reduced pressure. The residue was purified by silica gel column chromatography with ethyl acetate:petroleum ether=1:4 to afford (R)-benzyl-2-(tert-butoxycarbonylamino)-3-methylbutanoate as a thick yellow oil (360 g, 85%).

LCMS (ESI): m/z=308.1 [M+H]⁺.

### (R)-Benzyl-2-amino-3-methylbutanoate hydrochloride

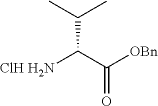

To a solution of (R)-benzyl-2-(tert-butoxycarbonylamino)-3-methylbutanoate (360 g, 1.16 mol) in ethyl acetate (3 L) was bubbled hydrochloric acid (gas) at room temperature until the starting material was consumed. The precipitate was collected by filtration and the solid was dried under reduced pressure to afford (R)-benzyl-2-amino-3-methylbutanoate hydrochloride as a white solid (270 g, 95%).

LCMS (ESI): m/z=208.2 [M+H]⁺.

### (R)-Benzyl-2-(2-fluoro-5-(trifluoromethoxy)benzamido)-3-methylbutanoate

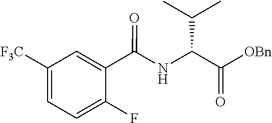 [SMILES: CC(C)[C@@H](NC(=O)c1cc(C(F)(F)F)ccc1F)C(=O)OCc1ccccc1]

To a suspension of 2-fluoro-5-(trifluoromethoxy)benzoic acid (30 g, 134 mmol) in dichloromethane (400 mL) was added (R)-benzyl-2-amino-3-methylbutanoate hydrochloride (34 g, 147 mmol), 1-[bis(dimethylamino)methylene]-1H-1,2,3-triazolo[4,5-b]pyridinium-3-oxide hexafluorophosphate (HATU) (76 g, 201 mmol) and triethylamine (27.1 g, 268 mmol). The resulting mixture was stirred for 2 hours at room temperature before the reaction was quenched with ice-water (500 mL). The mixture was extracted with ethyl acetate (3×300 mL) and the combined organic layers were dried over anhydrous sodium sulfate and concentrated under reduced pressure. The residue was purified by silica gel column chromatography eluting with ethyl acetate:petroleum ether=1:1 to afford (R)-benzyl-2-(2-fluoro-5-(trifluoromethoxy)benzamido)-3-methylbutanoate as a white solid (54 g, 97%).

LCMS (ESI): m/z=414.1 [M+H]⁺.

### (R)-2-(2-Fluoro-5-(trifluoromethoxy)benzamido)-3-methylbutanoic acid

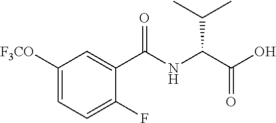 [SMILES: CC(C)[C@@H](NC(=O)c1cc(OC(F)(F)F)ccc1F)C(=O)O]

To a solution of (R)-benzyl-2-(2-fluoro-5-(trifluoromethoxy)benzamido)-3-methylbutanoate (54 g, 131 mmol) in methanol (400 mL) was added 5% palladium on carbon (1 g, dry). The resulting suspension was stirred for 2 hours under a hydrogen atmosphere at room temperature. The mixture was filtered and the filtrate was dried under reduce pressure. The residue was washed with ethyl acetate:petroleum ether=1:4 to afford (R)-2-(2-fluoro-5-(trifluoromethoxy)benzamido)-3-methylbutanoic acid as a white solid (40 g, 95%).

LCMS (ESI): m/z=324.1 [M+H]⁺.

### tert-Butyl-4-methyl-4-(4-(methylsulfonyl)benzamido)piperidine-1-carboxylate

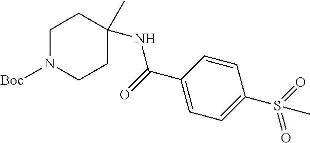 [SMILES: CC1(NC(=O)c2ccc(S(C)(=O)=O)cc2)CCN(C(=O)OC(C)(C)C)CC1]

To a stirred solution of 4-(methylsulfonyl)benzoic acid (200 mg, 1 mmol) in dichloromethane (20 mL) was added tert-butyl 4-amino-4-methylpiperidine-1-carboxylate (214 mg, 1 mmol), 1-[bis(dimethylamino)methylene]-1H-1,2,3-triazolo[4,5-b]pyridinium-3-oxide hexafluorophosphate (HATU) (380 mg, 1 mmol) and triethylamine (0.202 g, 2 mmol). The resulting mixture was stirred for 2 hours at room temperature before the reaction was quenched with water (20 mL). The aqueous layer was extracted with ethyl acetate (2×30 mL) and the combined organic layers were concentrated under reduced pressure. The residue was purified by silica gel column chromatography, eluting with ethyl acetate:petroleum ether=1:1 to afford tert-butyl-4-methyl-4-(4-(methylsulfonyl)benzamido)piperidine-1-carboxylate (0.3 g, 75%) as a colorless oil.

LCMS (ESI): m/z=397.2 [M+H]⁺.

### N-(4-Methylpiperidin-4-yl)-4-(methylsulfonyl)benzamide hydrochloride

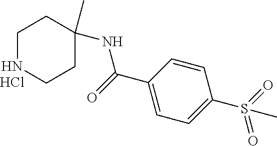 [SMILES: Cl ; CC1(NC(=O)c2ccc(S(C)(=O)=O)cc2)CCNCC1]

Hydrogen chloride (gas) was bubbled into a solution of Tert-butyl 4-methyl-4-(4-(methylsulfonyl)benzamido)piperidine-1-carboxylate (0.3 g, 0.75 mmol) in ethyl acetate (20 mL) at room temperature until the starting material was consumed. The mixture was concentrated under reduced pressure to afford N-(4-methylpiperidin-4-yl)-4-(methylsulfonyl)benzamide hydrochloride as a pale yellow solid (0.26 g, 100%).

LCMS (ESI): m/z=297.1 [M+H]⁺.

### Example 37

### (R)-2-Fluoro-N-(3-methyl-1-(4-methyl-4-(4-(methylsulfonyl)benzamido)piperidin-1-yl)-1-oxobutan-2-yl)-5-(trifluoromethoxy)benzamide

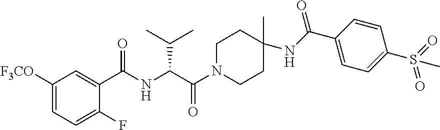 [SMILES: CC(C)[C@@H](NC(=O)c1cc(OC(F)(F)F)ccc1F)C(=O)N1CCC(C)(NC(=O)c2ccc(S(C)(=O)=O)cc2)CC1]

To a mixture of (R)-2-(2-fluoro-5-(trifluoromethoxy)benzamido)-3-methylbutanoic acid (0.16 g, 0.5 mmol) and N-(4-methylpiperidin-4-yl)-4-(methylsulfonyl)benzamide hydrochloride (0.13 g, 0.5 mmol), triethylamine (0.16 g, 1.5 mmol) in dichloromethane (20 mL) was added 1-[bis(dimethylamino)methylene]-1H-1,2,3-triazolo[4,5-b]pyridinium-3-oxide hexafluorophosphate (HATU) (240 mg, 0.63 mmol). The resulting mixture was stirred for 1 hour at room temperature before the reaction was quenched with water (20 mL). The organic layer was concentrated under reduced pressure. The residue was purified by silica gel column chromatography eluting with dichloromethane: methanol=30:1 to afford (R)-2-fluoro-N-(3-methyl-1-(4-methyl-4-(4-(methylsulfonyl)benzamido)piperidin-1-yl)-1-oxobutan-2-yl)-5-(trifluoromethoxy)benzamide (Example 37) as a white solid (65 mg, 22%).

LCMS (ESI): m/z=602.2 [M+H]⁺.

¹H-NMR (400 MHz, CD₃OD): δ=8.44 (t, J=11.9 Hz, 1H), 8.14-8.07 (m, 1H), 8.06-7.97 (m, 4H), 7.66 (s, 1H), 7.52 (dt, J=9.0, 4.7 Hz, 1H), 7.42-7.33 (m, 1H), 4.99 (dd, J=9.7, 5.5 Hz, 1H), 4.23 (d, J=13.9 Hz, 0.5H), 4.10-3.99 (m, 1H), 3.98-3.90 (m, 0.5H), 3.72-3.53 (m, 1H), 3.40 (ddd, J=14.8, 9.0, 3.9 Hz, 0.5H), 3.29-3.21 (m, 0.5H), 3.17 (d, J=4.1 Hz, 3H), 2.53 (d, J=13.1 Hz, 1H), 2.40 (dd, J=16.5, 7.7 Hz, 1H), 2.19 (td, J=13.5, 6.9 Hz, 1H), 1.85-1.72 (m, 1H), 1.71-1.60 (m, 1H), 1.55 (d, J=17.8 Hz, 3H), 1.10-0.96 (m, 6H).

### Example 38

### Prepared Using a Method Analogous to Example 37

### (R)—N-(3-Methyl-1-(4-methyl-4-(4-(methylsulfonyl)benzamido)piperidin-1-yl)-1-oxobutan-2-yl)-3-(trifluoromethoxy)benzamide

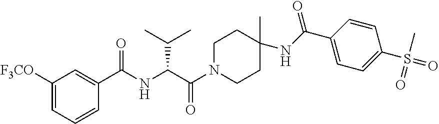 [SMILES: CC(C)[C@@H](NC(=O)c1cccc(OC(F)(F)F)c1)C(=O)N1CCC(C)(NC(=O)c2ccc(S(C)(=O)=O)cc2)CC1]

68.6 mg, yield: 44%, white solid.

LCMS (ESI): m/z=584.2 [M+H]⁺.

¹H-NMR (400 MHz, CD₃OD): δ=8.13-7.96 (m, 4H), 7.88 (t, J=8.6 Hz, 1H), 7.79 (d, J=9.7 Hz, 1H), 7.61 (dd, J=16.6, 8.3 Hz, 1H), 7.50 (t, J=7.8 Hz, 1H), 4.92 (dd, J=8.6, 2.4 Hz, 1H), 4.25-3.99 (m, 2H), 3.72-3.55 (m, 1H), 3.44-3.22 (m, 1H), 3.29-3.21 (m, 1H), 3.17 (d, J=5.8 Hz, 3H), 2.51 (d, J=12.4 Hz, 1H), 2.40 (t, J=10.0 Hz, 1H), 2.24 (br, 1H), 1.82-1.74 (m, 1H), 1.71-1.59 (m, 1H), 1.54 (m, 3H), 1.06 (m, 6H).

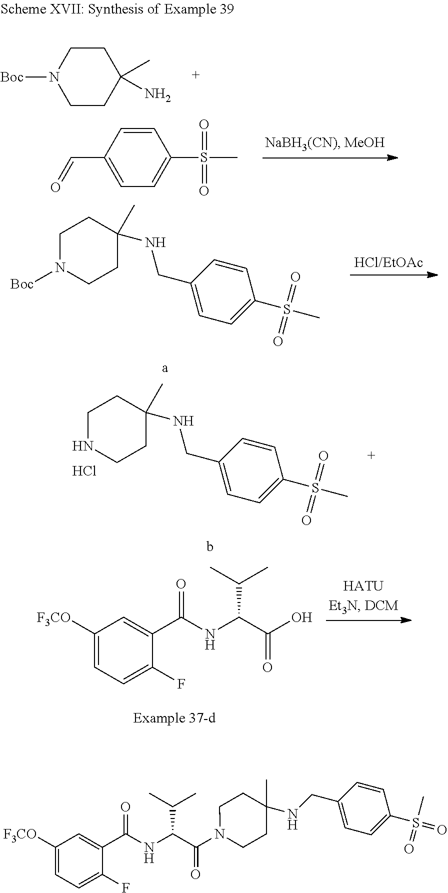

### tert-Butyl-4-methyl-4-((4-(methylsulfonyl)benzyl)amino)piperidine-1-carboxylate

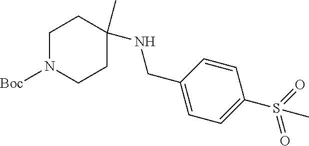 [SMILES: CC1(NCc2ccc(S(C)(=O)=O)cc2)CCN(C(=O)OC(C)(C)C)CC1]

To a stirred solution of 4-(methylsulfonyl)benzaldehyde (550 mg, 3 mmol) and tert-butyl 4-amino-4-methylpiperidine-1-carboxylate (642 mg, 3 mmol) in methanol (20 mL) was added sodium cyanoborohydride (360 mg, 6 mmol). The resulting mixture was stirred for 2 hours at room temperature. The mixture was concentrated under reduced pressure. The residue was purified by silica gel column chromatography, eluting with dichloromethane: methanol=30:1 to afford tert-butyl 4-methyl-4-((4-(methylsulfonyl)benzyl)amino)piperidine-1-carboxylate (0.8 g, 67%) as a pale yellow solid.

LCMS (ESI): m/z=383.2 [M+H]⁺.

### 4-Methyl-N-(4-(methylsulfonyl)benzyl)piperidin-4-amine hydrochloride

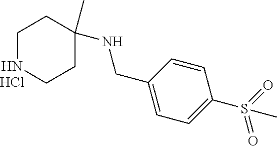 [SMILES: Cl ; CC1(NCc2ccc(S(C)(=O)=O)cc2)CCNCC1]

Hydrogen chloride (gas) was bubbled into a solution of tert-butyl-4-methyl-4-((4-(methylsulfonyl)benzyl)amino)piperidine-1-carboxylate (0.38 g, 1 mmol) in ethyl acetate (20 mL) at room temperature until the starting material was consumed. The mixture was concentrated under reduced pressure to afford 4-methyl-N-(4-(methylsulfonyl)benzyl)piperidin-4-amine hydrochloride (0.32 g, 100%) as a pale yellow solid.

LCMS (ESI): m/z=284.1 [M+H]⁺.

### Example 39

### (R)-2-Fluoro-N-(3-methyl-1-(4-methyl-4-((4-(methylsulfonyl)benzyl)amino)piperidin-1-yl)-1-oxobutan-2-yl)-5-(trifluoromethoxy)benzamide

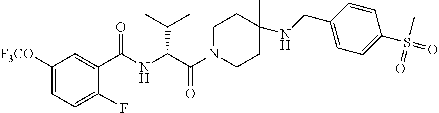 [SMILES: CC(C)[C@@H](NC(=O)c1cc(OC(F)(F)F)ccc1F)C(=O)N1CCC(C)(NCc2ccc(S(C)(=O)=O)cc2)CC1]

To a mixture solution of (R)-2-(2-fluoro-5-(trifluoromethoxy)benzamido)-3-methylbutanoic acid (prepared as described in Example 37-d) (0.16 g, 0.5 mmol) and 4-methyl-N-(4-(methylsulfonyl)benzyl)piperidin-4-amine hydrochloride (0.16 g, 0.5 mmol), triethylamine (0.16 g, 1.5 mmol) in dichloromethane (20 mL) was added 1-[bis(dimethylamino)methylene]-1H-1,2,3-triazolo[4,5-b]pyridinium-3-oxide hexafluorophosphate (HATU) (240 mg, 0.63 mmol). The resulting mixture was stirred for 1 hour at room temperature before the reaction was quenched with water (20 mL). The mixture was extracted with ethyl acetate (3×10 mL) and the combined organic layers were dried over anhydrous sodium sulfate and concentrated under reduced pressure. The residue was purified by silica gel column chromatography eluting with dichloromethane:methanol=30:1 to afford (R)-2-fluoro-N-(3-methyl-1-(4-methyl-4-((4-(methylsulfonyl)benzyl)amino)piperidin-1-yl)-1-oxobutan-2-yl)-5-(trifluoromethoxy)benzamide (Example 39) as a white solid (59 mg, 20%).

LCMS (ESI): m/z=588.2 [M+H]⁺.

¹H-NMR (400 MHz, CD₃OD): δ=7.94 (d, J=7.4 Hz, 2H), 7.74-7.62 (m, 3H), 7.55-7.47 (m, 1H), 7.43-7.33 (m, 1H), 4.98 (dd, J=6.9, 3.6 Hz, 1H), 3.99 (br, 1H), 3.93 (d, J=14.0 Hz, 2H), 3.88-3.44 (m, 3H), 3.13 (d, J=3.4 Hz, 3H), 2.18 (dq, J=13.6, 6.7 Hz, 1H), 1.87-1.56 (m, 4H), 1.33 (d, J=26.2 Hz, 3H), 1.08-0.99 (m, 6H).

### Example 40

### Prepared Using a Method Analogous to Example 39

### (R)-2-Fluoro-N-(1-(4-((4-methoxybenzyl)amino)-4-methylpiperidin-1-yl)-3-methyl-1-oxobutan-2-yl)-5-(trifluoromethoxy)benzamide

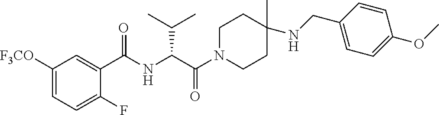 [SMILES: COc1ccc(CNC2(C)CCN(C(=O)[C@H](NC(=O)c3cc(OC(F)(F)F)ccc3F)C(C)C)CC2)cc1]

104 mg, yield 56%, appearance: white solid.

LCMS (ESI): m/z=540.2 [M+H]⁺.

¹H-NMR (400 MHz, CD₃OD): δ=7.65 (d, J=4.5 Hz, 1H), 7.56-7.47 (m, 1H), 7.42-7.32 (m, 3H), 6.98-6.91 (m, 2H), 4.96 (dd, J=12.2, 7.3 Hz, 1H), 4.36-3.97 (m, 2H), 3.89 (d, J=30.3 Hz, 2H), 3.81 (d, J=1.1 Hz, 3H), 3.67-3.13 (m, 2H), 2.22-2.12 (m, 1H), 1.97-1.67 (m, 4H), 1.45 (d, J=35.2 Hz, 3H), 1.09-1.00 (m, 6H).

### Example 41

### Prepared Using a Method Analogous to Example 39

### (R)—N-(3-Methyl-1-(4-methyl-4-((4-(methylsulfonyl)benzyl)amino)piperidin-1-yl)-1-oxobutan-2-yl)-3-(trifluoromethoxy)benzamide

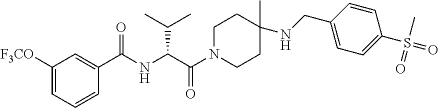 [SMILES: CC(C)[C@@H](NC(=O)c1cccc(OC(F)(F)F)c1)C(=O)N1CCC(C)(NCc2ccc(S(C)(=O)=O)cc2)CC1]

4.5 mg, yield: 4%, appearance: white solid.

LCMS (ESI): m/z=570.2 [M+H]⁺.

¹H-NMR (400 MHz, CD₃OD): δ=8.36 (s, 1H), 8.05 (d, J=8.3 Hz, 2H), 7.89 (d, J=7.8 Hz, 1H), 7.79 (t, J=6.7 Hz, 3H), 7.62 (t, J=8.0 Hz, 1H), 7.51 (d, J=8.3 Hz, 1H), 4.62-4.21 (m, 4H), 3.53 (dt, J=22.8, 11.0 Hz, 1H), 3.36-3.23 (m, 3H), 3.06 (t, J=11.6 Hz, 1H), 2.24 (td, J=13.9, 7.0 Hz, 1H), 2.09-1.91 (m, 4H), 1.88-1.73 (m, 1H), 1.60 (d, J=31.2 Hz, 3H), 1.15-0.93 (m, 6H).

### Example 42

### Prepared Using a Method Analogous to Example 39

### (S)—N-(3-Methyl-1-(4-methyl-4-((4-(methylsulfonyl)benzyl)amino)piperidin-1-yl)-1-oxobutan-2-yl)-3-(trifluoromethoxy)benzamide

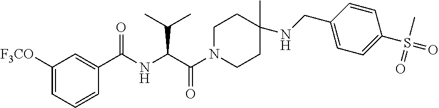 [SMILES: CC(C)[C@H](NC(=O)c1cccc(OC(F)(F)F)c1)C(=O)N1CCC(C)(NCc2ccc(S(C)(=O)=O)cc2)CC1]

4.2 mg, yield: 4%, appearance: white solid.

LCMS (ESI): m/z=570.2 [M+H]⁺.

¹H-NMR (400 MHz, CD₃OD): δ=8.36 (s, 1H), 8.05 (d, J=8.3 Hz, 2H), 7.89 (d, J=7.8 Hz, 1H), 7.79 (t, J=6.7 Hz, 3H), 7.62 (t, J=8.0 Hz, 1H), 7.51 (d, J=8.3 Hz, 1H), 4.62-4.21 (m, 4H), 3.53 (dt, J=22.8, 11.0 Hz, 1H), 3.36-3.23 (m, 3H), 3.06 (t, J=11.6 Hz, 1H), 2.24 (td, J=13.9, 7.0 Hz, 1H), 2.09-1.91 (m, 4H), 1.88-1.73 (m, 1H), 1.60 (d, J=31.2 Hz, 3H), 1.15-0.93 (m, 6H).

### Example 43

### Prepared Using a Method Analogous to Example 39

### (R)—N-(1-(4-((4-Methoxybenzyl)amino)-4-methylpiperidin-1-yl)-3-methyl-1-oxobutan-2-yl)-3-(trifluoromethoxy)benzamide

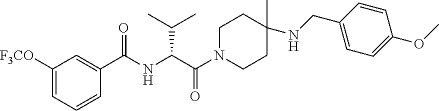 [SMILES: COc1ccc(CNC2(C)CCN(C(=O)[C@H](NC(=O)c3cccc(OC(F)(F)F)c3)C(C)C)CC2)cc1]

41.7 mg, yield: 43%, appearance: white solid.

LCMS (ESI): m/z=522.2 [M+H]⁺.

¹H-NMR (400 MHz, CD₃OD): δ=7.89 (d, J=7.7 Hz, 1H), 7.78 (s, 1H), 7.61 (t, J=8.0 Hz, 1H), 7.50 (d, J=7.7 Hz, 1H), 7.38 (dd, J=8.7, 2.5 Hz, 2H), 6.96 (d, J=7.2 Hz, 2H), 4.34 (dd, J=43.2, 13.9 Hz, 1H), 4.11 (d, J=14.0 Hz, 1H), 3.95 (d, J=27.7 Hz, 2H), 3.81 (s, 3H), 3.55 (dt, J=24.8, 11.6 Hz, 1H), 3.38-3.13 (m, 1H), 2.22 (br, 1H), 2.04-1.66 (m, 4H), 1.48 (d, J=38.6 Hz, 3H), 1.13-0.95 (m, 6H).

Analytical Conditions:

Unless otherwise noted, all solvents, chemicals, and reagents were obtained commercially and used without purification. The ¹H-NMR spectra were obtained in CDCl₃, DMSO-d₆, CD₃OD, or acetone-d₆ at 25° C. at 300 MHz or 400 MHz on an NMR, such as an OXFORD (Varian), with chemical shift (δ, ppm) reported relative to TMS as an internal standard. HPLC-MS chromatograms and spectra were obtained with an HPLC-MS, such as an Agilent 1200-6110 system. Prep-HPLC instruments were on a Prep-HPLC, such as a Gilson GX-281 (Gilson), and P230 Preparative Gradient System (gradient: 95% water, 5% acetonitrile, 30-50 min gradient to 25% water, 75% acetonitrile). The microwave instrument was a microwave reactor, such as a CEM Discover SP.

### Example 44

### Biological Properties of Selected Compounds

### Autotaxin LPC Cascade Assay

### Assay Buffer:

100 mM Tris-HCl, pH=9

### 500 mM NaCl

### 5 mM MgCl₂

### 5 mM CaCl₂

### 0.05% Triton X 100

### Reagents:

| Enzyme | Source | Stock conc. | Working conc. | | Final conc. | |
|---|---|---|---|---|---|---|
| Autotaxin | X-Chem | 4.6 μM | 20 | nM | 5 | nM |
| LPC | Sigma L5254 | 8 mM in | 400 | μM | 100 | μM |
| | | assay buffer | | | | |
| Choline | Sigma C5896 | 50 U/ml in | 0.8 | U/ml | 0.1 | U/ml |
| Oxidase | | water | | | | |
| HRP | Sigma P8375 | 1000 U/ml in | 8 | U/ml | 1 | U/ml |
| | | water | | | | |
| Ampliflu | Sigma 90101 | 100 mM in | 400 | μM | 50 | μM |
| Red | | DMSO | | | | |

### HA130 and Test Compounds:

Highest final concentration is 10 M for HA130 (Echelon, B-0701) and 30 μM for test compounds. Prepare a 1 mM stock of HA130 and a 3 mM stock of each compound in 100% DMSO. Perform a 1:3 serial dilution in DMSO (transfer 5 μl of compound to 10 μl of DMSO). Add 240 μl of buffer to generate a top working concentration of 40 M (HA130) and 120 M (test compound) in 4% DMSO.

Ampliflu Red—Prepare a stock of 5 mM from 100 mM in 100% DMSO. Further dilute to a working concentration of 400 μM in buffer (8% DMSO).

Together with compound this yields a 2% DMSO final concentration.

Choline Oxidase/HRP—0.8 U/ml CO and 8 U/ml HRP.

### Assay Conditions:

Add 5 μl of ATX and 5 μl of compound in a 384 well plate (Corning 3676). Incubate for 10 mins at RT. Add 5 μl of LPC and incubate for 1 hr on the plate shaker. Prepare the CO/HRP and AR prior to use. Add 2.5 μl of CO/HRP and follow with 2.5 μl AR. Note order of addition, CO/HRP mix before AR. Read fluorescence intensity (λex/λem: 530/590 nm) kinetically for 30 min at RT on the Tecan M1000.

TABLE 1
Activity of Selected Compounds
Table 1: IC₅₀ ATX activity values

| IC₅₀ [nM] | Examples |
|---|---|
| ≧10,000 | 17, 22, 24 |
| ≧5,000 ≦ 10,000 | 2, 23 |
| ≧500 ≦ 5,000 | 1, 3, 5, 7, 14, 15, 16, 18, 20, |
| | 29, 30, 37, 38, 42, 43 |
| ≧100 ≦ 500 | 8, 9, 10, 11, 12, 13, 19, 21, 25, |
| | 27, 28, 32, 33, 34, 35, 40, 41 |
| ≧10 ≦ 100 | 4, 6, 26, 31, 36, 39 |

### Further Assays:

An inhibitor of autotaxin is expected to show beneficial effects in human diseases by inhibiting autotaxin in human plasma and tissues as well as in animal models used to recapitulate such human diseases where the disease is caused, mediated and/or propagated by increased LPA levels and/or the activation of ATX. Such diseases which have been reported in the literature include but are not limited to: chronic inflammation, chronic obstructive pulmonary disease (COPD), arthritis, fibrosis, thrombosis, cholestatic pruritus, septic shock, inflammatory bowel disease, asthma, LPS induced lung inflammation, neuropathic pain, atherosclerosis and cardiovascular disease, multiple sclerosis, bone development and cancer. Literature references which describe human diseases where the disease is caused, mediated and/or propagated by increased LPA levels and/or the activation of ATX, autotaxin and LPA and inhibition thereof in in vitro models and animal models used to mimic human diseases caused, mediated and/or propagated by increased LPA levels and/or the activation of ATX include: J Lipid Res. 2014 Mar. 18; 55(7):1192-1214, Cancer Res 2009; 69 (13), 5441; The Journal of Pharmacology and Experimental Therapeutics, 2010, 334 (1), 310; PLOS ONE, 2014, 9 (4), e93230; Biochem. Soc. Trans. 2014, 42, 125; FASEB J. 2014, 28(6), 2655; Arthritis Rheum, 2011, 63(5), 1405; Cell Cycle, 2009, 8 (22), 3695; Mol Carcinog, 2009, 48(9), 801; Clin Cancer Res. 2013 19(23), 6461; Annu Rev Pharmacol Toxicol. 2010, 50, 157; Mol Cancer Ther 208, 7(10), 3352; Front Oncol. 2013, 3, 236; Biomol Ther (Seoul). 2015, 23(1), 1; Osteoarthritis Cartilage 2015, 23(2), 308; Biochim Biophys Acta. 2015, 1851(1), 61; J Lipid Res. 2014, 55(7), 1192; FEBS Lett. 2014, 588(16), 2712; Future Med Chem. 2013, (16), 1935; Biochim Biophys Acta. 2014, 1841(1), 88; Biochim Biophys Acta. 2013, 1831(1), 42; Am J Respir Cell Mol Biol. 2012, 47(5), 563; Am J Respir Cell Mol Biol. 2012, 47(5), 566; Acta Diabetol. 2013, 50(3), 363; Clin Chim Acta. 2012, 413(23-24), 1817; Clin Chim Acta. 2011, 412(13-14), 1201; Life Sci. 2007, 81(12), 1009; Mol Pain 2011, 7, 33; Mol Pain 2010, 6, 78; Biochimie. 2010, 92(6), 698; J Pharmacol Exp Ther. 2010, 333(2), 540; Mol Pain. 2009, 5, 64; Neuroscience 2008, 152(2), 296; Mol Pain 2008, 4, 6; J Neuroimmunol. 2014, 273(1-2), 120; PLoS One. 2013, 8(7), e70941; Nat Rev Rheumatol. 2012, 8(6), 307; J Exp Med. 2012, 209(5), 925; Curr Opin Investig Drugs. 2010, 11(5), 515; Atherosclerosis. 2013, 229(1), 192; J Biol Chem. 2009, 284(11), 7385; Curr Drug Targets 2008, 9(8), 698; Gastroenterology 2010, 139(3), 1008; Lab Invest. 2013, 93(5), 508; J Immunol. 2014, 192(3), 851; Am J Respir Crit Care Med. 2013, 188(8), 889; Am J Respir Crit Care Med. 2013, 188(8), 928; Cell Metab. 2011, 13(5), 592; Exp Cell Res. 2014 Nov. 25. pii: S0014-4827(14)00506-0; Biochem J. 2014, 463(1), 157; Biochim Biophys Acta. 2013, 1831(1), 74; Hepatology 2012, 56(4), 1391; Biochem Soc Trans. 2012, 40(1), 31; Enzyme Res. 2011, 2011:194857. Any of these assays, without limitation, may be used in connection with the invention.

### Abbreviations:

NMR Nuclear magnetic resonance; MDP(S) Mass-directed HPLC purification (system); LC/MS Liquid chromatography mass spectrometry; LDA Lithium diisopropylamide; tert-BuOH tert-Butanol; AcOH Acetic acid; CDI 1,1′-Carbonyldiimidazole; DCE 1,1-Dichloroethane; DCM Dichloromethane; DMF Dimethylformamide; THF Tetrahydrofuran; MeOH Methanol; EtOH Ethanol; EtOAc Ethyl acetate; MeCN Acetonitrile; DMSO Dimethylsulfoxide; Boc tert-Butyloxycarbonyl; DME 1,2-Dimethoxyethane; DMF N,N-Dimethylformamide; DIPEA Diisopropylethylamine; PS-DIEA Polymer-supported diisopropylethylamine; PS-PPh₃-Pd Polymer-supported Pd(PPh₃)₄; LAH Lithium aluminum hydride; EDC 1-(3-Dimethylaminopropyl)-3-ethylcarbodiimide; HATU 1-[bis(dimethylamino)methylene]-1H-1,2,3-triazolo[4,5-b]pyridinium-3-oxide hexafluorophosphate; HOBt 1-Hydroxybenzotnazole; DMAP 4-Dimethylaminopyridine; SEM-Cl 2-(Trimethylsilyl)ethoxymethyl chloride; TBTU O-(Benzotriazol-1-yl)-N,N,N′,N′-tetramethyluronium tetrafluoroborate; TEMPO 2,2,6,6-Tetramethylpiperidine-1-oxyl; TFA (A) Trifluoroacetic acid (anhydride); TLC Thin layer chromatography; TMSCN Trimethylsilyl cyanide; Min Minute(s); NMO N-Methylmorpholine N-oxide; h Hour(s); d Day(s); RT, R.T., r.t., r.t or rt Room temperature; and tR Retention time

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

What is claimed:

1. A compound according to Formula I: 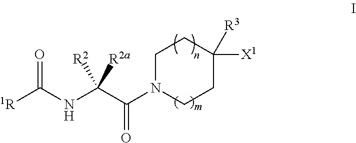
wherein:
X¹ is selected from —C₁₋₂alkylR⁴, —(C₀₋₂alkyl)C(O)R⁴, —(C₀₋₂alkyl)SO₂R⁴, —(C₀₋₂alkyl)NR⁴R^{4a}, —(C₀₋₂alkyl)OR⁴, or —(C₀₋₂alkyl)CR⁴R¹⁰R¹¹;
m and n are each independently selected from 0, 1 or 2;
R¹ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G¹ substituents;
R² is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G² substituents;
R^{2a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G^{2a} substituents;
R² and R^{2a} are each independently a linear structure, or, R² and R^{2a} are taken together with the carbon atom to which they are attached to form a 3-12 membered saturated or unsaturated ring, wherein said ring optionally includes one or more additional heteroatoms selected from O, N, or S(O)ₘ₁;
R³ is selected from —CN, C(O)NR⁷R⁸, S(O)ₙ₀R⁷R⁸, C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, or heteroaryl-C₃₋₁₂heterocycloalkyl-, any of which is optionally substituted with one or more independent G³ substituents;
R⁴ is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G⁴ substituents;
R^{4a} is selected from C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂alkyl-, aryl-C₃₋₁₂cycloalkyl-, aryl-C₃₋₁₂heterocycloalkyl-, heteroaryl-C₀₋₁₂alkyl-, heteroaryl-C₃₋₁₂cycloalkyl-, heteroaryl-C₃₋₁₂heterocycloalkyl-, or pyridine-N-oxide, any of which is optionally substituted with one or more independent G^{4a} substituents;
G¹, G², G^{2a}, G³, G⁴, and G^{4a} are each independently selected from one or more of D, halo, —CN, —CD₃, —OCD₃, -oxo-, —CF₃, —OCF₃, —OCHF₂, —NR⁵R⁶, —NO₂, —B(OH)₂, —PO(OR¹²)₂, —PO(OR¹²)R¹³, —C(O)NR¹²OH, —C₀₋₁₂alkyl, —C₂₋₁₂alkenyl, —C₂₋₁₂alkynyl, C₃₋₁₂cycloalkyl-C₀₋₁₂alkyl-, C₃₋₁₂heterocycloalkyl-C₀₋₁₂alkyl-, aryl-C₀₋₁₂alkyl-, heteroaryl-C₀₋₁₂alkyl-, —OC₀₋₁₂alkyl, —S(O)ₙ₁R¹², —C(O)R¹², —C(O)NR¹²R¹³, —C(O)—C(O)NR¹²R¹³, —C(O)OR¹², —C(O)—C(O)OR¹², —OC(O)R¹², —NR¹²C(O)R¹³, —NR¹²C(O)OR¹³, —NR¹²S(O)₂R¹³, —(CR¹⁴R¹⁵)C(O)R¹³, —(CR¹⁴R¹⁵)C(O)OR¹², —(CR¹⁴R¹⁵)C(O)NR¹²R¹³, —(CR¹⁴R¹⁵)ₙ₁S(O)₂NR¹²R¹³, —(CR¹⁴R¹⁵)ₙ₁NR¹²R¹³, —(CR¹⁴R¹⁵)ₙ₁OR¹², —(CR¹⁴R¹⁵)ₙ₁S(O)ₙ₂R¹², —NR¹⁶C(O)NR¹²R¹³, —NR¹⁶S(O)₂NR¹²R¹³ or —NR¹⁶S(O)NR¹²R¹³, any of which is optionally substituted with one or more independent Q¹ substituents;
Q¹ is selected from H, D, halo, —CN, —CD₃, —OCD₃, -oxo-, —CF₃, —OCF₃, —OCHF₂, —NO₂, —B(OH)₂, —PO(OR¹⁷)₂, —PO(OR¹⁷)R¹⁸, NR¹⁷R¹⁸, —C(O)NR¹⁷OH, C₀₋₁₂alkyl-, —C₂₋₁₂alkenyl, —C₂₋₁₂alkynyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₃₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —C(O)—C(O)NR¹⁷R¹⁸, —C(O)—C(O)OR¹⁷, —OC(O)R¹⁷, —NR¹⁷C(O)R¹⁸, —NR¹⁷S(O)₂R¹⁸, —(CR¹⁹R²⁰)ₙ₃C(O)R¹⁷, —(CR¹⁹R²⁰)ₙ₃C(O)OR¹⁷, —(CR¹⁹R²⁰)ₙ₃C(O)NR¹⁷R¹⁸, —(CR¹⁹R²⁰)ₙ₃S(O)₂NR¹⁷R¹⁸, —(CR¹⁹R²⁰)ₙ₃NR¹⁰R¹⁸, —(CR¹⁹R²⁰)ₙ₃OR¹⁷, —(CR¹⁹R²⁰)ₙ₃S(O)ₙ₄R¹⁷, —NR²¹C(O)NR¹⁷R¹⁸, —NR²¹S(O)₂NR¹⁷R¹⁸ or —NR²¹S(O)NR¹⁷R¹⁸, any of which is optionally substituted with one or more independent Q² substituents;
Q² is selected from one or more of H, D, halo, —CN, -oxo-, —CD₃, —OCD₃, —CF₃, —OCF₃, —OCHF₂, —NO₂, —B(OH)₂, —PO(OR²⁷)₂, —PO(OR²⁷)R²⁸, NR²⁷R²⁸, —C(O)NR²⁷OH, —C₂₋₁₂alkenyl, —C₂₋₁₂alkynyl, —OC₀₋₁₂alkyl, aryl-C₀₋₁₂alkyl-, heteroaryl-C₀₋₁₂alkyl-, C₃₋₁₂cycloalkyl-C₀₋₁₂alkyl-, C₃₋₁₂heterocycloalkyl-C₀₋₁₂alkyl-, aryl-C₀₋₁₂cycloalkyl-, heteroaryl-C₃₋₁₂cycloalkyl-, C₃₋₁₂heterocycloalkyl-C₃₋₁₂cycloalkyl-, C₃₋₁₂cycloalkyl-C₃₋₁₂cycloalkyl-, C₁₋₁₂alkyl-C₃₋₁₂heterocycloalkyl-, C₃₋₁₂heterocycloalkyl-C₃₋₁₂heterocycloalkyl-, aryl-C₃₋₁₂heterocycloalkyl-, heteroaryl-C₃₋₁₂heterocycloalkyl-, —C(O)—C(O)NR²⁷R²⁸, —C₀₋₁₂alkylC(O)OR²⁷, —C(O)—C(O)OR²⁷, —OC(O)R²⁷, —NR²⁷C(O)R²⁸, —NR²⁷C(O)OR²⁸, —NR²⁷S(O)₂R²⁸, —(CR²⁹R³⁰)ₙ₅C(O)R²⁷, —(CR²⁹R³⁰)ₙ₅C(O)OR²⁷, —(CR²⁹R³⁰)ₙ₅C(O)NR²⁷R²⁸, —(CR²⁹R³⁰)ₙ₅S(O)₂NR²⁷R²⁸, —(CR²⁹R³⁰)ₙ₅NR²⁷R²⁸, —(CR²⁹R³⁰)ₙ₅OR²⁷, —(CR²⁹R³⁰)ₙ₅S(O)ₙ₆R²⁷, —NR³⁰C(O)NR²⁷R²⁸, —NR³⁰S(O)₂NR²⁷R²⁸ or —NR³⁰S(O)NR²⁷R²⁸ substituents, any of which may be optionally substituted;
R⁵, R⁶, R⁷, R⁸, R¹⁰, R¹¹, R¹², R¹³, R¹⁴, R¹⁵, and R¹⁶ are each independently selected from one or more of H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
R¹⁷, R¹⁸, R¹⁹, R²⁰, R²¹, R²⁷, R²⁸, R²⁹, and R³⁰ are each independently selected from H, C₁₋₆alkyl-, C₃₋₈cycloalkyl-C₀₋₆alkyl-, C₃₋₈heterocycloalkyl-C₀₋₆alkyl-, aryl-C₀₋₆alkyl-, aryl-C₃₋₈cycloalkyl-, aryl-C₃₋₈heterocycloalkyl-, heteroaryl-C₁₋₆alkyl-, heteroaryl-C₃₋₈cycloalkyl- or heteroaryl-C₃₋₈heterocycloalkyl-, any of which may be optionally substituted;
—NR⁵R⁶ and —NR¹²R¹³ are each independently a linear structure, or, R⁵ and R⁶, or R¹² and R¹³, respectively, are taken together with the nitrogen atom to which they are attached to form a 3-12 membered saturated or unsaturated ring, wherein said ring optionally includes one or more additional heteroatoms selected from O, N, or S(O)ₘ₂;
—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-12 membered saturated or unsaturated ring, wherein said ring optionally includes one or more heteroatoms selected from O, N, or S(O)ₘ₃;
—CR¹⁹R²⁰ is a linear structure, or, R¹⁹ and R²⁰ are taken together with the carbon atom to which they are attached to form a 3-12 membered saturated or unsaturated ring, wherein said ring optionally includes one or more additional heteroatoms selected from O, N, or S(O)ₘ₄;
—NR¹⁷R¹⁸ is a linear structure, or, R¹⁷ and R¹⁸ are taken together with the nitrogen atom to which they are attached to form a 3-12 membered saturated or unsaturated ring, wherein said ring optionally includes one or more additional heteroatoms selected from O, N, or S(O)ₘ₅;
—CR²⁹R³⁰ is a linear structure, or, R²⁹ and R³⁰ are taken together with the carbon atom to which they are attached to form a 3-12 membered saturated or unsaturated ring, wherein said ring optionally includes one or more additional heteroatoms selected from O, N, or S(O)ₘ₆;
—NR²⁷R²⁸ is a linear structure, or, R²⁷ and R²⁸ are taken together with the nitrogen atom to which they are attached to form a 3-12 membered saturated or unsaturated ring, wherein said ring optionally includes one or more additional heteroatoms selected from O, N, or S(O)ₘ₇;
wherein m1, m2, m3, m4, m5, m6, m7, n0, n1, n2, n3, n4, n5 and n6 are each independently selected from 0, 1 or 2;
or a pharmaceutically acceptable salt, solvate or a prodrug thereof.

2. The compound or salt of any one of the preceding claims, wherein:
R¹ is selected from C₀₋₈alkyl-, C₃₋₈cycloalkyl-C₀₋₈alkyl-, or aryl-C₀₋₈alkyl-;
G¹ is selected from one or more of D, halo, —CN, —CD₃, —OCD₃, -oxo-, —CF₃, —OCF₃, —OCHF₂, —B(OH)₂, —C₀₋₈alkyl, —C₂₋₈alkenyl, —C₂₋₈alkynyl, C₃₋₈cycloalkyl-C₀₋₈alkyl-, C₃₋₈heterocycloalkyl-C₀₋₈alkyl-, aryl-C₀₋₈alkyl-, heteroaryl-C₀₋₈alkyl-, —OC₀₋₈alkyl, or —S(O)ₙ₁R¹².

3. The compound or salt of any one of the preceding claims, wherein G¹ is selected from 0 to 3 of D, halo, —CN, —CD₃, —OCD₃, -oxo-, —CF₃, —OCF₃, —OCHF₂, —B(OH)₂, —C₀₋₈alkyl, —C₂₋₈alkenyl, —C₂₋₈alkynyl, C₃₋₈cycloalkyl-C₀₋₈alkyl-, C₃₋₈heterocycloalkyl-C₀₋₈alkyl-, aryl-C₀₋₈alkyl-, heteroaryl-C₀₋₈alkyl-, —OC₀₋₈alkyl, or —S(O)ₙ₁R¹².

4. The compound or salt of any one of the preceding claims, wherein:
R¹ is selected from C₀₋₂alkyl-, C₄₋₆cycloalkyl-C₀₋₂alkyl-, or aryl-C₀₋₂alkyl-;
G¹ is selected from one or more of D, halo, —CN, —CD₃, —OCD₃, -oxo-, —CF₃, —OCF₃, —OCHF₂, —B(OH)₂, —C₀₋₂alkyl, —C₂₋₄alkenyl, —C₂₋₄alkynyl, C₄₋₆cycloalkyl-C₀₋₂alkyl-, C₄₋₆heterocycloalkyl-C₀₋₂alkyl-, aryl-C₀₋₃alkyl-, heteroaryl-C₀₋₂alkyl-, —OC₀₋₂alkyl, or —S(O)ₙ₁R¹².

5. The compound or salt of any one of the preceding claims, wherein G¹ is selected from is selected from 0 to 2 of D, halo, —CN, —CD₃, —OCD₃, -oxo-, —CF₃, —OCF₃, —OCHF₂, —B(OH)₂, —C₀₋₂alkyl, —C₂₋₄alkenyl, —C₂₋₄alkynyl, C₄₋₆cycloalkyl-C₀₋₂alkyl-, C₄₋₆heterocycloalkyl-C₀₋₂alkyl-, aryl-C₀₋₃alkyl-heteroaryl-C₀₋₂alkyl-, —OC₀₋₂alkyl, or —S(O)ₙ₁R¹².

6. The compound or salt of any one of the preceding claims, wherein:
R² is selected from C₀₋₈alkyl-, C₃₋₈cycloalkyl-C₀₋₈alkyl-, or C₃₋₈heterocycloalkyl-C₀₋₈alkyl-;
R^{2a} is C₀₋₈alkyl-; or
R² and R^{2a} are each independently a linear structure, or, R² and R^{2a} are taken together with the carbon atom to which they are attached to form a 3-6 membered saturated or unsaturated ring;
G² and G^{2a} are each independently selected from one or more of D, halo, —CN, —CD₃, —OCD₃, -oxo-, —CF₃, —OCF₃, —OCHF₂, —C₀₋₈alkyl, —C₂₋₈alkenyl, —C₂₋₈alkynyl, C₃₋₈cycloalkyl-C₀₋₈alkyl-, or —OC₀₋₈alkyl.

7. The compound or salt of any one of the preceding claims, wherein G² and G^{2a} are each independently selected from 0 to 3 of D, halo, —CN, —CD₃, —OCD₃, -oxo-, —CF₃, —OCF₃, —OCHF₂, —C₀₋₈alkyl, —C₂₋₈alkenyl, —C₂₋₈alkynyl, C₃₋₈cycloalkyl-C₀₋₈alkyl-, or —OC₀₋₈alkyl.

8. The compound or salt of any one of the preceding claims, wherein:
R² is selected from C₀₋₂alkyl-, C₄₋₆cycloalkyl-C₀₋₂alkyl-, or C₄₋₆heterocycloalkyl-C₀₋₂alkyl-;
R^{2a} is C₀₋₂alkyl-; or
R² and R^{2a} are each independently a linear structure, or, R² and R^{2a} are taken together with the carbon atom to which they are attached to form a 3-6 membered saturated or unsaturated ring;
G² and G^{2a} are each independently selected from one or more of D, halo, —CN, —CD₃, —OCD₃, -oxo-, —CF₃, —OCF₃, —OCHF₂, —C₀₋₂alkyl, —C₂₋₄alkenyl, —C₂₋₄alkynyl, C₄₋₆cycloalkyl-C₀₋₂alkyl-, or —OC₀₋₂alkyl.

9. The compound or salt of any one of the preceding claims, wherein G² and G^{2a} are each independently selected from 0 to 2 of D, halo, —CN, —CD₃, —OCD₃, -oxo-, —CF₃, —OCF₃, —OCHF₂, —C₀₋₂alkyl, —C₂₋₄alkenyl, —C₂₋₄alkynyl, C₄₋₆cycloalkyl-C₀₋₂alkyl-, or —OC₀₋₂alkyl.

10. The compound or salt of any one of the preceding claims, wherein R³ is selected from —CN, C(O)NR⁷R⁸, S(O)ₙ₀R⁷R⁸, C₀₋₈alkyl, or C₃₋₈cycloalkyl-C₀₋₈alkyl-.

11. The compound or salt of any one of the preceding claims, wherein R³ is selected from —CN, C(O)NR⁷R⁸, S(O)ₙ₀R⁷R⁸, C₀₋₂alkyl, or C₄₋₆cycloalkyl-C₀₋₂alkyl-.

12. The compound or salt of any one of the preceding claims, wherein:
R⁴ is selected from C₀₋₈alkyl-, C₃₋₈cycloalkyl-C₀₋₈alkyl-, C₃₋₈heterocycloalkyl-C₀₋₈alkyl-, aryl-C₀₋₈alkyl-, aryl-C₃₋₈cycloalkyl-, aryl-C₃₋₈-heterocycloalkyl-, heteroaryl-C₀₋₈alkyl-, heteroaryl-C₃₋₈cycloalkyl-, heteroaryl-C₃₋₈-heterocycloalkyl-, or pyridine-N-oxide;
R^{4a} is selected from C₀₋₈alkyl-, C₃₋₈cycloalkyl-C₀₋₈alkyl-, aryl-C₀₋₈alkyl-;
G⁴ is selected from one or more of D, halo, —CN, —CD₃, —OCD₃, -oxo-, —CF₃, —OCF₃, —OCHF₂, —NR⁵R⁶, —C(O)NR¹²OH, —C₀₋₈alkyl, —C₂₋₈alkenyl, —C₂₋₈alkynyl, —OC₀₋₈alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

13. The compound or salt of any one of the preceding claims, wherein:
G⁴ is selected from 0 to 3 of D, halo, —CN, —CD₃, —OCD₃, -oxo-, —CF₃, —OCF₃, —OCHF₂, —NR⁵R⁶, —C(O)NR¹²OH, —C₀₋₈alkyl, —C₂₋₈alkenyl, —C₂₋₈alkynyl, —OC₀₋₈alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹ 2S(O)₂R¹³, —(CR¹⁴R¹⁵)ₙ₁OR¹², or —(CR¹⁴R¹⁵)ₙ₁S(O)ₙ₂R¹².

14. The compound or salt of any one of the preceding claims, wherein:
R⁴ is selected from C₀₋₂alkyl-, C₄₋₆cycloalkyl-C₀₋₂alkyl-, C₄₋₆heterocycloalkyl-C₀₋₂alkyl-, aryl-C₀₋₂alkyl-, aryl-C₄₋₆cycloalkyl-, aryl-C₄₋₆heterocycloalkyl-, heteroaryl-C₀₋₂alkyl-, heteroaryl-C₄₋₆cycloalkyl-, heteroaryl-C₄₋₆heterocycloalkyl-, or pyridine-N-oxide;
R^{4a} is selected from C₀₋₂alkyl-, C₄₋₆cycloalkyl-C₀₋₂alkyl-, aryl-C₀₋₂alkyl-;
G⁴ is selected from one or more of D, halo, —CN, —CD₃, —OCD₃, -oxo-, —CF₃, —OCF₃, —OCHF₂, —NR⁵R⁶, —C(O)NR¹²OH, —C₀₋₂alkyl, —C₂₋₄alkenyl, —C₂₋₄alkynyl, —OC₀₋₂alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

15. The compound or salt of any one of the preceding claims, wherein:
G⁴ is selected from 0 to 2 of D, halo, —CN, —CD₃, —OCD₃, -oxo-, —CF₃, —OCF₃, —OCHF₂, —NR⁵R⁶, —C(O)NR¹²OH, —C₀₋₂alkyl, —C₂₋₄alkenyl, —C₂₋₄alkynyl, —OC₀₋₂alkyl, —S(O)ₙ₁R¹², —C(O)R¹², —C(O)NR¹²R¹³, —C(O)OR¹², —NR¹²C(O)R¹³, —NR¹²C(O)OR¹³, —NR¹²S(O)₂R¹³, —(CR¹⁴R¹⁵)ₙ₁OR¹², or —(CR¹⁴R¹⁵)ₙ₁S(O)ₙ₂R¹².

16. The compound or salt of any one of the preceding claims, wherein:
R² is selected from methyl, ethyl, propyl, isopropyl, or one of the following groups: 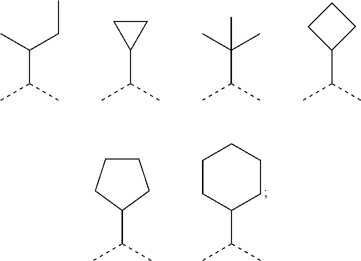
and R^{2a} is selected from H, methyl, ethyl, propyl, or isopropyl; or
R² and R^{2a} are taken together with the carbon atom to which they are attached to form one of the following groups:

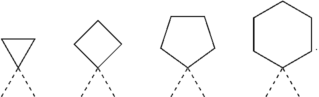

17. The compound or salt of any one of the preceding claims, wherein:
—NR⁵R⁶ and —NR¹²R¹³ are each independently a linear structure, or, R⁵ and R⁶, or R¹² and R¹³, respectively, are taken together with the nitrogen atom to which they are attached to form a 3-6 membered saturated or unsaturated ring, wherein said ring optionally includes one or more additional heteroatoms selected from O, N, or S(O)ₘ₂;
wherein m2 is selected from 0, 1 or 2.

18. The compound or salt of any one of the preceding claims, wherein:
—CR¹⁰R¹¹ and —CR¹⁴R¹⁵ are each independently a linear structure, or, R¹⁰ and R¹¹, or R¹⁴ and R¹⁵ respectively, are taken together with the carbon atom to which they are attached to form a 3-6 membered saturated or unsaturated ring, wherein said ring optionally includes one or more heteroatoms selected from O, N, or S(O)ₘ₃;
wherein m3 is selected from 0, 1 or 2.

19. The compound or salt of any one of the preceding claims, wherein:
—CR¹⁹R²⁰ is a linear structure, or, R¹⁹ and R²⁰ are taken together with the carbon atom to which they are attached to form a 3-6 membered saturated or unsaturated ring, wherein said ring optionally includes one or more additional heteroatoms selected from O, N, or S(O)ₘ₄;
wherein m4 is selected from 0, 1 or 2.

20. The compound or salt of any one of the preceding claims, wherein:
—NR¹⁷R¹⁸ is a linear structure, or, R¹⁷ and R¹⁸ are taken together with the nitrogen atom to which they are attached to form a 3-6 membered saturated or unsaturated ring, wherein said ring optionally includes one or more additional heteroatoms selected from O, N, or S(O)ₘ₅;
wherein m5 is selected from 0, 1 or 2.

21. The compound or salt of any one of the preceding claims, wherein:
—CR²⁹R³⁰ is a linear structure, or, R²⁹ and R³⁰ are taken together with the carbon atom to which they are attached to form a 3-6 membered saturated or unsaturated ring, wherein said ring optionally includes one or more additional heteroatoms selected from O, N, or S(O)ₘ₆;
wherein m6 is selected from 0, 1 or 2.

22. The compound or salt of any one of the preceding claims, wherein:
—NR²⁷R²⁸ is a linear structure, or, R²⁷ and R²⁸ are taken together with the nitrogen atom to which they are attached to form a 3-6 membered saturated or unsaturated ring, wherein said ring optionally includes one or more additional heteroatoms selected from O, N, or S(O)ₘ₇;
wherein m7 is selected from 0, 1 or 2.

23. The compound or salt of any one of the preceding claims, wherein:
R¹ is selected from one of C₆cycloalkyl-C₀₋₆alkyl-, C₆heterocycloalkyl-C₀₋₆alkyl-, 6-membered-aryl-C₀₋₆alkyl-, or 6-membered-heteroaryl-C₀₋₆alkyl-,
wherein the 4-position of R¹ is hydrogen, and wherein R¹ is optionally substituted by one or more G¹ substituents at the 2, 3, 5 and 6 positions.

24. The compound or salt of any one of the preceding claims, which is represented by the Formula Ia:

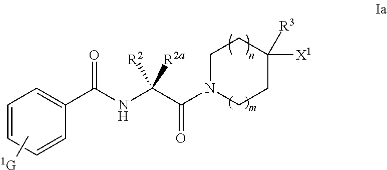

25. The compound of claim 1, wherein said compound has the structure of Formula Id:

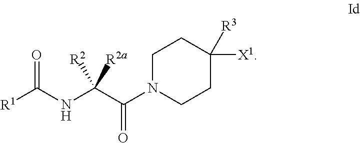

26. The compound of claim 25, wherein R¹ is aryl substituted with one or more independent G¹ substituents.

27. The compound of claim 26, wherein said G¹ substituents are each, independently, hydrogen, halo, C₁₋₁₂alkyl, CF₃, OCF₃, OCHF₂, aryl-C₁₋₁₂alkyl, aryl, C₃₋₁₂cycloalkyl, or two G¹ substituents combine to form, with the carbons to which they are attached, an optionally substituted C₃₋₁₂cycloalkyl.

28. The compound of any one of claims 25 to 27, wherein R¹ is 2-fluoro-3-methyl-phenyl, 2-fluoro-5-ethyl-phenyl, 2-fluoro-5-methoxy-phenyl, 2-fluoro-5-trifluoromethoxy-phenyl, 3-trifluoromethoxy-phenyl, 3-methyl-phenyl, 2-fluoro-5-trifluoromethyl-phenyl, 6-fluoro-3-methyl-2,3-dihydro-1H-indene, 2-fluoro-5-difluoromethyl-phenyl, 2-fluoro-5-tert-butyl-phenyl, 2-fluoro-5-benzyl-phenyl, 2-fluoro-5-sec-butyl-phenyl, 2-fluoro-5-phenyl-phenyl, 2-fluoro-5-cyclopropyl-phenyl, 2-fluoro-4-methyl-5-ethyl-phenyl, or 2-fluoro-5-iso-propyl-phenyl.

29. The compound of any one of claims 25 to 28, wherein R² is hydrogen, C₁₋₁₂alkyl, or C₃₋₁₂cycloalkyl.

30. The compound of claim 29, wherein R² is hydrogen, iso-propyl, or cyclopropyl.

31. The compound of any one of claims 25 to 30, wherein R^{2a} is hydrogen or C₁₋₁₂alkyl.

32. The compound of claim 31, wherein R^{2a} is hydrogen or iso-propyl.

33. The compound of any one of claims 30 to 32, wherein if one of R² or R^{2a} is C₁₋₁₂alkyl, or C₃₋₁₂cycloalkyl, the other is hydrogen.

34. The compound of any one of claims 25 to 33, wherein R³ is hydrogen, CN, C(O)NR⁷R⁸, or C₁₋₁₂alkyl.

35. The compound of claim 34, wherein R³ is hydrogen, —CN, —C(O)NH(CH₃), —C(O)N(CH₃)₂, methyl, or —CH₂OCH₃.

36. The compound of any one of claims 25 to 35, wherein X¹ is —(C₀₋₂alkyl)-NR⁴R^{4a} or —(C₀₋₂alkyl)-OR⁴.

37. The compound of claim 36, wherein R^{4a} is hydrogen or methyl.

38. The compound of claim 36 or 37, wherein R⁴ is aryl, aryl-C₁₋₁₂alkyl, or heteroaryl substituted with one or more independent G⁴ substituents.

39. The compound of claim 38, wherein said G⁴ substituents are hydrogen, —CN, —OC₀₋₁₂alkyl, —NR¹²C(O)R¹³, —C(O)OR¹², or —C₀₋₁₂alkyl-S(O)ₙ₁R¹².

40. The compound of claim 39, wherein said G⁴ substituents are hydrogen, —CN, —OCH₃, —NHC(O)CH₃, —CH₂—SO₂CH₃, —CH₂—SO₂CH₃, —C(O)OH, or —C(O)OtBu.

41. The compound of any one of claims 25 to 40, wherein X¹ is:

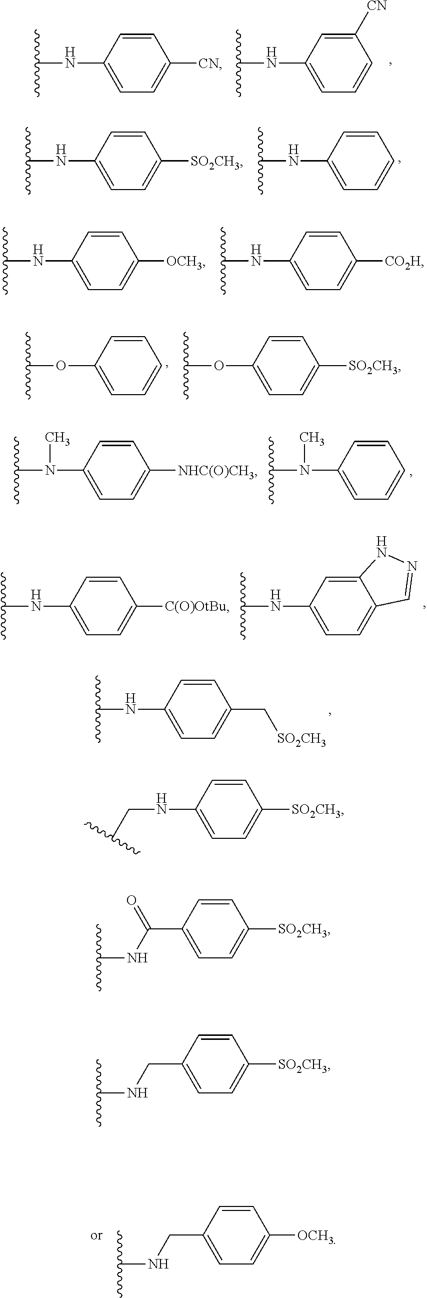

42. The compound or salt of any one of the preceding claims, wherein m and n are each equal to 1.

43. The compound or salt of any one of the preceding claims, wherein X¹ is selected from C₁₋₂alkylR⁴, —(C₀₋₁alkyl)NR⁴R^{4a}, or —(C₀₋₁alkyl)OR⁴.

44. A compound selected from:
(R)—N-(1-(4-(4-Cyanophenylamino)-4-methylpiperidin-1-yl)-3-methyl-1-oxobutan-2-yl)-2-fluoro-3-methylbenzamide;
(R)—N-(1-(4-(3-Cyanophenylamino)-4-methylpiperidin-1-yl)-3-methyl-1-oxobutan-2-yl)-2-fluoro-3-methylbenzamide;
(R)-2-Fluoro-3-methyl-N-(3-methyl-1-(4-methyl-4-(4-(methylsulfonyl)phenylamino)piperidin-1-yl)-1-oxobutan-2-yl)benzamide;
(R)-5-Ethyl-2-fluoro-N-(3-methyl-1-(4-methyl-4-(4-(methylsulfonyl)phenylamino)piperidin-1-yl)-1-oxobutan-2-yl)benzamide;
(R)-2-Fluoro-5-methoxy-N-(3-methyl-1-(4-methyl-4-(4-(methylsulfonyl)phenylamino)piperidin-1-yl)-1-oxobutan-2-yl)benzamide;
(R)-2-Fluoro-N-(3-methyl-1-(4-methyl-4-(4-(methylsulfonyl)phenylamino)piperidin-1-yl)-1-oxobutan-2-yl)-5-(trifluoromethoxy)benzamide;
(R)—N-(3-Methyl-1-(4-methyl-4-(phenylamino)piperidin-1-yl)-1-oxobutan-2-yl)-3-(trifluoromethoxy) benzamide;
(R)—N-(1-(4-((4-Methoxyphenyl)amino)-4-methylpiperidin-1-yl)-3-methyl-1-oxobutan-2-yl)-3-(trifluoromethoxy)benzamide;
(R)-4-((4-Methyl-1-(3-methyl-2-(3-(trifluoromethoxy)benzamido)butanoyl)piperidin-4-yl)amino)benzoic acid;
(R)-2-Fluoro-N-(3-methyl-1-(4-methyl-4-(phenylamino)piperidin-1-yl)-1-oxobutan-2-yl)-5-(trifluoromethoxy)benzamide;
(R)-4-((1-(2-(2-Fluoro-5-(trifluoromethoxy)benzamido)-3-methylbutanoyl)-4-methylpiperidin-4-yl)amino)benzoic acid;
(R)-2-Fluoro-N-(1-(4-((4-methoxyphenyl)amino)-4-methylpiperidin-1-yl)-3-methyl-1-oxobutan-2-yl)-5-(trifluoromethoxy)benzamide;
(R)—N-(1-Cyclopropyl-2-(4-methyl-4-((4-(methylsulfonyl)phenyl)amino)piperidin-1-yl)-2-oxoethyl)-2-fluoro-5-(trifluoromethoxy)benzamide;
(R)-1-(2-(2-Fluoro-3-methylbenzamido)-3-methylbutanoyl)-N-methyl-4-phenoxypiperidine-4-carboxamide;
(R)-1-(2-(5-Ethyl-2-fluorobenzamido)-3-methylbutanoyl)-N-methyl-4-(4-(methylsulfonyl)phenoxy)piperidine-4-carboxamide;
(R)-1-(2-(5-Ethyl-2-fluorobenzamido)-3-methylbutanoyl)-N,N-dimethyl-4-(4-(methylsulfonyl)phenoxy)piperidine-4-carboxamide;
(R)—N-(1-(4-(Methoxymethyl)-4-(phenylamino)piperidin-1-yl)-3-methyl-1-oxobutan-2-yl)-3-methylbenzamide;
(R)—N-(1-(4-Cyano-4-(4-(methylsulfonyl)phenylamino)piperidin-1-yl)-3-methyl-1-oxobutan-2-yl)-5-ethyl-2-fluorobenzamide;
(R)—N-(1-(4-(4-Acetamidophenyl(methyl)amino)-4-methylpiperidin-1-yl)-3-methyl-1-oxobutan-2-yl)-2-fluoro-5-(trifluoromethyl)benzamide;
(R)-3-Methyl-N-(3-methyl-1-(4-methyl-4-(methyl(phenyl)amino)piperidin-1-yl)-1-oxobutan-2-yl)benzamide;
(R)-4-(1-(2-(2-Fluoro-5-(trifluoromethyl)benzamido)-3-methylbutanoyl)-4-methylpiperidin-4-ylamino)benzoic acid;
(R)-2-Fluoro-N-(3-methyl-1-(4-(4-(methylsulfonyl)phenylamino)piperidin-1-yl)-1-oxobutan-2-yl)-5-(trifluoromethyl)benzamide;
(R)—N-(1-(4-(1H-Indazol-5-ylamino)piperidin-1-yl)-3-methyl-1-oxobutan-2-yl)-2-fluoro-5-(trifluoromethyl)benzamide;
(R)-4-(1-(2-(2-Fluoro-5-(trifluoromethyl)benzamido)-3-methylbutanoyl)piperidin-4-ylamino)benzoic acid;
6-Fluoro-3-methyl-N—((R)-3-methyl-1-(4-methyl-4-(4-(methylsulfonyl) phenylamino)piperidin-1-yl)-1-oxobutan-2-yl)-2,3-dihydro-1H-indene-5-carboxamide;
(R)-5-Ethyl-2-fluoro-N-(3-methyl-1-(4-methyl-4-(4-(methylsulfonylmethyl)phenylamino)piperidin-1-yl)-1-oxobutan-2-yl)benzamide;
(R)-2-Fluoro-N-(3-methyl-1-(4-methyl-4-(4-(methylsulfonyl)phenylamino)piperidin-1-yl)-1-oxobutan-2-yl)-5-(trifluoromethyl)benzamide;
(R)-5-(Difluoromethoxy)-2-fluoro-N-(3-methyl-1-(4-methyl-4-(4-(methyl sulfonyl)phenylamino)piperidin-1-yl)-1-oxobutan-2-yl)benzamide;
(R)-5-tert-Butyl-2-fluoro-N-(3-methyl-1-(4-methyl-4-(4-(methylsulfonyl) phenylamino)piperidin-1-yl)-1-oxobutan-2-yl)benzamide;
(R)-5-Benzyl-2-fluoro-N-(3-methyl-1-(4-methyl-4-(4-(methylsulfonyl)phenyl amino)piperidin-1-yl)-1-oxobutan-2-yl)benzamide;
(R)-2-Fluoro-5-isobutyl-N-(3-methyl-1-(4-methyl-4-(4-(methylsulfonyl)phenylamino)piperidin-1-yl)-1-oxobutan-2-yl)benzamide;
(R)-4-Fluoro-N-(3-methyl-1-(4-methyl-4-(4-(methylsulfonyl)phenylamino)piperidin-1-yl)-1-oxobutan-2-yl)biphenyl-3-carboxamide;
(R)-5-Cyclopropyl-2-fluoro-N-(3-methyl-1-(4-methyl-4-(4-(methylsulfonyl)phenylamino)piperidin-1-yl)-1-oxobutan-2-yl)benzamide;
(R)-5-Ethyl-2-fluoro-4-methyl-N-(3-methyl-1-(4-methyl-4-(4-(methylsulfonyl)phenylamino)piperidin-1-yl)-1-oxobutan-2-yl)benzamide;
(R)-5-Ethyl-2-fluoro-N-(3-methyl-1-(4-methyl-4-((4-(methylsulfonyl)phenylamino)methyl)piperidin-1-yl)-1-oxobutan-2-yl)benzamide;
(R)-2-Fluoro-5-isopropyl-N-(3-methyl-1-(4-methyl-4-(4-(methylsulfonyl)phenylamino)piperidin-1-yl)-1-oxobutan-2-yl)benzamide;
(R)-2-Fluoro-N-(3-methyl-1-(4-methyl-4-(4-(methylsulfonyl)benzamido)piperidin-1-yl)-1-oxobutan-2-yl)-5-(trifluoromethoxy)benzamide;
(R)—N-(3-Methyl-1-(4-methyl-4-(4-(methylsulfonyl)benzamido)piperidin-1-yl)-1-oxobutan-2-yl)-3-(trifluoromethoxy)benzamide;
(R)-2-Fluoro-N-(3-methyl-1-(4-methyl-4-((4-(methylsulfonyl)benzyl)amino)piperidin-1-yl)-1-oxobutan-2-yl)-5-(trifluoromethoxy)benzamide;
(R)-2-Fluoro-N-(1-(4-((4-methoxybenzyl)amino)-4-methylpiperidin-1-yl)-3-methyl-1-oxobutan-2-yl)-5-(trifluoromethoxy)benzamide;
(R)—N-(3-Methyl-1-(4-methyl-4-((4-(methylsulfonyl)benzyl)amino)piperidin-1-yl)-1-oxobutan-2-yl)-3-(trifluoromethoxy)benzamide;
(S)—N-(3-Methyl-1-(4-methyl-4-((4-(methylsulfonyl)benzyl)amino)piperidin-1-yl)-1-oxobutan-2-yl)-3-(trifluoromethoxy)benzamide;
(R)—N-(1-(4-((4-Methoxybenzyl)amino)-4-methylpiperidin-1-yl)-3-methyl-1-oxobutan-2-yl)-3-(trifluoromethoxy)benzamide;
or a pharmaceutically acceptable salt thereof.

45. A pharmaceutical composition comprising the compound or salt according to any one of claims 1 to 44, formulated with or without one or more pharmaceutical carriers.

46. A method for the treatment of at least one of cancer, lymphocyte homing, chronic inflammation, neuropathic pain, fibrotic diseases, thrombosis, and cholestatic pruritus mediated at least in part by ATX comprising administering to a subject in need thereof a therapeutically effective amount of a compound or salt according to any one of claims 1 to 44 or a composition of claim 45.

47. A method for the treatment of at least one of cancer, lymphocyte homing, chronic inflammation, neuropathic pain, fibrotic diseases, thrombosis, and cholestatic pruritus comprising administering to a subject in need thereof a therapeutically effective amount of a compound or salt according to any one of claims 1 to 44 or a composition of claim 45 that binds to and inhibits ATX providing a reduction in LPA levels.

48. A method of treating fibrosis, inflammation, cancer, angiogenesis, or pain in a mammal comprising administering a therapeutically effective amount of a compound according to any one of claims 1 to 44 or a composition of claim 45, or a pharmaceutically acceptable salt thereof, to the mammal in need thereof.

49. A method of treating lung fibrosis, asthma, chronic obstructive pulmonary disease (COPD), renal fibrosis, acute kidney injury, chronic kidney disease, liver fibrosis, skin fibrosis, fibrosis of the gut, breast cancer, pancreatic cancer, ovarian cancer, prostate cancer, glioblastoma, bone cancer, colon cancer, bowel cancer, head and neck cancer, melanoma, multiple myeloma, chronic lymphocytic leukemia, B cell lymphoma, T cell lymphoma, cancer pain, tumor metastasis, transplant organ rejection, scleroderma, ocular fibrosis, age related macular degeneration (AMD), diabetic retinopathy, collagen vascular disease, atherosclerosis, Raynaud's phenomenon, rheumatoid arthritis, osteoarthritis or neuropathic pain in a mammal comprising administering a therapeutically effective amount of a compound according to any one of claims 1 to 44 or a composition of claim 45, or a pharmaceutically acceptable salt thereof, to the mammal in need thereof.

50. The method according to any one of claims 46-49, comprising administering to the mammal one or more additional therapeutically active agents selected from: corticosteroids, immunosuppressants, analgesics, anti-cancer agents, anti-inflammatories, non-steroidal anti-inflammatories, dual cyclooxygenase-1 and -2 inhibitors, cyclooxygenase-2 selective inhibitors, TNFα blockers, kinase inhibitors, chemokine receptor antagonists, bronchodilators, leukotriene receptor antagonists, leukotriene formation inhibitors, prostaglandin receptor antagonists, prostaglandin formation inhibitors, monoacylglycerol kinase inhibitors, phospholipase A1 inhibitors, phospholipase A2 inhibitors, lysophospholipase D (lysoPLD) inhibitors, autotaxin inhibitors, and LPA receptor antagonists.
